(19) United States
(12) Patent Application Publication
(10) Pub. No.: US 2024/0416137 A1
(43) Pub. Date: Dec. 19, 2024

(54) PERSONALIZED BIOELECTROMAGNETIC THERAPEUTICS

(71) Applicant: OCTANE INNOVATION INC., Kingston (CA)

(72) Inventors: Timothy J.N. SMITH, Kingston (CA); lan GRANT, Picton (CA); Mark D. JERONIMO, Kingston (CA)

(21) Appl. No.: 18/704,244

(22) PCT Filed: Oct. 7, 2022
(86) PCT No.: PCT/CA2022/051490

§ 371 Date: Apr. 24, 2024

Related U.S. Application Data
(60) Provisional application No. 63/253,850, filed on Oct. 8, 2021.

(51) Int. Cl. A61N 2/02; A61N 2/00; C12Q 1/6851; C12Q 1/6876

(52) U.S. Cl. CPC A61N 2/02; A61N 2/004; A61N 2/008; C12Q 1/6851; C12Q 1/6876; C12Q 2600/158

(57) ABSTRACT

A bioelectromagnetic treatment method for delivering personalized, effective bioelectromagnetic therapy to a microenvironment injury and disease site of a patient undergoing the therapy. The bioelectromagnetic treatment method is self-adaptive and bio-responsive to the microenvironment undergoing treatment and suitable for the treatment of injury and disease.

### FIELD

The application generally relates to methods, devices and systems for providing personalized intelligent self-adaptive bioelectromagnetic therapy.

### BACKGROUND

Bioelectromagnetic therapy has been used as a non-invasive physical therapy to address injury and disease, including cancer, bone non-union fractures and pain. For example, in the case of a bone fracture, studies have shown that the rate of non-union of a fracture may be reduced in people who have used electromagnetic stimulation in their treatment. Contradictory studies, however, show minimal or no benefit of using bioelectromagnetic therapy for bone healing.

One reason for inconsistent clinical efficacy of bioelectromagnetic therapy is due to the subjective nature of determining dosages and regimens of electrical stimulation by the medical practitioner. A clinician may select protocols based on animal studies or based on protocols used for other patients. As there is no rigorous way to determine the ideal physical parameters for the applied bioelectromagnetic therapy, clinicians have generally adopted a therapeutic approach based on a limited sense of historical equivalencies. However, the lack of optimized conditions inevitably leads to variability in clinical outcomes, including treatment failure. This variability has precluded bioelectromagnetic therapy becoming a standard of care for applicable diseases and injuries. In addition to variability, many protocols and systems used for applying bioelectromagnetic therapy are inconvenient and impractical to use leading to poor patient compliance and other undesirable complications.

Knowledge processing has been incorporated into treatment methods in order to try to customize a treatment profile more closely to that of the patient. Generally, these methods require the generation of a profile of the patient that includes demographics, physiological data and characteristics of the condition for which treatment is sought. The patient profile is compared to a patient analytics database that comprises data compiled on a plurality of individuals to determine a recommended therapy, based on matching the patient profile to an individual with the most similar characteristics. While treatment outcome may be improved, this approach is not truly unique to the patient, but rather, most similar to the matched historical individual.

Identifying a less subjective and more personalized treatment approach for patient bioelectromagnetic therapy is projected to lead to more optimal therapeutic outcomes.

The above discussion is not intended as an admission that any of the foregoing is pertinent prior art.

### SUMMARY

In view of the foregoing limitations and shortcomings of presently used bioelectromagnetic treatment methods, as well as other disadvantages not specifically mentioned above, a more precise and effective bioelectromagnetic therapy approach is desired.

There are several variables involved with presently used approaches and regimens for administering therapeutic electrical fields to a target area for treatment. Electrical fields can be applied using direct current via implanted electrodes at the treatment target site, by the generation of transient alternating currents at the treatment target site using capacitively-coupled electrodes, or inductive stimulation using coils to generate an electromagnetic field (EMF) at the treatment target site. With respect to capacitively-coupled or inductively-coupled EMF treatments, a variety of electrode or coil arrangements and designs are possible. Electrodes/coils are positioned in close proximity to the target site of treatment. Problems may arise due to self-inductance and background electrical interference. Depending on the size of the target treatment area, a wider spacing of the active components may be required causing a decrease in the EMF strength. Further, electrodes/coils contoured to a curvature of the treatment area may distort the induced field leading to variable treatment results.

There is also considerable variability with respect to the biology of a patient that may influence the outcome of EMF treatment. For example, variability exists due to the patient's biometrics, genetics, medical history, comorbidities and high-risk lifestyle factors. Variability also exists with respect to the type of injury, trauma, disorder or disease. Injuries may be superficial or within a deeper structure. The tissue and cellular milieu of an injury is unique with the cells having an influence on electrical activation and activation selectivity.

There is also variability in the starting point for adoption of bioelectromagnetic therapy within the progression of the disease or injury repair along with inherent changes in the required biological cascade throughout the treatment process.

Living tissues comprise complex cellular architectures. Cells of the body have built in electromagnetic attributes such that they are exquisitely responsive to electromagnetic stimulation of exactly the right frequency and amplitude. The endogenous bioelectric field and current transmission in a tissue in turn affects: cell membrane capacitance, permeability of the cell membrane, signaling mechanisms of the cell membrane, intracellular mineral concentrations, nutrient flow into the cell and waste disposal. The composition and degree of injury of a tissue will affect the bioelectric field and the flow of current therein. Bioelectromagnetic therapy can trigger or enhance inherent bioelectric events underway within the cells and tissues in response to injury or disease and thus assist in healing and repair.

The cellular architecture of an injury/disease varies upon the length of time since the occurrence of the injury/disease and/or concurrent biological events such as inflammation, and potential re-injury of the same tissue in a subject. This is more pronounced between different subjects for a variety of reasons such as differences in age, gender and presence of any underlying health factors. It is therefore not clinically sound to provide the same treatment regime for different people even with the same type of injury/disease and expect the same therapeutic outcome. Providing the correct and required stimulating signal to promote proper growth and differentiation is an inherently unique feature of the type and location of cells and stage of healing of the patient at that particular time of treatment.

The interplay of the aforementioned variabilities have provided a basis for development of the personalized bioelectrotherapeutic approach disclosed herein.

Personalized bioelectrotherapeutic therapy for treating biological tissues should generate bioelectric stimulation with sufficient intensity, format, duration and temporal sequence to be capable of activating a cascade of cellular signaling processes and extracellular signals, initiate enzyme reactions, membrane transport, cell proliferation and differentiation and other biological processes involved in healing and repair without being so strong that the generated currents create undesirable physiological reactions. The electromagnetic field (EMF) should satisfy the frequency, amplitude, and temporal pattern that native and repair cells innately possess, require and expect for proper growth and differentiation during the process of healing.

It has not previously been realized that parameters external to the injury or disease microenvironment related to the generation and transmission of a therapeutic electromagnetic field are tied to, and thus intimately related to, the ability of providing therapeutic bioelectromagnetic stimulation to satisfy requirements of the injury or disease microenvironment targeted for treatment in a given patient. Nor was it previously realized that even subtle differences in one or more of the variabilities involved can render a bioelectromagnetic therapy ineffective between different patients even for the same type of anatomical injury/disease.

Accordingly, the invention provides bioelectromagnetic therapy methods, devices and systems that comprise multiple points of personalization throughout a bioelectromagnetic treatment protocol thus compensating for variabilities associated with the biological challenge, the patient profile, the EMF transmission pathway, and device deployment specifics in order to stimulate a desired outcome.

The complex relationship between the variable biological parameters of the patient and the microenvironment of the treatment area and the variable parameters of the electromagnetic treatment modality prescribed for the patient are computationally defined in order to provide an effective personalized electromagnetic treatment protocol specific for the patient.

Described herein are bioelectromagnetic therapy methods, devices and systems that advantageously incorporate specialized and intelligent physics-based computational engines to provide a more robust, comprehensive, and effective approach to deliver personalized bioelectromagnetic therapy. The proprietary computational engines are configured to employ artificial intelligence, machine learning, computational and mathematical analysis—to organized collections of data representing multidimensional parameters relating to: (a) biology of the patient, and the biology of the patient's microenvironment targeted for treatment, inclusive of clinical metadata pertaining to the biology of similar patient and target microenvironment; and (b) the electromagnetic treatment modality prescribed for the patient that emit electromagnetic fields to influence the target microenvironment, this includes parameters affecting electromagnetic field deployment and electromagnetic field transmission efficiency, in order to generate a personalized treatment for a patient.

The proprietary computational engines are configured to enable determination of an ideal electromagnetic field requirement directed at the microenvironment targeted for treatment of a subject and further determine a personalized electromagnetic field treatment protocol for the subject inclusive of a precise means to deliver the determined ideal electromagnetic field requirement during the personalized treatment protocol.

The personalization of a treatment for a patient as herein described is not based on a historical database comparison and not a general patient-matching treatment model. To the contrary, the personalized treatment protocol as herein described is to be an exact match for the requirements of the patient microenvironment being treated with a prescribed electromagnetic treatment modality in order to more exactly influence healing at cellular and molecular levels and/or act on mediators of inflammation, and/or act on biological factors to provide faster healing of injury, increased quality of healing, reduction in disease progression and/or pain management.

The limits of the bioelectromagnetic field stimulation applied during a personalized treatment protocol is not predetermined, but instead established specifically to match the microenvironment of a target in a patient and thereafter self-adaptively adjusts responsive to changes occurring at the cellular level of the target as a result of the applied electric signal over the course of the treatment protocol.

Embodiments of the invention disclosed herein include in aspects, a Microenvironment Computational Engine (MiCE) configured to use organized indexed collections of data representing multidimensional parameters of the patient and the biology of the patient's microenvironment targeted for treatment (patient-centric data), and clinical metadata pertaining to the biology of similar patients and target microenvironments for calculating a patient-specific and theoretically ideal Personalized Microenvironment Stimulation Target (PMST). The PMST is the calculated ideal electromagnetic field stimulation required for the microenvironment targeted for treatment.

Embodiments of the invention disclosed herein include in aspects a Macrotranslation Computational Engine (MaCE) configured to use organized indexed collections of data representing multidimensional parameters of the prescribed electromagnetic field modality (EMF modality-centric data) to calculate the precise means to deliver the ideal electromagnetic field requirement (i.e., the PMST). The MaCE outputs the initial settings characterizing the EMF source, referred to as the Personalized Treatment Protocol (PTP). EMF modality-centric data represents: (a) transmission pathway data representing the signal pathway separating the microenvironment and EMF source that is influenced by material properties and physical dimensions and movement of tissues and materials along the signal pathway; and (b) deployment specifics data representing the modality of electromagnetic field generation and physical construction of EMF signal generator device as configured to the patient and the injury or disease.

Embodiments of the invention disclosed herein may further comprise optimization extension.

In aspects the optimization extension comprises a Feedback Computational Engine (FCE) configured for dynamic sensing, calculation and adaptive correction of the MaCE.

In aspects the optimization extension comprises a Learning Computational Engine (LCE) configured for dynamic clinical sensing, calculation and adaptive correction of the MiCE.

In aspects the optimization extension comprises both an FCE and an LCE.

Herein described are computer implemented methods, devices and systems comprising MiCE and MaCE and optionally one or both of FCE and LCE that overcome at least one shortcoming of previously described electromagnetic methods and for generation and application of personalized bioelectrical signals optimized to provide an exact stimulus required and unique to the injury or disease microenvironment of the patient resulting in the desired biological response.

A desired biological response for injury or disease may comprise alterations in biological processes at the microenvironment involved in for example but not limited to: stabilizing, reversing and/or improving state of injury or disease; improving/restoring function of injury or tissue/organ affected by disease; decrease spread/growth of disease; stabilize injury or disease; manage/decrease pain associated with injury or disease.

In aspects, the personalized electromagnetic field signals as described herein more efficiently match frequency components to a relevant cellular/molecular process of the injury or disease microenvironment of the patient for which it was calculated.

In aspects, the personalized electromagnetic field signals as described herein correspond more directly to signals of the injury or disease microenvironment of the patient for which it was calculated resulting in accelerated healing.

In aspects the personalized electromagnetic field signals as described herein more precisely target biochemical and biophysical pathways of cells and associated structures in the injury or disease microenvironment encouraging cellular growth, tissue growth, repair, and maintenance.

In aspects, the application of the personalized electromagnetic field signals as described herein may stimulate action of growth factors and other cytokines of the targeted microenvironment.

In aspects, the application of personalized electromagnetic field signals as described herein may modify the genetic regulation of cells within the targeted microenvironment.

In aspects, the personalized electromagnetic field signals as described herein may have decreased effects on off target cells/tissues.

In aspects the personalized electromagnetic field is for application for a time effective for the injury to substantially heal.

In aspects the personalized electromagnetic field is applied for a time effective to reverse, stabilize and/or cure the disease.

In aspects, the personalized electromagnetic field is a pulsed electromagnetic field (PEMF).

In aspects, the personalized electromagnetic field is a capacitively-coupled electric field.

According to an aspect of the invention is device for providing an electromagnetic field (EMF) to an injury or disease in a patient, the device comprising:
an EMF signal generator configured to generate a personalized electromagnetic field signal for a microenvironment target to match requirements unique to the injury or disease of the patient; and
at least one EMF source in operable communication with the signal generator to deliver/apply the personalized electromagnetic field signal to the injury or disease.

In aspects, the device is configured to deliver a personalized electrical stimulation field to preferentially stimulate (up-regulate, down-regulate, or a combination of both) the biochemical cellular and sub-cellular molecular responses to trigger the activation of known mammalian genes responsible for the regeneration, restoration, repair, maintenance, or any combination of cartilage, bone, or both.

In aspects, the device is configured to deliver a personalized electrical stimulation field to preferentially stimulate (up-regulate, down-regulate, or a combination of both) the biochemical cellular and sub-cellular molecular responses unique to the patient microenvironment to trigger the activation of known mammalian genes responsible for pain regulation, pain relief, and/or pain reduction.

In aspects, the device is configured to deliver a personalized electrical stimulation field to preferentially stimulate (up-regulate, down-regulate, or a combination of both) the biochemical cellular and sub-cellular molecular responses unique to the patient microenvironment to trigger the activation of known mammalian genes responsible for slowing down or reversing cancer growth.

In aspects, the device is configured to deliver a personalized electrical stimulation field to preferentially stimulate (up-regulate, down-regulate, or a combination of both) the biochemical cellular and sub-cellular molecular responses to trigger the activation of known mammalian genes unique to the patient microenvironment responsible for general feeling of well being, or reduction in one or more symptoms of anxiety, or reduction in one or more symptoms of stress.

In aspects, the device is configured to deliver a personalized electrical stimulation field to preferentially stimulate (up-regulate, down-regulate, or a combination of both) the biochemical cellular and sub-cellular molecular responses unique to the patient microenvironment to trigger the activation of known mammalian genes responsible for slowing down or reversing or managing a neurological disorder.

In aspects, the EMF signal applicator is configured for inductive coupling with the EMF source(s), e.g. a coil(s), or capacitive coupling using electrode(s) for electrochemical contact with surface of the treatment target.

In aspects the electromagnetic (EMF) signal generator comprises the engine means, processor(s) and memory for generating and delivering the personalized programmed treatment protocol to satisfy the requirements of the personalized microenvironment stimulation target at the injury or disease site of a patient.

In aspects the EMF signal generator may further comprise a display and touch pad or input keys allowing for patient interaction or navigation within the display. In aspects, the EMF may form a kit or part of a kit with instructions. In aspects, the EMF signal generator may be in operable communication with one or more remote operational networks.

In aspects, the device is configured as a wearable device comprising an anatomical wrap, anatomical support (e.g. brace), apparel (e.g. t-shirt, sweat shirt), chest support (e.g. bra), hat/cap/helmet, foot ware (e.g. insoles for sneakers, boots), fashion accessory (e.g. bracelet), dressing, bandage, compression bandage and compression dressing.

In aspects, the device and/or components thereof is configured to be re-useable.

In aspects, the device is configured to be re-programmable.

In aspects, the device and/or components thereof are configured to be disposable, recyclable and/or replaceable.

In aspects, the device and/or components thereof are configured to be implantable in a patient.

In aspects the device and/or components thereof is configured to be integrated into a mattress, mattress pad, linen (sheets, pillowcases), furniture (e.g. bed, chair, sofa), exercise equipment, or support device (e.g. wheelchair) onto which the subject may sit, recline, etc.

According to an aspect of the invention is a method personalized for treatment of an injury or disease in a patient, the method comprising:
applying an electromagnetic field to the injury or disease, wherein said electromagnetic field provides bioelectrical signals of an exact stimulus required and unique to the injury or disease of the patient for healing.

In aspects, the personalized electromagnetic field is generated incorporating biological data parameters of the injury or disease of the patient.

In aspects, the personalized electromagnetic field is generated incorporating biological data parameters of the patient relevant to the injury or disease.

In aspects, the personalized electromagnetic field is generated incorporating clinical metadata relevant to the injury or disease.

In aspects, the personalized electromagnetic field is generated incorporating clinical metadata relevant to the injury or disease.

According to an aspect of the invention is an electromagnetic field (EMF) treatment system personalized for a patient comprising:
a Microenvironment Computational Engine (MiCE) configured for calculating a theoretically ideal Personalized Microenvironment Stimulation Target (PMST) for delivery of electric stimulation to a microenvironment of a patient; and
a Macrotranslation Computational Engine (MaCE) configured for calculating a precise means for delivering the ideal personalized microenvironment stimulation target electromagnetic field to the microenvironment of the patient as a personalized treatment protocol.

In aspects, the Microenvironment Computational Engine (MiCE) comprises protocols to integrate and process parameter data based on the patient, the biology of the patient's microenvironment, and clinical metadata pertaining to the biology of similar target microenvironment.

In aspects, the Macrotranslation Computational Engine (MaCE) comprises protocols to integrate and process parameter data based on the EMF treatment modality and patient factors external to the microenvironment to deliver the ideal personalized electromagnetic field to the microenvironment of the patient.

According to an aspect of the invention is a wearable electromagnetic field (EMF) therapy system for treating an injury or disease in a patient, the system comprising:
an EMF signal device;
a microcontroller;
one or more flexible coil wire EMF sources coupled to the EMF signal device; and
a material configured to secure the EMF sources over the injury or disease area;
wherein the microcontroller is configured to generate a personalized treatment protocol that delivers an ideal personalized EMF stimulation unique to the microenvironment of the injury or disease.

According to an aspect of the invention is a computer implemented electromagnetic field (EMF) treatment system comprising:
an electromagnetic field (EMF) signal generator comprising a memory or chip storing:
a microenvironment computational engine for calculating a personalized microenvironment stimulation target; and
a macrotranslation computational engine for generation of a personalized treatment protocol based on the ideal personalized electromagnetic field; and
one or more EMF sources coupled to the EMF signal generator for applying the ideal personalized electromagnetic field to the patient.

According to a further aspect is a bioelectromagnetic therapy system comprising:
a microenvironment computational engine configured to calculate a theoretically ideal personalized microenvironment target specific to a treatment target of a patient;
a macrotranslation computational engine configured to generate a personalized treatment protocol based on the theoretically ideal personalized microenvironment target; and
a signal generator device configured to emit the personalized treatment protocol.

The system, device and methods described herein are suitable for personalization of treatments for any variety of clinical indications including but not limited to treatment of injury, disease, pain management, physical effects of stress and/or anxiety and for whole body systemic benefit. The system, device and method of the invention may also in aspects be suitable as a preventative strategy for a recurring condition.

The personalized, intelligent self-adaptive bioelectromagnetic therapy of the invention is well tolerated by a patient, need not be invasive or unpleasant, does not cause pain and thus is beneficial for increasing patient treatment compliance. The method does not require manual adjustment of power, pulse rate duration, pulse width duration, or treatment time by the treatment provider. A bioelectromagnetic therapy device programmed and configured to deliver the personalized, intelligent self-adaptive bioelectromagnetic therapy as herein described benefits from convenience of use, versatility and ability for treatment of single or multiple treatment targets on the same patient or alternatively for full body treatment of a patient.

This comprehensive personalized approach improves the efficacy of the bioelectromagnetic treatment resulting in desired and more enduring therapeutic outcomes. The method is also advantageous as it calculates the EMF treatment protocol specific to a treatment target area of a patient and thus substantially avoids possible compromising effects to tissues in close proximity.

From the foregoing, it will be appreciated by those skilled in the art that the present invention provides a particularly effective method, device and system for overcoming many of the limitations associated with the treatment of patients using conventional electromagnetic energy. It will also be readily appreciated by one with ordinary skill in the art that the invention is suitable for treatment of humans and also has veterinary applications.

These and other features, embodiments, and advantages of the present disclosure are mentioned not to limit or define the disclosure, but to provide examples to aid in the understanding thereof when read with the following Description and with reference to the accompanying drawings.

### BRIEF DESCRIPTION OF FIGURES

For a more complete understanding of the present disclosure, reference is made to the following description taken in conjunction with the accompanying drawings.

FIG. 1: Operational components of a bioelectromagnetic therapeutic device according to embodiment of the present disclosure;

FIG. 2: Abstraction of interconnected factors contributing to the effective generation and delivery of an electromagnetic field capable of producing a desired biological response at the microenvironment;

FIG. 3: Operational flowchart showing the core elements of a personalized bioelectricity treatment protocol of the invention;

FIG. 4: Operational flowchart for a personalized bioelectricity treatment protocol for bone, illustrating a tibial non-union fracture according to an embodiment of the present disclosure;

FIGS. 5(A)-(C): (A) Simplified implementation of an electromagnetic source device depicting an arrangement of the components in conjunction with a tibial non union fracture; (B) illustrates the configuration of parallel coils and a shaped coil; and (C) is an abstraction of generated electromagnetic fields surrounding the microenvironment of the tibial non union fracture.

FIG. 6: Operational flowchart for a personalized bioelectricity treatment protocol for treatment of cancer, illustrating a glioma (cancer) according to an embodiment of the present disclosure;

FIGS. 7(A)-7(B): (A) Simplified implementation of an electromagnetic source device depicting an arrangement of the components in conjunction with a glioma; and (B) an abstraction of generated electromagnetic fields surrounding the microenvironment of the glioma;

FIG. 8: Operational flowchart of a personalized bioelectricity treatment protocol for treatment of chronic pain, illustrating chronic knee pain associated with knee osteoarthritis according to an embodiment of the present disclosure;

FIGS. 9(A)-9(B): (A) Simplified implementation of an electromagnetic source device depicting an arrangement of the components in conjunction with an osteoarthritic knee; (B) Abstraction of generated electromagnetic fields surrounding the microenvironment of the osteoarthritic knee;

FIG. 10: Arrangement showing cell culture plates exposed to a spatially uniform, time-varying magnetic field using Helmholtz coils;

FIG. 11: Sample PCR array heatmap showing increased and decreased gene expression of ‘test’ sample versus a calibrator (‘control’ cells/donor). In this example, unstimulated PC-1 gene expression levels are plotted relative to unstimulated LZ-1 levels. Each grid position is annotated with the fold change (FC), which was plotted to log-base 2 such that zero corresponds to no difference in expression levels between the test and calibrator specimens. Cross-hatched boxes denote genes that had negligible expression in both the test and control samples;

FIG. 12: Array of gene expression heatmaps illustrating the differences between baseline gene expression (cultured without EMF stimulation) for each donor relative to the others. Heatmaps should be identified as Row (Test) vs Column (Control/Calibrator) and PC-1 vs LZ-1 is the exact inverse of LZ-1 vs PC-1. The single colour-bar applies to all heatmaps;

FIG. 13(A)-(B): Heatmaps showing the inter-donor variability when cells are cultured in the presence of alternating magnetic fields: (A) 75 Hz, 4 mT pulse waveform; and (B) 50 Hz, 1 mT sine wave. Gene expression varies significantly between donors in the pulsed field. Each heatmap is normalized and represents exposed vs unexposed control of the same donor. The colour-bar applies to all ten heatmaps;

FIG. 14: Graph showing the Fold change of the expression of ten osteogenic genes for each donor (compared to their own control) that were selected from the Experiment 1 PCR arrays. Values less than 1 represent a down-regulation and greater than 1 is an up-regulation of that gene;

FIG. 15: Heatmaps of cells Donor PC-1 were exposed to six different forms of stimulation. Pulsed magnetic fields (top row) were varied in daily exposure time (10 min/day to continuous), whilst sine waves (bottom row) were evaluated at three different frequencies (15-250 Hz). The cross-hatching denotes negligible gene expression from the test and control samples;

FIG. 16: Graph showing expression of osteogenic genes affected by pulsed and sinusoidal magnetic field exposures;

FIG. 17: Graph showing change of expression of chondrogenic genes affected by pulsed and sinusoidal magnetic field exposures;

FIG. 18(A)-(B): Graph showing cell counts following culture of MDA-MB-231 cells with EMF exposure (A) there are statistically significant differences between the unexposed control and multiple experiments. The stimulation profiles used in Experiments 3 and 4 have inhibited breast cancer cell growth. The exposures were repeated with chondrocytes (B) demonstrating that there is no statistically significant effect on the growth of non-cancerous cells;

FIG. 19(A)-(B): Heatmaps corresponding to the gene expression changes, relative to non-stimulated control, created by the stimulation profiles in Experiments 3 (A) and 4 (B). Genes belonging to this Cancer Pathway Finder PCR array are involved in pathways including apoptosis, cellular senescence, and angiogenesis. Cross-hatching denotes genes with negligible expression;

FIG. 20(A)-(B): Graphs showing normalized cell counts for cultures performed with and without cisplatin and/or EMF exposure. Cisplatin vehicle has no significant effect on proliferation, but a high concentration of the drug is inhibitory (A). Relative to the unstimulated vehicle, the addition of low concentrations of cisplatin and/or EMF exposure significantly lowered cell counts (B). The additional benefit provided by Experiments 3 and 4, at either concentration, is statistically significant compared to the chemotherapy drug alone;

FIG. 21: Representative images (50× magnification) of donor NHA-2 astrocytes prior to termination. Images A and B are images of non-reactive and reactive control cell groups, respectively;

FIG. 22: Graphs showing quantitative PCR results for select genes used to verify the reactive state of the astrocytes. As confirmed here, the reactive (or pain) state exhibits low levels of GFAP, TGFB, STAT3 and SOX9, and elevated expression of IL6, TNFα, IL8, IL-1β and C3, compared to non-reactive control levels. The vertical axis shows the mRNA level of the gene of interest (GOI) relative to house-keeping gene GAPDH;

FIG. 23: Heatmap of the pain PCR array revealing the fold change differences between the baseline expression levels of NHA2 cells in a reactive state compared to a non-reactive state (calibrator). Red and blue boxes represent up-regulation and down-regulation, respectively, of the gene expression relative to non-reactive cells. Gray boxes denote genes with negligible expression in both the test and calibrator conditions.

FIG. 24: Array of gene expression heatmaps illustrating donor-to-donor baseline gene expression variability. Comparisons within the table are identified as Row (Test) vs Column (Calibrator) and maps on one side of the diagonal are the exact inverse of the opposite side. The colour-bar applies to all heatmaps. Cross-hatching denotes genes that had negligible expression;

FIG. 25: Heatmaps demonstrate inter-donor differences when cells from three donors (top, NHA1; centre, NHA2; bottom, NHA3) are subjected to the same EMF stimulation (Experiment 1). Exposure strongly up-regulated many genes for NHA1 relative to its reactive control, whilst the majority of the genes are down-regulated in NHA3;

FIG. 26: Graph showing gene expression levels of NHA1, NHA2 and NHA3 cells when exposed to an extremely-low frequency and low intensity magnetic field for 10 minutes per day (Experiment 1). Select inflammatory genes are shown. Genes that are up-regulated have positive values and vice versa for down-regulated gene expression;

FIG. 27: Heatmaps show changes to gene expression for 84 unique genes responsive to stimulations of the NHA2 cell group. Experiment 1 (left), Experiment 2 (middle) and Experiment 3 (right). Quantities represent log 2 (fold change), where the fold change is relative to the NHA2 reactive controls. Cross-hatched boxes are genes that had negligible expression in both the test and control samples; and

FIG. 28: Graph demonstrating intra-donor variability when cells from the same donor group (NHA2) tare exposed to three different EMF stimulation profiles (labeled Experiment 1-3). Gene expression of the exposed cells is plotted relative to reactive controls. The same genes are strongly up-regulated by the transition from non-reactive to reactive astrocytes.

### DESCRIPTION

Reference will now be made in detail to embodiments, examples of which are illustrated in the accompanying drawings. In the following detailed description, numerous specific details are set forth in order to provide a sufficient understanding of the subject matter presented herein. But it will be apparent to one of ordinary skill in the art that the subject matter may be practiced without these specific details. Moreover, the particular embodiments described herein are provided by way of example and should not be used to limit the scope of the invention to these particular embodiments. In other instances, well-known data structures, timing protocols, software operations, procedures, and components have not been described in detail so as not to unnecessarily obscure aspects of the embodiments of the invention.

As used herein, the terms “invention” or “present invention” are non-limiting terms and not intended to refer to any single aspect of the particular invention but encompass all possible aspects as described in the specification and the claims.

All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in the art to which the subject matter herein belongs. It will be further understood that terms, such as those defined in commonly used dictionaries, should be interpreted as having a meaning that is consistent with their meaning in the context of the relevant art and the present disclosure, and will not be interpreted in an idealized or overly formal sense unless expressly so defined herein.

Reference to “one embodiment,” “an embodiment,” “a preferred embodiment” or any other phrase mentioning the word “embodiment” means that a particular feature, structure, or characteristic described in connection with the embodiment is included in at least one embodiment of the-disclosure and also means that any particular feature, structure, or characteristic described in connection with one embodiment can be included in any embodiment or can be omitted or excluded from any embodiment. The appearances of the phrase “in one embodiment” in various places in the specification are not necessarily all referring to the same embodiment, nor are separate or alternative embodiments mutually exclusive of other embodiments. Moreover, various features are described which may be exhibited by some embodiments and not by others and may be omitted from any embodiment. Furthermore, any particular feature, structure, or characteristic described herein may be optional. Similarly, various requirements are described which may be requirements for some embodiments but not other embodiments. Where appropriate any of the features discussed herein in relation to one aspect or embodiment of the invention may be applied to another aspect or embodiment of the invention. Similarly, where appropriate any of the features discussed herein in relation to one aspect or embodiment of the invention may be optional with respect to and/or omitted from that aspect or embodiment of the invention or any other aspect or embodiment of the invention discussed or disclosed herein.

It will be understood that any component defined herein as being included in any described embodiment may be explicitly excluded from the claimed invention by way of proviso or negative limitation.

As used herein, the articles “a” and “an” preceding an element or component are intended to be non-restrictive regarding the number of instances (i.e. occurrences) of the element or component. Therefore, “a” or “an” should be read to include one or at least one, and the singular word form of the element or component also includes the plural unless the number is obviously meant to be singular.

It will be further understood that the terms “comprises” and/or “comprising,” or “includes”, “including” and/or “having” and their inflections and conjugates denote when used in this specification, specify the presence of stated features, regions, integers, steps, operations, elements, and/or components, but do not preclude the presence or addition of one or more other features, regions, integers, steps, operations, elements, components, and/or groups thereof. Words using the singular or plural number also include the plural or singular number respectively. Additionally, the words “herein,” “hereunder,” “above,” “below,” and words of similar import refer to this application as a whole and not to any particular portions of this application.

As used herein, the term “about” refers to variation in the numerical quantity. In one aspect, the term “about” means within 10% of the reported numerical value. In another aspect, the term “about” means within 5% of the reported numerical value. Yet, in another aspect, the term “about” means within 10, 9, 8, 7, 6, 5, 4, 3, 2, or 1% of the reported numerical value.

“About,” is equivalent to “approximately,” or “substantially” as used herein and inclusive of the stated value and means within an acceptable range of deviation for the particular value as determined by one of ordinary skill in the art, considering the measurement in question and the error associated with measurement of the particular quantity (i.e., the limitations of the measurement system). For example, “about,” “approximately,” or “substantially” can mean within one or more standard deviations, or within +30%, 20%, 10%, 5% of the stated value.

Should a range of values be recited, it is merely for convenience or brevity and includes all the possible sub-ranges as well as individual numerical values within and about the boundary of that range. Any numeric value, unless otherwise specified, includes also practical close values and integral values do not exclude fractional values. Ranges given herein also include the end of the ranges.

As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. Accordingly, specific values recited for radicals, substituents, and ranges, are for illustration only; they do not exclude other defined values or other values within defined ranges for radicals and substituents.

As used herein the term ‘may’ denotes an option or an effect which is either or not included and/or used and/or implemented and/or occurs, yet the option constitutes at least a part of some embodiments of the invention or consequence thereof, without limiting the scope of the invention.

The phrase “and/or,” as used herein in the specification and in the claims, should be understood to mean “either or both” of the elements so conjoined, e.g., elements that are conjunctively present in some cases and disjunctively present in other cases. Other elements may optionally be present other than the elements specifically identified by the “and/or” clause, whether related or unrelated to those elements specifically identified unless clearly indicated to the contrary. When the word “or” is used in reference to a list of two or more s, that word covers all of the following interpretations of the word: any of the s in the list, all of the s in the list and any combination of the s in the list.

As used herein, expressions such as “at least one of,” when preceding a list of elements, modify the entire list of elements and do not modify the individual elements of the list.

“Combination or combining” for the purposes of this invention means any method of putting two or more materials together. Such methods include, but are not limited to, mixing, blending, commingling, concocting, homogenizing, incorporating, intermingling, fusing, joining, shuffling, stirring, coalescing, integrating, confounding, joining, uniting, or the like.

The terms “patient,” “subject,” “individual,” and the like are used interchangeably herein.

In certain non-limiting aspects, the patient, subject or individual is a mammal and includes humans.

As used herein “disease” refers to any abnormal condition that negatively affects the structure or function of all or part of a subject, and that is not due to injury. Diseases are medical conditions associated with specific signs and symptoms such as for example pain and dysfunction and includes for example disorders, syndromes, conditions and mental abnormal behaviors.

As used herein “injury” is damage to the body of a human. Injury can include “trauma” as any injury to human tissues and organs caused by an external force from minor (e.g. cuts and bruises) to critical (e.g. major brain or spinal injuries) and may be categorised as blunt or penetrating.

As used herein “bioelectrical signals” are electrical signals that can be measured from biological beings, for example, humans and include endogenous bioelectrical signals that are produced in cells by the cumulative action of ion channels, pumps, and transporters and transduced into second-messenger responses, and alter aspects of cell behavior. In aspects, low amplitude and low frequency electrical signals.

As used herein “bioelectricity” refers to endogenous electrical potentials and currents occurring within or produced by living cells and tissues.

As used herein “electromagnetic field” (EMF) is a form of waves with both electric and magnetic components, the waves characterised by energy, frequency and wavelength. The EMF is the stimulating signal for providing therapy.

As used herein “pulsed electromagnetic field (PEMF)” refers to a time varying (pulsed) electromagnetic field with frequency and intensity.

“Bioelectromagnetic Therapy” refers to treatment of a subject using electromagnetic fields.

“Electromagnetic Field (EMF) Source” refers to the device/apparatus comprising components that generate an electromagnetic field.

“Stimulate” refers to generating a desired response at the cellular level of the biological microenvironment of the treatment target with the application of the personalized EMF treatment protocol. The desired response may change during the course of treatment, for example, an initial increase in cellular growth and differentiation at the biological microenvironment may at first occur followed by either a maintenance period or a period of decrease in the rate of cellular growth and differentiation and/or activation of further cellular events. This is specific to the particular healing process of the biological microenvironment of the particular injury, trauma, disease or condition of the patient.

As used herein “Personalized” refers to treatment specifically generated and customized for an individual patient, and more specifically to the biological microenvironment of the treatment target of the patient.

“Treatment target” refers to the general anatomical area or tissue receiving bioelectromagnetic therapy and is not limited. Any part of the body can be injured or afflicted with disease. The treatment target can also include the “whole body”.

“Microenvironment” refers to the intricate three-dimensional, dynamic network of tissue architecture of the ‘treatment target’ in terms of its composition of cells, extracellular matrix (ECM) components, soluble factors, and physical forces (e.g., fluid flow and mechanical stress). As a result of a particular type of injury or disease the biological microenvironment faces several physiological and/or biochemical challenges in order to begin the complex process of healing and/or complex tissue growth, such as for example but not limited to management of exudate, bacterial control, maceration of cells/tissues, and dead cells/tissues.

As used herein “Personalized Intelligent Self-Adaptive Bioelectromagnetic Therapy” refers to a novel patient-specific and optimized treatment designed specifically for a single patient using computational engines configured to receive specific structured data that generally relates to (a) the microenvironment, this being data regarding the patient and the microenvironment of the treatment target, (b) macrotranslation factors this being data regarding the EMF treatment deployment and logistics of its use relating to the microenvironment (a) Taken together, this allows calculation of a patient-specific and theoretically ideal calculated electromagnetic stimulation requirement for the desired cellular response within the microenvironment for the specific patient.

The structured data relating to the patient and the microenvironment of the treatment target is defined as:
1. “Biological Challenge” refers to an organized collection of data representing and characterizing the injury or disease inclusive but not limited to: type of injury or disease, the severity of the injury or disease, present state of the disease, success or failure of prior treatments. This includes the physiological and/or biochemical challenges described above. The data categories listed herein are not meant to be limiting.
2. “Patient Profile” refers to an organized collection of data representing comprehensive patient-specific factors that affect the microenvironment such as: demographic information; height, weight, body mass index (BMI); past and current medical conditions such as but not limited to diabetes, allergies, vitamin deficiency, blood conditions, heart conditions, vascular conditions; genetic data which can be full/partial genomic sequencing of the subject, sequencing and identification of specific markers (e.g. differential gene expression); surgical procedures; and social history such as but not limited to smoking, alcohol, cannabis, caffeine and exercise. The data categories listed herein are not meant to be limiting. The combination of biological challenge data and patient profile data comprises the patient-centric data.

Table 1 lists non-exhaustive, non-limiting examples of data that is collected for a patient to undergo personalized bioelectromagnetic therapy,

TABLE ONE
General demographics [symbol] Age, Gender, Ethnicity, Height, Weight, BMI
Pregnant/prior pregnancy/menopause
Clinical Assessment:
Medications previous medications, duration of use; current medications
Antibiotic use
Other usage alcohol, tobacco, illicit drugs
Prior conditions/disease/treatment cardiovascular, endocrine, renal, neurological,
cancer, peripheral vascular disease, autoimmune, infectious disease, bone disease
Bio-clinical assessment
General hematological parameters glucose, oxygen, hemoglobin/hematocrit,
cholesterol, Hemoglobin A1C %, lactate, lactate dehydrogenase, calcium, magnesium,
phosphorus, total protein, white blood cells, platelets, white blood cell differential,
polymorphonuclear cells, STABS, lymphocytes, monocytes, cosinophils, basophils
Blood Chemistry Albumin, Total Protein, Creatinine, Blood Urinary, Nitrogen, Glucose,
Alkaline Phosphatase, SGPT, Calcium Phosphatase
Nutritional Status defi [symbol] ness/markers.
General History of the Injury/disease - prior injury/type/location/size
Previous treatment: yes/no
Yes: treatment duration/type
Response to treatment: healing characteristics/time
Current condition/disease for EMF treatment location/recurrence/treatment
Characterization type/location size:
bio-marker (gene expression)assessment baseline, time points during treatment
selected based on cell(s)/tissue(s) involved in injury/disease.
for prognosis, therapeutic response assessment
Imaging, ultrasound, CT scan, radiography, CAT scan, PET, MRI
Time before seeking treatment.
Other treatments first administered.
EMF Device prescribed by treatment provider device configuration
EMF Treatment Plan Duration
EMF Treatment Plan Follow Up Time points, clinical assessment of healing response.
Presence/Characterization of clinical complications e.g infection/further injury
Timepoint/Resolution
Treatment Compliance/Non-compliance [symbol]
indicates data missing or illegible when filed

3. “Clinical Metadata” refers to an organized collection of data representing published data including but not limited to physiological and biochemical data relating to the injury or disease, outcomes from patients that previously received bioelectromagnetic therapy, and proprietary experimental interim and final data obtained from independent in vitro studies applicable to the type of injury or disease. The data categories listed herein are not meant to be limiting.

“Personalized Microenvironment Stimulation Target” (PMST) refers to a patient-specific and theoretically ideal calculated electromagnetic stimulation requirement for the desired cellular response within the microenvironment for the specific patient. It may define a specific induced current or magnetic field density, as a non-limiting example, within the biological microenvironment.

“Microenvironment Computational Engine” (MiCE) refers to a physics-based engine for calculating (e.g. generates, determines) the Personalized Microenvironment Stimulation Target (PMST) for the patient based on the biological challenge data, the patient profile data and the clinical metadata which is inclusive of proprietary experimental data.

The structured data relating to the macrotranslational factors, data regarding the EMF treatment deployment and logistics of its use relating to the microenvironment (a) is defined as:
1. “Transmission Pathway” refers to an organized collection of data representing patient characteristics that influence the transmission (and induction) of electromagnetic fields through (and in) the media that separates the microenvironment from the EMF source. This includes electrical and mechanical properties of cells and tissues with respect to the treatment target microenvironment, 3-dimensional shapes of anatomical structures, movement of anatomical structures, physical distances within each media, external components (e.g., clothing or air gap), etc. The data categories listed herein are not meant to be limiting.
2. “Deployment Specifics” refers to an organized collection of data representing parameters of the chosen EMF delivery mode and the physical location and construction of the EMF source. For example, treatment could be delivered via electrodes or coils whose number, shape, size, orientation, and positioning selectively tailored to the application. The data categories listed herein are not meant to be limiting. This is also inclusive of the movement of the EMF delivery mode and possible deformation during treatment. The combination of transmission pathway data and deployment specifics data comprises the EMF modality-centric data.

“Macrotranslation Computational Engine” (MaCE) refers to a physics-based engine configured use the PMST and the macrotranslational parameters to calculate/determine the initial output required from the EMF source, i.e., the initial EMF settings that, subject to translational effects along the transmission pathway, meets the required specifications to generate the ‘personalized microenvironment stimulation target’. The output of this engine is the ‘Personalized Treatment Protocol’ (PTP).

“Personalized Treatment Protocol” (PTP) refers to the combination of EMF signal parameters (frequency, intensity, waveform, driving voltage, current delivered to coils/electrodes) that are required to meet the ideal PMST. Additionally, this protocol describes the treatment length, daily exposure times and any temporal variations to the personalized EMF target signal over the course of treatment.

“Feedback Computational Engine” refers to a feedback engine configured for correcting differences, at the biological microenvironment, between the ideal PMST and the actual signal, based on input as measured by one or more EMF sensors at or near the microenvironment during treatment, as a result of inaccuracies in the MaCE or distortion and/or attenuation of the PMST due to changes/perturbations that may occur during the treatment protocol. Corrections are sent as feedback data to modify the MaCE input parameters that are used to calculate the personalized treatment protocol.

“Learning Computational Engine” refers to an optimization engine configured as a self-contained feedback loop, using clinical follow-up data and clinical sensor data (sensing in vivo biological parameters), for optimizing the ‘personalized treatment protocol’ and adaptively correcting any inaccuracies in the ‘microenvironment computational engine’. The learning computational engine can incrementally adjust parameters (including frequency, intensity, waveform, etc.) of the ‘personalized treatment protocol’, monitor and map out their effects, and then select the optimal settings to continue treatment. To improve resolution, the interim and final results of treatment and associated patient information are incorporated into the input parameters of the physics-based computational engines.

Conventional pre-defined or manufacturer-suggested non-specific EMF treatment regimes and frequencies, at any scale, completely lack any form of true clinical personalization that would have the potential to account for not only the application (e.g., disease or injury) but also the patient profile, and hardware specifics for precise EMF deployment and optimization of the treatment protocol over time.

The diverse cellular responses to electromagnetic fields between different patients highlight the therapeutic limitations of predetermined electromagnetic fields proposed as universal patient treatments. The basis of the invention disclosed herein is that the ideal EMF exposure for a specific patient can and should be determined specific to (a) microenvironmental factors included in the biological challenge and patient profile; and (b) macrotranslational factors that describe the transmission pathway and deployment specifics of device implementation. Further, during treatment, the therapy can “learn” by deliberately making small changes to the initial ideal EMF exposure to find a true, optimized solution. Real-time and intermittent sensing is inherently required for “learning” and consistent application of the treatment and will dynamically account for sensed distortion or attenuation of the ideal EMF.

The present invention provides a bioelectromagnetic treatment method for delivering personalized, effective bioelectromagnetic therapy to a microenvironment injury/disease site of a patient undergoing the therapy. The bioelectromagnetic treatment method is self-adaptive and bio-responsive to the microenvironment undergoing treatment and to the device administering therapy. No single electromagnetic field specification will work effectively for everyone. Novel computer-implemented computational engines process parameters defining a patient and injury to compute an ideal personalized electromagnetic field. The microenvironment computational engine calculates a personalized stimulation target using patient-specific factors and the macrotranslation computational engine incorporates external macro parameters related to device deployment to create an effective personalized EMF treatment protocol to achieve the calculated stimulation target.

The methods, device and system described herein minimizes undesired effects of electromagnetic fields on adjacent/non-target cells/tissue as electromagnetic field dispersion to adjacent tissues is minimized. The prescribed treatment modality in conjunction with the personalized treatment protocol maintains electromagnetic field concentration at the center of the microenvironment being treated.

In embodiments disclosed herein are methods, devices and systems for providing treatment of injury or disease in a patient by the application of personalized bioelectromagnetic therapy, including:
A patient-centric and microenvironment-centric Microenvironment Computational Engine (MiCE) configuredfor determination of an exact personalized microenvironment stimulation target (PMST);
An EMF-centric Macrotranslation Computational Engine configured for determining a personalized treatment protocol based on the PMST.

The computer implemented platform in conjunction with an EMF device provides personalized, intelligent, optimized and self-adaptive bioelectromagnetic therapy developed for the treatment of any injury and/or disease in a patient resulting in a better outcome for the patient.

Patient-based biophysical/biological ‘microenvironment parameters’ and EMF source deployment and transmission ‘macrotranslation parameters’ need to be accommodated on a personalized basis in the effective and successful operation of the bioelectromagnetic therapy. These patient-specific factors ultimately affect how an EMF elicits the required biological response at the microenvironment of the injury or disease of the patient.

Mathematical computation using the microenvironment computational engine described herein provides a means to calculate a patient-specific and theoretically ideal electromagnetic stimulation, i.e. PMST, for the microenvironment in need of treatment. The MaCE described herein computes the PTP that configures the initial settings of the EMF source to generate the PMST for the patient's microenvironment.

MaCE is configured to create a PTP based on the device deployment specifics and the transmission pathway. Following the PTP, the EMF source delivers an appropriate signal to generate the unique PMST. When calculating the personalized treatment protocol, accounting for and adapting to microenvironment and macrotranslation parameters unique to the individual enhances bio-effective processes involved in repair of injury and/or lessens and/or reverses progression of disease in a patient.

The personalized bioelectromagnetic therapy may additionally comprise an optional optimization extension that adaptively corrects/adjusts the treatment protocol in substantively real time to further optimize the personalized treatment protocol. The optimization extension includes two stages that follow after the calculation and delivery of the personalized treatment protocol and are designed to correct inaccuracies in the MiCE and MaCE configuration of outputs using a series of sensors. Optimization extension can be continuous or intermittent.

Optimization extension comprises two stages: (a) a feedback computational engine configured to acquire input data from EMF sensors at or near the microenvironment undergoing treatment and to determine and correct for differences between the target EMF defined by the MaCE and actual measured EMF, as a result of any inaccuracies in the MaCE and/or as a result of rotational movement and/or impact forces on the EMF modality; and (b) a learning computational engine configured to use the following inputs to operate a self-contained optimization loop: clinical sensor data obtained via one or more clinical sensors at or near the microenvironment, clinical follow-up input data and the adjusted treatment protocol.

The learning computational engine is configured for adjustment in small incremental changes to EMF source initial settings (e.g., increase or decrease the frequency by 10%) and for monitoring their effects using sensors. After mapping out the causal relationships, an optimized personalized treatment protocol is determined for continued treatment. The interim and final results of treatment and associated patient data and microenvironment data based on the personalized treatment protocol are relayed back into the MiCE. More specifically, real-time interim result data are fed back and integrated into one of the databases accessed by the microenvironment computational engine to improve the personalized microenvironment stimulation target for the current patient. The real-time inerim result data is cumulative during the treatment protocol such that there is successive addition to the patient data database.

The final result data are used to update the organized collection of clinical metadata to improve the determination of an effective microenvironment stimulation target for future patients.

The ability to logically and mathematically determine a personalized microenvironment stimulation target customized specifically to a target microenvironment of a patient is unique. Further, the ability to logically and mathematically integrate the personalized microenvironment stimulation target into the macrotranslation computational engine calculations to create a personalized treatment protocol is unique. The generation of an external EMF whose parameters, including waveform, frequency, amplitude, etc., are determined based on causal relationships between the EMF-derived stimulation and cellular responses, the specific attributes of the patient's injury, the specific attributes of the local microenvironment that are dependent on intrinsic patient characteristics, such as metabolic activity and limitations thereof, the transmission pathway between the EMF source and the microenvironment, and the specific configuration and operation of the EMF source induces the theoretically perfect electromagnetic field encompassing the cellular architecture of the microenvironment. This represents true customization of a bioelectromagnetic therapy for a specific patient with a specific biological challenge leading to an improved treatment outcome that is clinically effective and enduring.

### Personalized Bioelectromagnetic Device

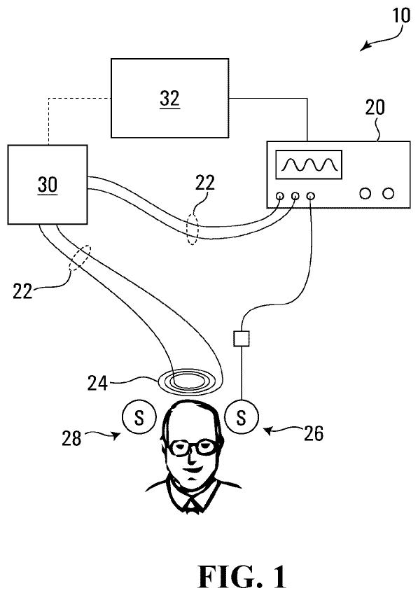

FIG. 1 illustrates basic components of a personalized bioelectromagnetic device (10) for delivery of personalized bioelectromagnetic stimulation therapy in accordance with the invention described herein to a target microenvironment. The device (10) comprises a signal generator (20) operationally connected via wires/leads (22) to send configured signals to the EMF source(s) (24) to deliver the specific and selective electrical signals. Operationally associated sensors are incorporated in the system, for example, EMF sensor(s) (26) for sensing deviations at or near the microenvironment between the personalized microenvironment stimulation target and the actual measured EMF and clinical sensor(s) (28) for sensing in vivo parameters relevant to the biological microenvironment undergoing treatment. An amplifier (30) is operationally connected to the output of the signal generator (20) to deliver the required input voltage signal to the EMF sources (e.g., active coils/electrodes). A power supply (32) may be directly connected to the signal generator, alternatively the signal generator may comprise a chargeable removable battery such as a lithium battery. In further embodiments, the device may feature a portable wireless power receiver for wireless transfer of power over distances.

In general, the EMF signal generator (20) is powered to output an electrical current with a defined waveform that flows into the coils or electrodes, creating the EMF. The device may transmit the personalized EMF signals as described herein via inductive coupling with a coil applicator or via capacitive coupling where the EMF applicators are electrodes in electrochemical contact with the conductive surface of the target for treatment.

A coil applicator comprises wire coils as loops of flexible wire. As part of a wearable bioelectromagnetic treatment device, in aspects, the coils are flexible/pliable and lightweight. As understood by one of skill in the art, the EMF source(s) may comprise a plurality or multiple coils/electrodes to deliver the personalized electromagnetic field to the target microenvironment. A multi-coil applicator may be made from a metal containing material such as a metal wire (e.g., copper) and coils of the applicator may be interconnected. One of skill in the art would understand that any desired number of coils or electrodes of various sizes and shapes can be incorporated depending on the mode of prescribed EMF treatment.

Sensors may be incorporated externally and/or implanted at the tissue level. It is understood by one skilled in the art that the number and type of sensor (EMF and/or) clinical may vary as can the positioning of these sensors with respect to the microenvironment targeted for treatment.

EMF sensors provide dynamic sensing of parameters at the microenvironment that may cause a deviation from the PMST. EMF sensors can comprise one or more of gaussmeters, magnetometer, spatial location sensors, force sensors, pressure sensors, shape sensing sensors, reversible and irreversible strain sensors and impedance sensors.

Clinical sensors provide dynamic sensing of biochemical parameters at the microenvironment and can comprise one or more of pH sensor, ion concentration sensor, glucose sensor, oxygen sensor, and temperature sensor, etc.

Safety sensors may be provided to alert the patient and the medical practitioner of any malfunction. These safety sensors operate to immediately shut down the device.

In embodiments, the system may be configured to limit the degree of adjustment to the treatment protocol by a patient in order not to manually expand the treatment protocol unless authorized by a clinical caretaker. In embodiments a patient may be able to self-direct changes to the therapy, but only for example between sessions or within an on-going session within predetermined limits programmed by the physician and/or device manufacturer. This prevents patient selection of a harmful treatment protocol operation.

Such limits or treatment governor functions may advantageously protect the patient from radically altering the therapy in an uncontrolled way that is very different from recent, familiar operating points. This may advantageously protect the patient from receiving an erratic course of therapies that may reduce the therapeutic value of the feedback on patient outcomes.

In embodiments the system is configured to govern the maximum increment of any parameter changes at the microenvironment.

In some embodiments, the system may send an electronic message or alert (for example email, text, call to a physician upon an attempt by a patient to expand the treatment protocol beyond that initialized in the system.

The device may comprise a housing with LCD or LED display to display information to the patient and may be a touch screen display. The housing may comprise a keypad for controls, and jack(s)/socket(s) for receiving the wire(s) (lead/harness) for connection to the EMF applicator or electrodes.

In some embodiments, the device and/or components thereof can be miniaturized for different configurations required in different treatment modalities.

The device may include one or more controllers/processors each including a central processing unit for processing data and computer-readable instructions and memory. The device may be configured to apply the personalized programmed treatment protocol. The steps of a method or a treatment protocol described in connection with the embodiments disclosed herein may be embodied directly in hardware, or in a software module executed by a processor. The memory may include volatile random-access memory (RAM), non-volatile read only memory (ROM), non-volatile magneto-resistive (MRAM) and/or other types of memory. The device may also include a data storage component for storing data and controller/processor-executable instructions. The data storage component may include one or more non-volatile storage types such as magnetic storage, optical storage, solid-state storage, etc. The device may also be connected to removable or external non-volatile memory and/or storage (such as a removable memory card, memory key drive, networked storage, etc.) through input/output device interface.

The device may be connected over any type of communication network implemented using wired infrastructure (e.g., cable, CATS, fiber optic cable, etc.), a wireless infrastructure (e.g., WiFi, RF, cellular, microwave, satellite, Bluetooth, etc.), and/or other connection technology. The device may connect to the network of the healthcare practitioner through either wired or wireless connections and further may include a local or private network and/or the internet. For example, the device may be connected to a network through a wireless service provider, over a WiFi connection or a cellular network connection. Network-connected support devices, such as a laptop computer, desktop computer, and a server may connect to the network through a wired or wireless connection.

Aspects of the system and disclosed herein may be implemented as a computer implemented method or as a device or a non-transitory computer readable storage medium (e.g. diskette, CD-ROM, ROM, or fixed disk) or interface device (via a medium to a network). The computer readable storage medium may be readable by a computer and may comprise computer executable instructions for causing a computer or other device to perform methods described in the present disclosure.

Some of the instructions that are executed during the execution of the method of the invention are described with reference to the operational flowcharts. Those skilled in the art will appreciate that the function, operation, determination, etc. of all or part of each step or combination of steps in the flowchart or block diagram may be implemented as computer program instructions, software, hardware, firmware, or combinations thereof. In addition, although the present invention may be embodied in software such as program code, the functions necessary to carry out the present invention are, optionally or alternatively, partially or wholly combined with combinational logic, firmware and or hardware components, such as application specific integrated circuits (ASICs), field programmable gate arrays (FPGAs) or other hardware, or some combination of hardware, software and/or firmware components.

Implementation of embodiments of the disclosed embodiments based on the flow charts and associated text description sufficiently sets forth the invention thus particular sets of program code instructions is not considered necessary for an adequate understanding of how to make and use embodiments.

The methods and systems described herein provide several computer advantages. The method efficiently and rapidly produces/transmits/retains personalized patient-based microenvironment target signals. The personalized treatment protocol and optional optimization extension can be accomplished in near real time.

### Interconnected Factors—Generation and Delivery of Emf for Producing A Desired Biological Response at the Microenvironment

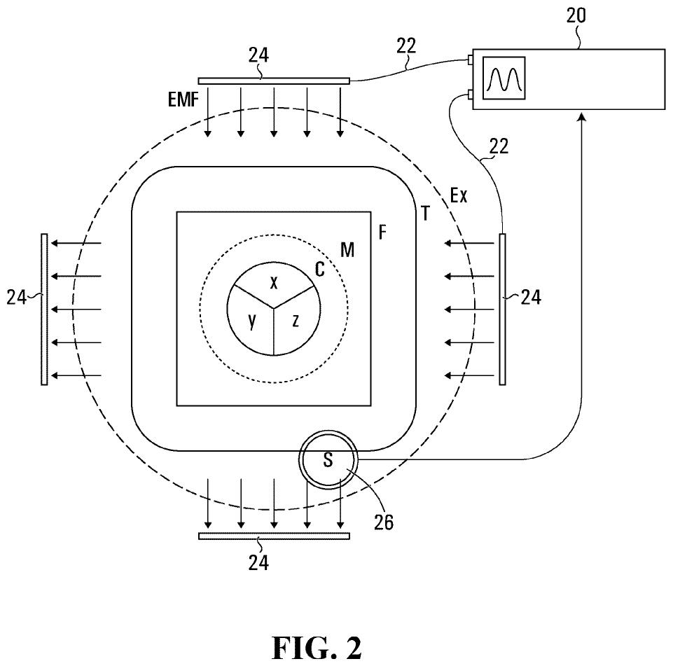

FIG. 2 is a representation of interconnected factors present at the microenvironment upon delivery of a theoretically ideal personalized microenvironment stimulation target signal.

A signal generator (20) of a device is shown operationally connected to EMF applicators (24) configured and positioned surrounding the target microenvironment (M). EMF sensors(S) installed externally or implanted at the tissue level can monitor the EMF induced at or near the microenvironment. The sensor data is fed into a feedback computational engine to continually compensate for any inaccuracies of the Macrotranslation Computational Engine, which defines EMF source output. Sensing of biological and functional progress may be done continuously or periodically and includes: biosensor dynamic sensing of biochemical parameters at the microenvironment, patient self-assessment (e.g. pain levels) and compliance, and one or more steps of clinical follow-up. The biological sensing data is used to determine if the biological response is achieved, as dictated by the Personalized Microenvironment Stimulation Target and is further incorporated to the proprietary clinical metadata.

Under control of the signal generator (20), the EMF applicators (24) are positioned to induce a focused field (F) that encompasses the microenvironment (M) to stimulate a biological response on at the cellular level (C). Such arrangement is to ensure the entire microenvironment is exposed to the electromagnetic field and cells within may experience the same magnetic forces and/or induced electric currents. With respect to a bone injury such as a fracture, exposure may enhance osteoblastic differentiation and promote bone healing. Additionally, EMF stimulation of a cancerous tumor can selectively accelerate apoptosis in cancer cells. At the cellular level an applied electromagnetic field disturbs the cellular membrane, for example forming lipidic nanopores for passage of ions, and may activate many intracellular pathways and physiological pathways. The mechanical action on both the intracellular and plasma_membrane levels, includes ion channels, receptors, cytokines, enzymes, and peripheral inflammatory pain modulators (Ross C L, et al., Altern Ther Health Med. 2016; 22:52-64). Cell behaviour in response to an induced EMF may be affected by a multitude of patient-specific factors (x, y, z).

The signal used to generate the field (F) is a function of: the microenvironment (M), both its physical location and the cellular (C) behaviour; the surrounding tissue (T); and external media (Ex) it may have to interact with. The tissue (T) level includes the electrical properties of the various tissues around the injury site, such as conductivity. External factors (Ex) encompass everything outside of the body, such as for example the device configuration (e.g., a hard support device such as a cast) and the configuration of the delivery system (e.g., size of coil and number of windings, number of coils), or simply an air gap.

The representation will be unique to the injury or disease of a patient due to the variability of the interconnected factors present at the microenvironment.

### Personalized Bioelectricity Treatment Protocol—Core Elements

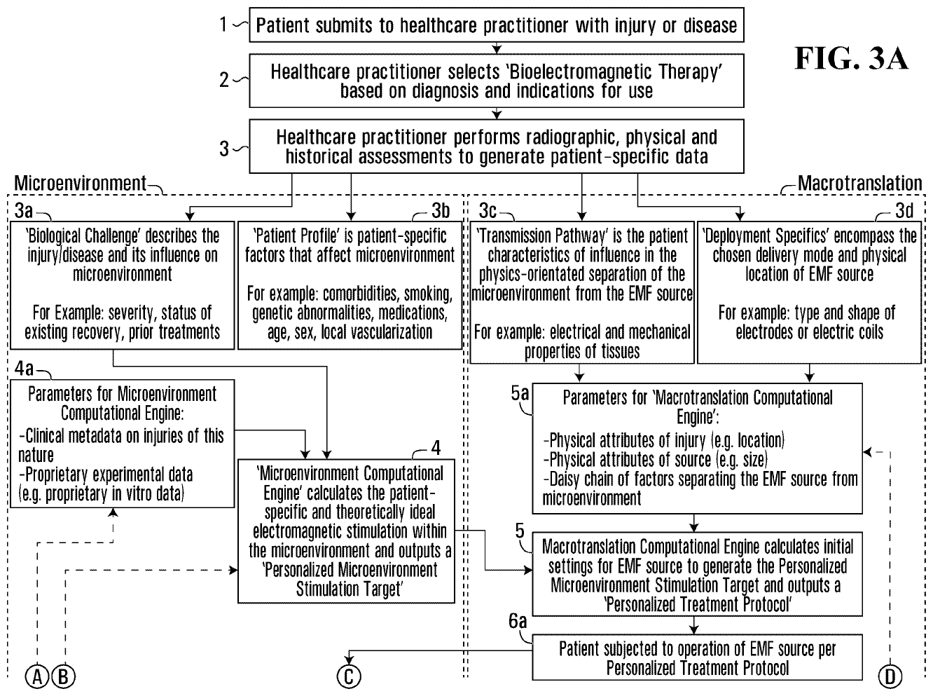

FIG. 3 is a therapy flowchart representing steps of the personalized stimulation therapy of the invention. The sequence is for use as a treatment of an injury or disease and further operationally applicable to clinical embodiments described herein. The reference numerals in [square brackets] below refer to the numbered sequence elements in FIG. 3.

A patient presents with an injury or disease [1] is diagnosed and subsequently recommended by a healthcare practitioner for Bioelectromagnetic Therapy (subject to relevant counterindications) [2]. The commercial implementation of Bioelectromagnetic Therapy is divided into components relating to the Microenvironment, which describes the factors influencing the injury/disease microenvironment, and Macrotranslation that includes details pertaining to the macroenvironment surrounding the microenvironment, and the electromagnetic field-producing device itself.

Prior to application of a therapy, the location of the injury or diseased tissue target volume and the relative locations and types of normal tissues are determined by diagnostic imaging and other medical techniques as would be understood by one of skill in the art. Using this information, and, optionally methods similar to those employed in radiation therapy planning, external (fiducial) marks can be placed on the patient surface as an anatomical marker to provide a reference coordinate system for targeting the injury or diseased tissue target volume within the body. Additionally, physical assessments and an analysis of the patient's historical medical circumstance are performed [3] to generate the following organized collection of data, which will be used to inform physics-based computational engines:
‘Biological Challenge’—this is data related to the unique detail of the patient's injury or disease, beyond just the type of injury/disease, which could have an effect on the microenvironment, such as for example, the severity, the current state of recovery, or the success or failure of prior treatments [3a];
‘Patient Profile’—this is data describing overall patient characteristics and conditions, and how such conditions influence the microenvironment. Demographics such as age, sex, height and weight are included, as well as a patient's comorbidities, smoking status, current medications, or existing medical conditions [3b];
‘Transmission Pathway’—the pathway separating the microenvironment and the EMF source is influenced by the material properties and physical dimensions of the tissues and materials along the signal path. These path-dependent properties affect the delivery of the required stimulating signal [3c];
‘Deployment Specifics’—this describes the mode of electromagnetic field generation and the physical construction of the device, as configured to the individual patient and that patient's injury/disease [3d].

All of the patient-specific information data is fed into a series of physics-based computational engines capable of calculating an effective treatment plan and optimizing such plan in relationship to the patient and the specific microenvironment of the patient being treated.

First, the ‘Microenvironment Computational Engine’ utilizes physics-based algorithms in combination with the patient-specific Biological Challenge and Patient Profile to compute a theoretically ideal ‘Personalized Microenvironment Stimulation Target’ [4]. The microenvironment computational engine also incorporates data from a database of clinical metadata and proprietary experimental data that is continually updated as treatments are optimized and concluded [4a]. These additional parameters aid in calculating an initial Personalized Microenvironment Stimulation Target by considering data from similar patients, similar injury and/or similar disease.

The Personalized Microenvironment Stimulation Target is sent to the ‘Macrotranslation Computational Engine’ [5] where macro parameters, such as location and size of the injury/disease and device, and the ‘daisy chain’ of material properties separating the device and the microenvironment [5a], are used to compute a ‘Personalized Treatment Protocol’. The Personalized Treatment Protocol describes the initial settings (i.e., frequency, intensity and waveform of the signal sent to the active EMF-generating elements) required to achieve the Personalized Microenvironment Stimulation Target at the microenvironment. Additionally, the Treatment Protocol includes the daily exposure and expected treatment length.

The patient receives the Personalized Treatment Protocol [6a] based on patient compliance with recommended therapy and thereby the patient microenvironment ideally receives electromagnetic stimulation that is exactly the Personalized Microenvironment Stimulation Target [6b].

Optionally, the treatment comprises an Optimization Extension comprising two further computational engines that obtain information from a series of sensors at the micro and macro levels to provide feedback (dashed connector lines) to optimize the Microenvironment Computational Engine and the Macrotranslation Computational Engine.

The first optional optimization engine is a ‘Feedback Computational Engine’ [7] that takes input from EMF sensors [7a] at or near the microenvironment to determine and correct for differences between the Microenvironment Stimulation Target and the actual measured EMF, as a result of inaccuracies in the Macrotranslation Computational Engine. Any corrections are sent as feedback to the parameters influencing the calculation of the Personalized Treatment Protocol.

Throughout treatment, progress data is collected via clinical follow-up [8a] and clinical sensors [8b], including any updated clinical assessments including for example radiographic or functional assessments, biomarker assays, real-time biosensors, compliance metrics, or input directly from the patient (e.g., pain scores and/or compliance scores on a visual analogue scale).

The ‘Learn Computational Engine’ [9] provides a final round of optimization during the active treatment, via a self-contained feedback loop, using inputs from follow-up and/or sensor data. The Treatment Protocol is optimized to compensate for inaccuracies in the Microenvironment Computational Engine through the following sequence:
i. Incrementally adjust individual parameters of the Treatment Protocol (e.g., increase and decrease the frequency or intensity of stimulation by about 5%);
ii. Monitor the effect of any small changes;
iii. Collect sensor data and map a patient-specific response profile for each parameter; and
iv. Select the optimal combination of settings for the patient's recovery and output an optimized Treatment Protocol.

Interim and final results of the Bioelectromagnetic Therapy, including all associated patient information, are fed back into the system to improve treatment for both the current patient and future patients, respectively [10]. Interim results [10a] are fed back into the Microenvironment Computational Engine to improve the current Stimulation Target. The final results [10b] are used to update the collection of clinical metadata used to calculate a personalized microenvironment stimulation target for future patients.

### Embodiments—Bioelectromagnetic Treatment Plans

These embodiments are provided for purposes of illustration only and are not intended to be limiting unless otherwise specified. Thus, the invention should in no way be construed as being limited by the following studies, but rather, should be construed to encompass any and all variations which become evident as a result of the teaching provided herein.

### Personalized Bioelectricity Treatment Protocol for Bone

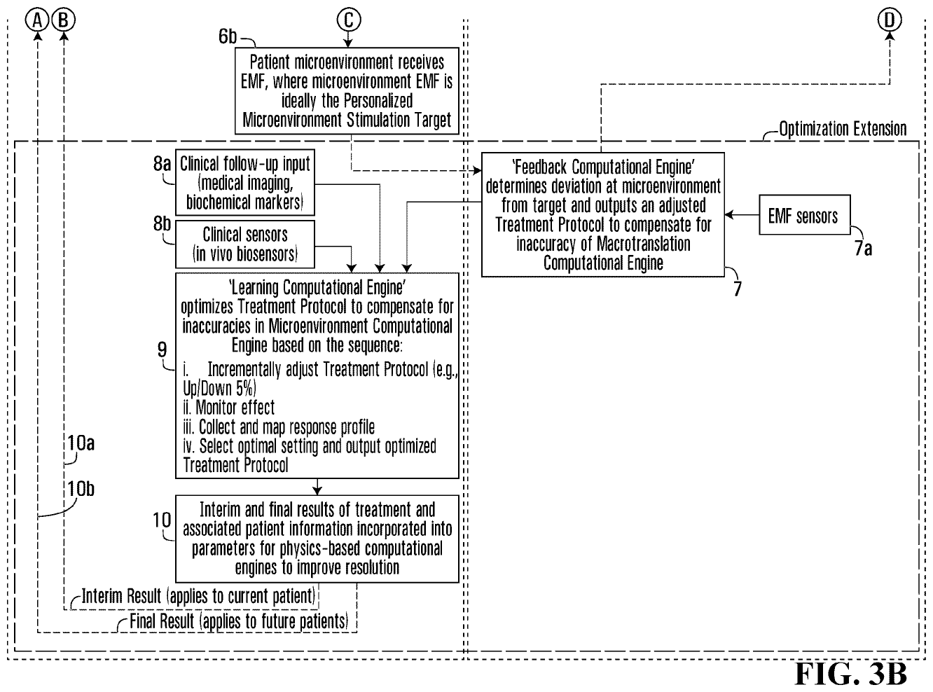

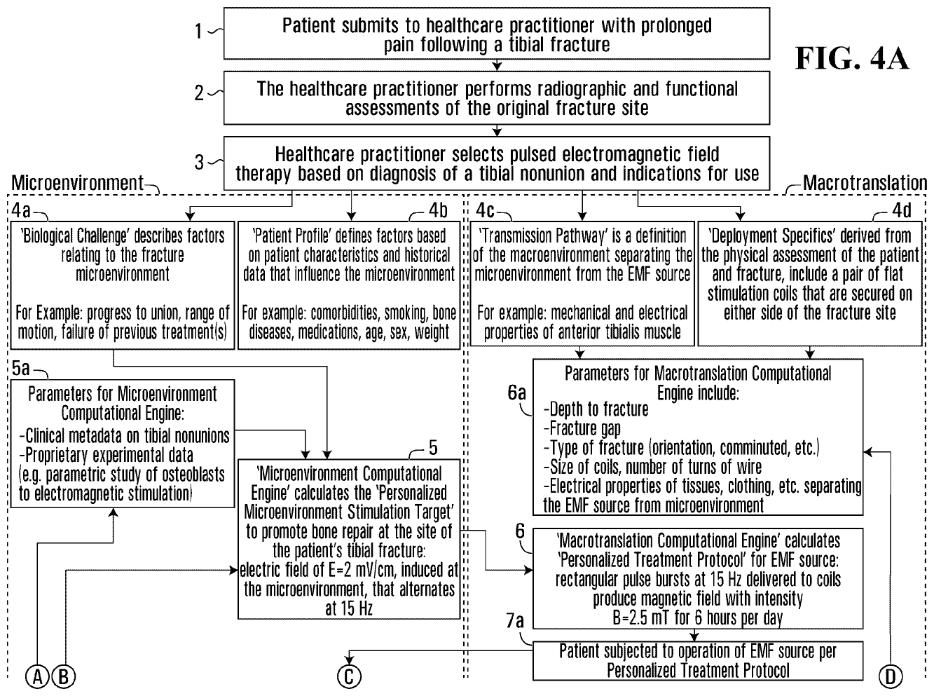

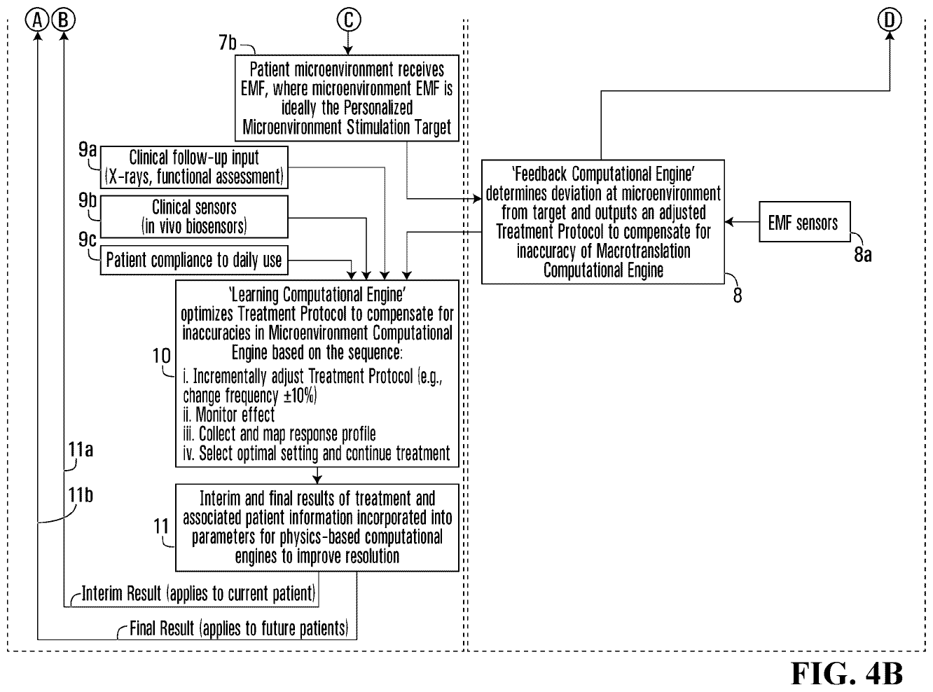

FIGS. 4(A) and 4(B) show a therapy flowchart representing steps of the personalized stimulation therapy of the invention from diagnosis through end of treatment for treatment of a bone fracture. As a representative non-limiting clinical example, the protocol is presented with respect to a non-union fracture. The reference numerals below refer to the numbered sequence elements in FIGS. 4(A) and (B).
1. Symptoms: A patient submits to a healthcare practitioner with pain due to a tibial fracture months earlier that has persisted despite prior treatment(s).
2. Diagnosis: The healthcare practitioner assesses the fracture (radiographic and functional) and diagnoses the patient with a nonunion fracture.
3. Recommend PEMF therapy: The healthcare practitioner recommends Bioelectromagnetic Therapy using a non-invasive and personalized (patient-specific) Pulsed Electromagnetic Field (PEMF) therapy device to promote the body's natural healing processes. Indications for use include a fracture gap less than 5 mm, no synovial fluid, mechanical alignment, no significant atrophy, etc.
4. Patient data collection: The practitioner collects patient historical data and interprets their clinical assessments to define the following:
   a. Injury specifics, termed ‘Biological Challenge’, are gathered based on X-rays and a functional assessment of the fracture that provide a definition of the microenvironment and its treatment history [4a];
   b. Patient Profile, including age, sex, body-mass index, smoking, comorbidities, etc., that may affect the microenvironment and its susceptibility to electromagnetic stimulation [4b];
   c. The Transmission Pathway, which is a description of the tissues and materials (and their mechanical and electrical properties) that separate the microenvironment from the EMF source [4c];
   d. Deployment Specifics include the healthcare practitioner's selection of the mode of electromagnetic therapy and the physical description of the device and how it will be coupled to the patient [4d].
5. Prescribe a Personalized Microenvironment Stimulation Target: The Microenvironment Computational Engine computes an electromagnetic target for effective stimulation of bone repair at the fracture site by incorporating data from the Biological Challenge [4a] and Patient Profile [4b], as well as the following parameters:
   a. Clinical metadata on tibial nonunions (collected from prior clinical trials or literature, and from previous patients) and proprietary experimental data, which includes animal study data or in vitro results (e.g., effects of PEMF on osteoblasts) [5a].
These parameters [5a] are continually updated and are primarily used to estimate a range for the Personalized Microenvironment Stimulation Target, while 4a and 4b are used to produce a target that is truly personalized to the patient and their injury.
6. Compute the Personalized Treatment Protocol: The Macrotranslation Computational Engine computes the EMF source signal parameters (amplitude, frequency, waveform, etc.) that must be delivered to the therapeutic coils to produce the desired Stimulation Target at the microenvironment. Additionally, the calculation is performed by accounting for the following:
   a. Parameters that physically describe the factors in between the microenvironment and the device, such as the depth to fracture, the size and shape of the treatment coils, and the conductivity and/or attenuation of muscle, bone or fabrics (to name a few) that the EMF may interact with. These factors are directly derived from 5c and 5d.
7. Begin treatment as per personalized treatment protocol:
   a. The patient wears the prescribed active treatment device for 6 hours per day over a period of at least six months [7a];
   b. The microenvironment receives stimulation that ideally matches the Microenvironment Stimulation Target [7b].
8. EMF sensors [8b] at or near the microenvironment detect differences between target stimulation and actual electromagnetic stimulation: A Feedback Computational Engine takes input from EMF sensors [8b] and sends feedback to update the parameters for Macrotranslation Computational Engine to compensate for differences between the Personalized Microenvironment Stimulation Target and the actual electromagnetic quantities detected by the sensors. This feedback occurs for the current patient.
9. Data is collected reflecting progress to union: Information is collected during treatment using the following methods:
   a. Clinical follow-up—X-rays and functional assessments are performed and compared to previous results. Additionally, blood and serum tests are performed to measure expression of biochemical factors at the microenvironment;
   b. Clinical sensors—Any form of in vivo biosensor;
   c. Patient compliance—The device tracks compliance via daily exposure time.
10. Learn and optimize the personalized treatment protocol: To compensate for inaccuracies in Microenvironment computational Engine, the Learn Computational Engine accepts sensor data and the current Treatment Protocol and executes a self-contained feedback loop involving the following steps:
   i. Incrementally adjust the EMF source parameters (+5-10%) such as the frequency, driving voltage or waveform shape;
   ii. Monitor the effect of each change (via sensors);
   iii. Collect data and map out a response profile by repeating these three steps;
   iv. Select the optimal combination of settings to continue treatment.
11. Results are used as feedback for current and future patients:
   a. Interim results act as feedback for current patient. Any further follow-up and progress after optimization are used as feedback to the microenvironment computational engine in order to further refine the Personalized Microenvironment Stimulation Target [11a];
   b. Final results are used to update metadata. Upon successful completion of treatment (union has been achieved), the Personalized Stimulation Target, Treatment Protocol and all patient-specific data is used to update the clinical metadata for tibial nonunion fractures, which will, in turn, be incorporated into the computational physics engines for future patients [11b].

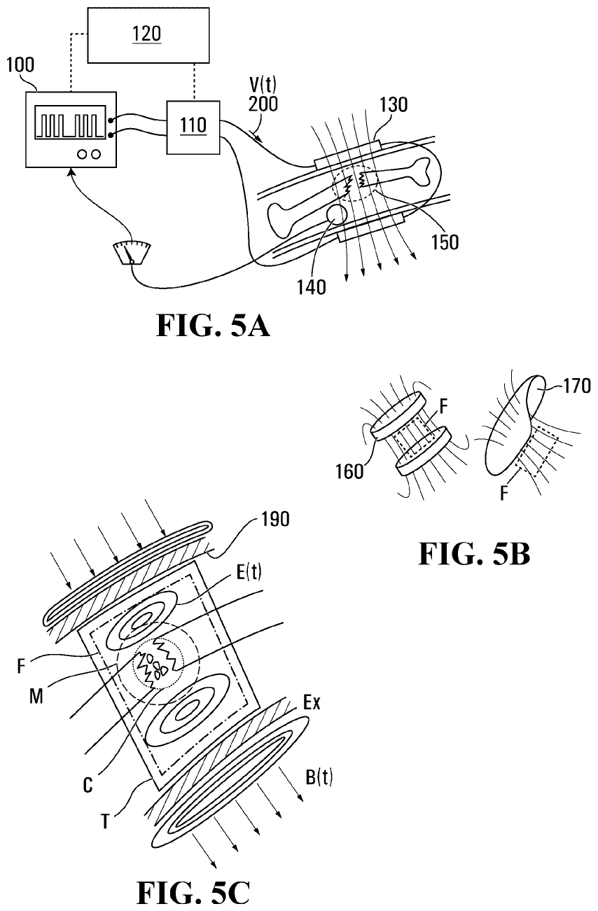

The prescribed PEMF device configuration is shown in FIGS. 5(A) and 5(B). FIG. 5(A) shows the signal generator (100) that controls the EMF sources (130) (i.e. coils) positioned to induce a focused field (F) that encompasses the microenvironment (M) to stimulate a biological response on at the cellular level (C). The EMF device components, power supply (120), amplifier (110), and signal generator (100) are operationally connected via wires/leads to the positioned EMF sources (130) each substantially adjacent the fracture and sensors (140). The hardware are positioned in order that the personalized EMF stimulation signal encompasses the fracture area (150). FIG. 5(B) shows two configurations of the EMF applicators (130), either as parallel coils (160) or shaped coil (170) contoured to the placement area for treatment. FIG. 5(C) is an enlarged schematic of the tibial nonunion fracture microenvironment (M) showing delivery of the theoretically ideal PMST signal and how that may affect interconnected factors present at the microenvironment of the fracture similar to that explained with respect to FIG. 2.

Example 1 presents experimental data demonstrating variability in the response of cells from a specific donor and between different donors to different EMF stimulation. This supports the need for the described personalized bioelectricity treatment protocol for treatment of bone as shown with non-union fracture healing.

### Personalized Bioelectricity Treatment Protocol—Cancer

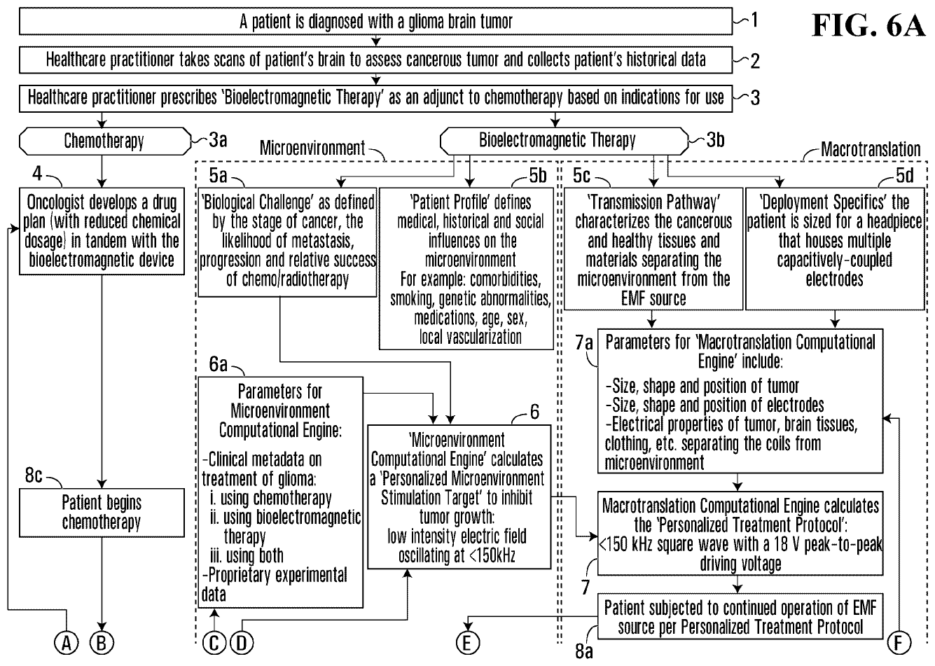

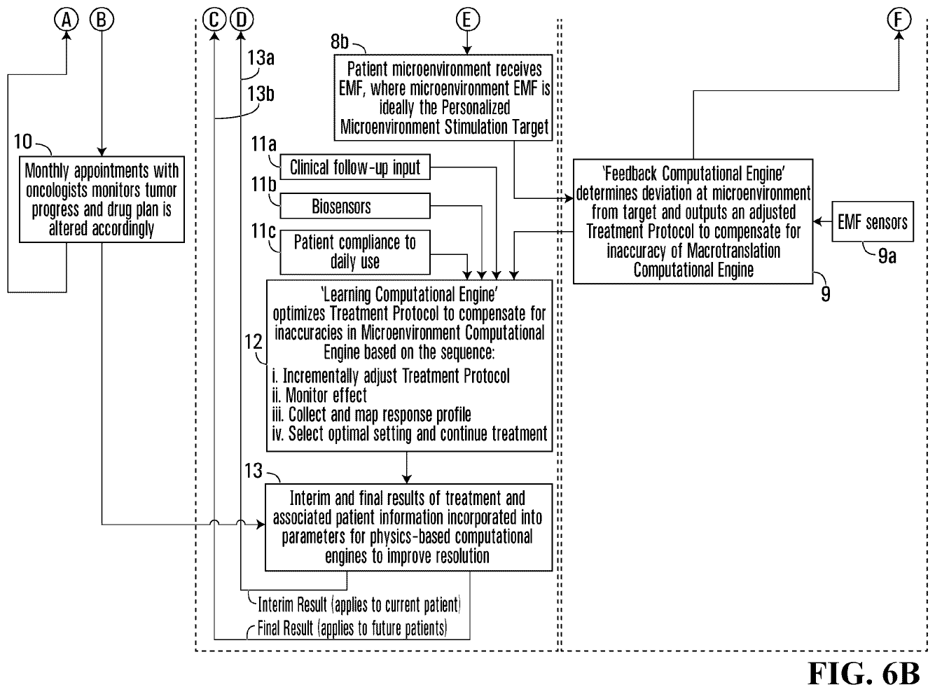

FIG. 6 shows a therapy flowchart representing steps of the personalized stimulation therapy of the invention from diagnosis through end of treatment for treatment of cancer. The personalized electrical stimulation is applied to a cancerous tumor as a form of adjuvant treatment combined with an appropriate chemotherapy plan. As a representative non-limiting example of a clinical embodiment of cancer, the protocol is presented with respect to a glioma.

The reference numerals below refer to the numbered sequence elements in FIG. 6.
1. Diagnosis: The patient is diagnosed with a glioma brain tumor that requires treatment to stop the rapid growth of cancerous cells.
2. Assessment and indications for electrical stimulation: The physician takes scans of the affected tissue and reviews any prior oncological treatment history when considering eligibility for bioelectromagnetic stimulation therapy.
3. Prescribe Bioelectromagnetic Therapy as adjuvant: The healthcare practitioner recommends electromagnetic field stimulation (using a personalized, capacitively-coupled bioelectromagnetic therapy device) as an adjuvant to chemotherapy.
   a. In combination, the chemotherapy dosage can be reduced relative to chemotherapy alone and mitigates adverse side effects [3a];
   b. Bioelectromagnetic therapy is specifically targeted at cancer cells within the tumor with the aim of triggering apoptosis (cell death) [3b].
4. Chemotherapy drug plan: An oncologist develops a drug plan focused on attacking the cancerous tumor.
5. Patient data collection for Bioelectromagnetic Therapy: The practitioner uses clinical and historical assessments to define the following:
   a. Biological Challenge data includes the stage of the cancer, the likelihood/degree of metastasis, and the relative success of prior chemo/radiotherapy treatments [5a];
   b. Patient Profile, including age, sex, body-mass index, smoking, comorbidities, etc., that may affect the micro-environment and its susceptibility to electromagnetic stimulation [5b];
   c. The Transmission Pathway describes the physical size and position of the tumor as well as the electrical properties of both cancerous and healthy tissues separating the microenvironment and the stimulatory electrodes [5c];
   d. Deployment Specifics—the active electrodes are built into a headpiece that maintains field focus and can be worn comfortably all day [5d].
6. Calculate a Personalized Microenvironment Stimulation Target: The Microenvironment Computational Engine is used to compute the initial stimulation target required to interrupt the division of tumor-specific cells. Stimulation is delivered via a low-intensity electric field induced in the tumor that alternates at <150 kHz.
The engine incorporates data from the Biological Challenge [5a] and Patient Profile [5b], as well as the following parameters:
   a. Clinical metadata on the treatment of glioma with chemotherapy and/or bioelectromagnetic therapy, and proprietary experimental data [6a].
7. Compute the Personalized Treatment Protocol: The Macrotranslation Computational Engine calculates the signal required to meet the Stimulation Target to be a <150 kHz square wave with an 18 V peak-to-peak driving voltage. The Treatment Protocol is considered to be continued indefinitely and until adequate progress has been identified by the healthcare practitioner. The calculation is performed by accounting for the following:
   a. The electrical properties of tumor and brain tissue, and the size, shape and position of both the tumor and the electrodes [7a].
8. Begin treatment as per Personalized Treatment Protocol and physiotherapy regimen:
   a. The patient wears the headpiece during every-day activities and while sleeping [8a];
   b. The microenvironment receives stimulation that ideally matches the stimulation target [8b];
   c. The patient begins chemotherapy [8c].
9. EMF sensors [9a] at or near the microenvironment detect differences between target stimulation and that delivered by the Treatment Protocol: The Feedback Computational Engine takes input from EMF sensors [9a] and sends feedback to update the parameters for the Macrotranslation Computational Engine to compensate for differences between the Personalized Microenvironment Stimulation Target and the actual values detected by the sensors. This feedback occurs for the current patient.
10. Regular follow-up with oncologist: Every month, the patient is reassessed by an oncologist and updates are made to their drug plan as necessary.
11. Sensor data collection and follow-up assessments: Information is collected during treatment using the following methods:
   a Clinical follow-up-brain scans of tumor are compared to previous results [11a];
   b. Clinical sensors-Any form of in vivo biosensor [11b];
   c. Patient compliance—The device tracks compliance via daily active exposure time [11c].
12. Learn and optimize the Personalized Treatment Protocol: The Learn Computational Engine compensates for inaccuracies in the microenvironment computational engine using sensor data and the current Treatment Protocol and executes a self-contained feedback loop involving the following steps:
   i. Incrementally adjust the EMF source parameters (+5-10%);
   ii. Monitor the effect of each change (via sensors);
   iii. Collect data and map out a response profile by repeating these first three steps;
   iv. Select the optimal combination of settings to continue treatment.
13. Results are used as feedback for current and future patients:
   a. Interim results act as feedback for current patient. Any further follow-up and progress after optimization are used as feedback to the Microenvironment Computational Engine in order to further refine the Personalized Microenvironment Stimulation Target [13a];
   b. Final results are used to update metadata. Upon successful completion of treatment, the personalized Stimulation Target, Treatment Protocol, chemotherapy plan, and all patient-specific data is used to update the clinical metadata for glioma treatment, which will, in turn, be incorporated into the physics-based computational engines for future patients [13b].

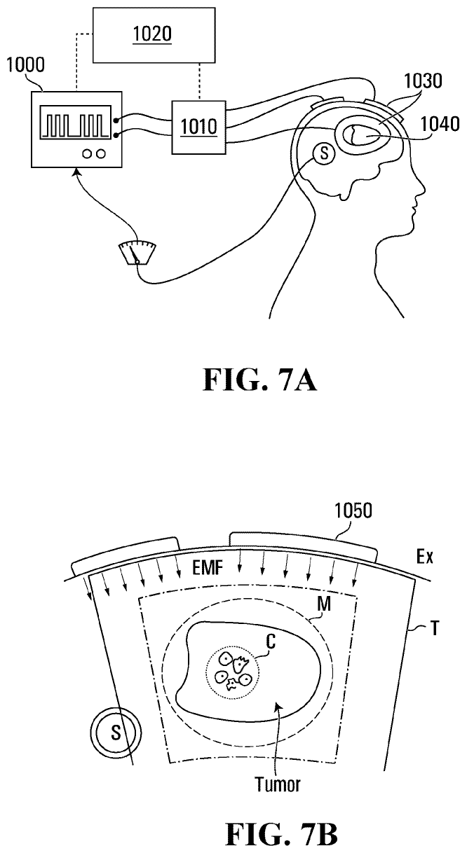

The prescribed capacitive-coupled EMF device configuration is shown in FIGS. 7A-7B. FIG. 7A shows the EMF device components and positioning of the EMF applicators substantially adjacent the tumor in order that personalized EMF stimulation encompasses the tumor. FIG. 7B is an enlarged schematic of the tumor microenvironment showing delivery of the theoretically ideal PMST signal and how it may affect interconnected biological factors present at the microenvironment of the tumor similar to that explained with respect to FIG. 2.

Experimental data presented in Example 2 demonstrates that EMF therapy inhibit cell growth of breast cancer cells and also enhances chemotherapeutic effects. This confirms the need for the described personalized bioelectricity treatment protocol related to cancer therapy.

### Personalized Bioelectricity Treatment Protocol for Pain Management

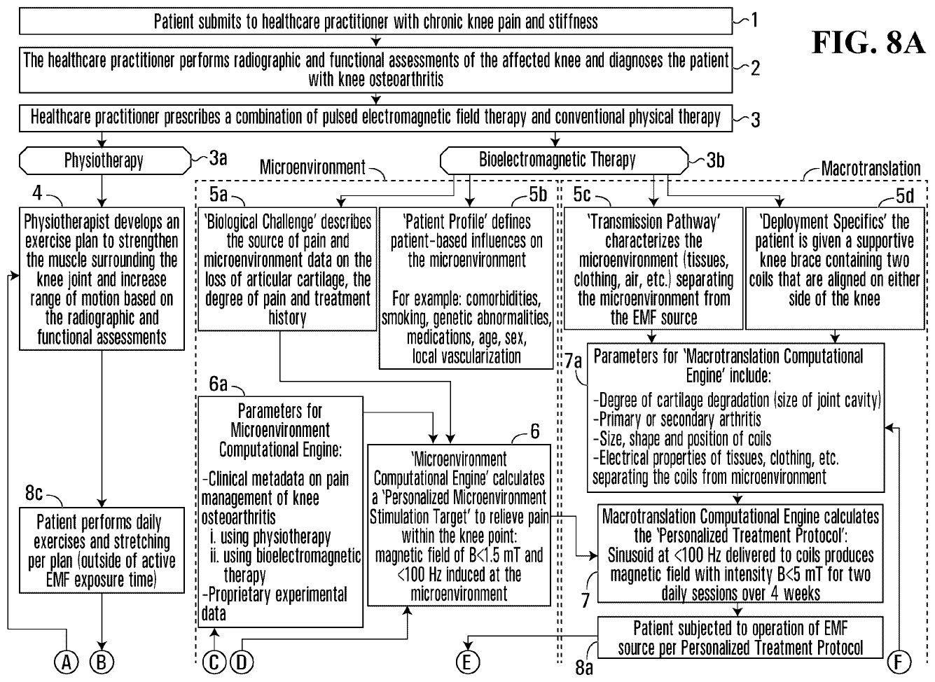

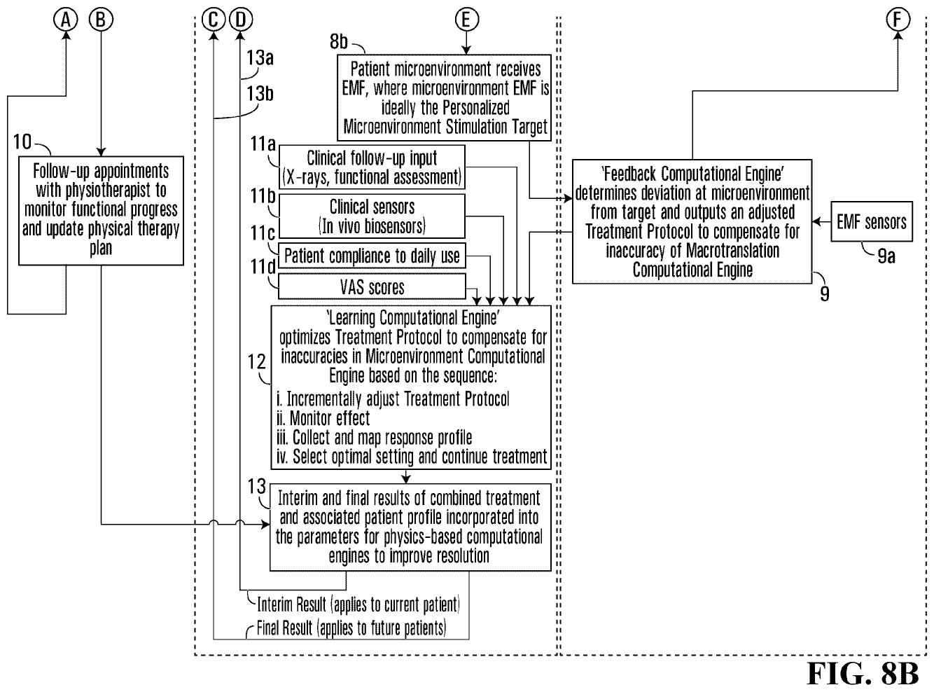

FIG. 8 shows a therapy flowchart representing steps of the personalized stimulation therapy of the invention from diagnosis through end of treatment for the promotion of pain relief. As a representative non-limiting example of a clinical embodiment of pain relief, the protocol is presented with respect to chronic pain due to osteoarthritis of the knee. The reference numerals below refer to the numbered sequence elements in FIG. 8.
1. Chronic knee pain: A patient presents with pain and stiffness in the knee joint accompanying movement or long periods of inactivity.
2. Diagnosis: The healthcare practitioner takes X-rays of the afflicted knee, performs a functional assessment, and diagnoses the patient with knee osteoarthritis.
3. Prescribe combination of physiotherapy and Bioelectromagnetic Therapy: The healthcare practitioner recommends pulsed electromagnetic field stimulation (using a personalized bioelectromagnetic therapy device) as an adjunct to conventional physical therapy.
   a. Physiotherapy aims to strengthen tissues surrounding the joint to relieve stiffness and improve range of motion [3a];
   b. Bioelectromagnetic therapy targets the osteoarthritic microenvironment to promote cellular activity that relieves pain [3b].
4. Physical therapy regimen: A physiotherapist, with reference to the radiographic results and their own assessments, develops an exercise and stretching plan centered around strengthening the muscles groups around the knee joint and increasing range of motion.
5. Patient data collection for Bioelectromagnetic Therapy: The practitioner uses patient historical data and the physical and radiographic assessments to define the following:
   a. Biological Challenge data includes the degree of articular cartilage degeneration, pain measured on a visual analogue scale (VAS), treatment history, etc. [5a];
   b. Patient Profile, including age, sex, body-mass index, smoking, comorbidities, etc., that may affect the micro-environment and its susceptibility to electromagnetic stimulation [5b];
   c. The Transmission Pathway defines the electrical properties and relative position/shape of the cartilage, bone, muscles and tendons surrounding the microenvironment [5c];
   d. Deployment Specifics—the circular/elliptical stimulation coils are built into a supportive knee brace that positions one coil on either side of the knee joint [5d].
6. Calculate a Personalized Microenvironment Stimulation Target: The Microenvironment Computational Engine is used to compute the initial Stimulation Target aimed at relieving the chronic symptoms of knee osteoarthritis. Stimulation is delivered via frequencies <100 Hz and magnetic flux densities B<1.5 mT induced within the microenvironment.
The Engine incorporates data from the Biological Challenge [5a] and Patient Profile [5b], as well as the following parameters:
   a. Clinical metadata on knee osteoarthritis and proprietary experimental data, which includes animal study data or in vitro results (e.g., down-regulation of interleukin-1ß in vitro) [6a].
7. Compute the Personalized Treatment Protocol: The Macrotranslation Computational Engine calculates the signal required to match the Microenvironment Stimulation Target to be a<100 Hz sinusoid with B<5 mT. The Treatment Protocol requires the device be activated twice a day for 1 hour each for at least 4 weeks. The calculation is performed by accounting for the following:
   a. The electrical properties of interacting media, size of joint cavity, and the size, shape and position of the coils [7a].
8. Begin treatment as per Personalized Treatment Protocol and physiotherapy regimen:
   a. The patient wears the supportive brace whenever possible and is subjected to two daily active sessions [8a];
   b. The microenvironment receives stimulation that ideally matches the Stimulation Target [8b];
   c. The patient performs daily exercises and stretches as per the physiotherapist's recommendation [8c].
9. EMF sensors [9a] at or near the microenvironment detect differences between target stimulation and that delivered by the Treatment Protocol: The Feedback Computational Engine takes input from EMF sensors [9a] and sends feedback to update the parameters for the Macrotranslation Computational Engine to compensate for differences between the Personalized Microenvironment Stimulation Target and the actual values detected by the sensors. This feedback occurs for the current patient.
10. Regular physiotherapy appointments: Every two weeks, the patient visits the physiotherapist, and the physical therapy plan may be updated for the subsequent two-week period.
11. Sensor data collection and follow-up assessments: Information is collected during treatment using the following methods:
   a. Clinical follow-up-X-rays and functional assessments are performed and compared to previous results [11a];
   b. Clinical sensors-Any form of in vivo biosensor [11b];
   c. Patient compliance—The device tracks compliance via daily active exposure time [11c];
   d. Daily VAS score-Before and after each set of two daily sessions of EMF exposure, the patient should record their pain levels using a VAS score. Data is recorded using a user-interface via an internet-connected mobile device [11d].
12. Learn and optimize the Personalized Treatment Protocol: The Learn Computational Engine compensates for inaccuracies in the Microenvironment Computational Engine using sensor data and the current Treatment Protocol and executes a self-contained feedback loop involving the following steps:
   i. Incrementally adjust the EMF source parameters (+5-10%);
   ii. Monitor the effect of each change (via sensors);
   iii. Collect data and map out a response profile by repeating these first three steps;
   iv. Select the optimal combination of settings to continue treatment.
13. Results are used as feedback for current and future patients:
   a. Interim results act as feedback for current patient. Any further follow-up and progress after optimization are used as feedback to the Microenvironment Computational Engine in order to further refine the Personalized Microenvironment Stimulation Target [13a];
   b. Final results are used to update metadata. Upon successful completion of treatment, the Personalized Stimulation Target, Treatment Protocol, physiotherapy progress, and all patient-specific data is used to update the clinical metadata for knee osteoarthritis-related pain, which will, in turn, be incorporated into the physics-based computational engines for future patients [13b].

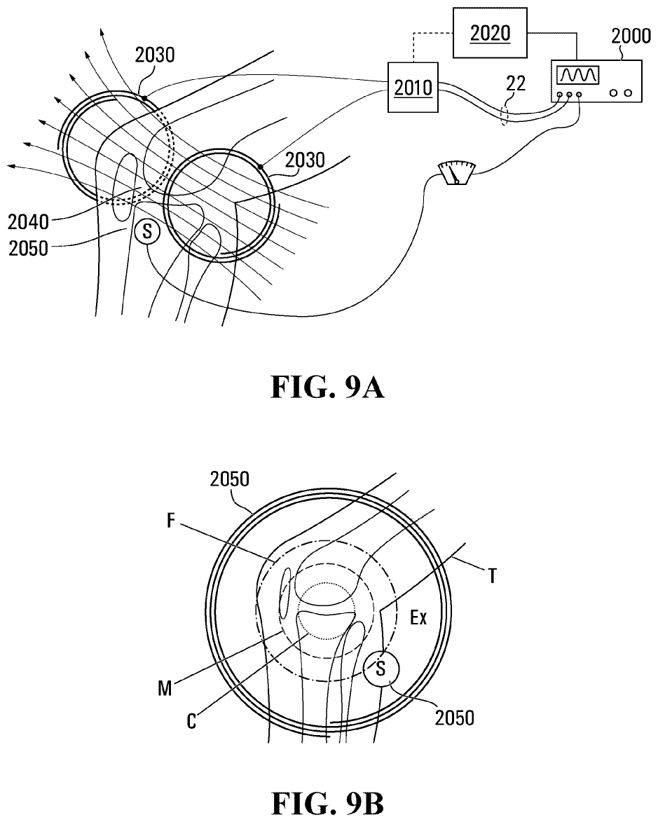

The prescribed PEMF device configuration is shown in FIGS. 9A-9B. FIG. 9A shows EMF device components and positioning of the EMF applicators substantially adjacent the knee joint in order that personalized EMF stimulation encompasses the source of pain. FIG. 9B is an enlarged schematic of the knee joint microenvironment showing delivery of the theoretically ideal PMST signal and how it may affect interconnected biological factors present at the microenvironment of the knee joint similar to that explained with respect to FIG. 2.

Experimental data shown in Example 3 demonstrates inter-donor and intra-donor variability in pain-related gene expression in human astrocytes associated with different EMF stimulation profiles. This confirms the need for the described personalized bioelectricity treatment protocol for to pain management.

### Scope of Treatment Methodologies

The personalized bioelectromagnetic therapy method, device and system described herein are suitable for non-invasive or invasive treatment of injury or disease and advantageously does not possess negative side effects of pharmacological treatments. It can, however, be used in conjunction with pharmacological treatments. Furthermore, it can be used before other treatments, after completing a different treatment approach or in conjunction with other therapeutic and prophylactic procedures and modalities such as heat, cold, ultrasound, wound dressing, orthopedic fixation devices, and surgical interventions.

Treatment of injury or disease may include but not be limited to cancer, cardiovascular disease, inflammatory disease, autoimmune disease, neurological disease, musculoskeletal pain management, wound repair, bone repair, osteoporosis, tissue repair, rehabilitation of traumatic injuries, sports injuries and surgical rehabilitation. The injury or disease is not limiting.

The method of the invention modulates physiologically relevant pathways of the targeted injury/disease microenvironment, such as general transmembrane potential changes, involved in stabilizing, reversing, healing of injury or disease. Some of the physiological induced changes may include variations in cell membrane, enzymatic activity, cell apoptosis, nerve conduction, collagen synthesis, vasodilation, vasoconstriction, viscosity of body fluids/blood, pain signaling, production of endorphins, tissue metabolism, inflammation, supply of oxygen & nutrients, tissue/muscle repair or healing, fibroblast activity, collagen fibril density, protein synthesis, and tissue regeneration.

The method of the invention also provides an improved means to enhance blood flow and biochemical activity by action of exogenous factors (for example growth factors and cytokines) to accelerate repair of cells, organs and tissues, and modulate angiogenesis and neovascularization.

The method of the invention may modulate activity of a variety of biochemical molecules/markers involved to promote healing of an injury or disease. Representative non-limiting examples include cytokines, growth factors, tumor markers, inflammatory markers, endocrine markers and metabolic markers.

Exemplary growth factors may include EGF ligands, EGF, TGFα, EGFR/ErbB receptor family, FGF family, IGF family, IGF-binding protein (IGFBP) family, receptor tyrosine kinases, proteoglycans, TGFβ super family and VEGF/PDGF family.

Exemplary inflammatory markers may include ICAM-1, RANTES, MIP-2, MIP-1β, MIP-1α, MMP-3, adhesion molecules, vitronectin, fibronectin, collagen, laminin, ICAM-1, ICAM-3, BL-CAM, LFA-2, VCAM-1, NCAM, PECAM, cytokines such as the IFN family, chemokines, tumor necrosis factor (TNF), TNF superfamily receptors and modulators, TGFβ, superfamily ligands BMP (bone morphogenetic protein), EGF ligands, fibrinogen, glial markers, (MHC) glycoproteins, microglial markers, α2 macroglobulin receptor, fibroblast growth factor, angiogenic factor-1, MIF, blood vessels Nascent factor-2, CD14, β-defensin 2, MMP-2, nitric oxide, endothelin-1, and VEGF.

Exemplary cytokines may include FGF basic, G-CSF, GCP-2, granulocyte macrophage colony stimulating factor GM-CSF (GM-CSF), growth-related oncogene-keratinocytes (GRO-KC), HGF, ICAM-1, IFN-α, IFN-γ, interleukins, interferon-inducible proteins, MCP-1, macrophage inflammatory proteins, tumor necrosis factor family, VCAM-1, and VEGF.

Exemplary tumor markers may include EGF, TNF-α, PSA, VEGF, TGF-β1, FGFb, TRAIL, and TNF-RI (p55).

Exemplary markers of endocrine function may include 17β-estradiol (E2), DHEA, ACTH, gastrin, and growth hormone (hGH).

Exemplary markers of autoimmune function may include GM-CSF, C-reactive protein, and G-CSF.

Exemplary cardiovascular markers may include cardiac troponin I, cardiac troponin T, brain natriuretic peptide, NT-proBNP, C-reactive protein HS, and β thromboglobulin.

Exemplary metabolic markers may include Bio-intact PTH (1-84) and PTH.

In certain embodiments, personalized bioelectromagnetic target signals may have an effect in vivo on stem cell homing signals (SDF-1 and PDGF), stem cell differentiation signals, blood vessel growth signals, and organ-specific tissue building signals.

In certain embodiments, the bioelectromagnetic target signals may have an effect in vivo on blood vessel growth factors, e.g., VEGF, SDF-1, PDGF, HIF 1 α, eNOS, tropoelastin, HGF, and EGF.

In certain embodiments, personalized bioelectromagnetic target signals may have an effect in vivo on for example SDF-1, IGF-1, HGF, EGF, PDGF, eNOS, VEGF, follistatin, Activin A and B, Relaxin, tropoelastin, GDF-10, GDF-11 and Neurogenin-3.

In certain embodiments, personalized bioelectromagnetic target signals may have an effect in vivo on a protein selected from the group consisting of SDF-1, IGF-1, HGF, EGF, PDGF, VEGF, HIF 1 alpha, eNOS, activin A, activin B, IL-6, follistatin, tropoelastin, GDF-10, GDF-11, neurogenin 3, FGF, TGF, TNF alpha, RANKL, OPG, and combinations thereof.

In certain embodiments, personalized bioelectromagnetic target signals may have an effect in vivo on activity of osteoblasts, osteocytes, osteoclasts, fibroblasts, chondrocytes, keratinocytes, endothelial cells, epithelial cells, mature macrophages and granulocytes.

In certain embodiments, personalized bioelectromagnetic target signals may have an effect in vivo to stimulate multipotent adult stem cells (mesenchymal stem cells or bone marrow stem cells) to promote proliferation and differentiation of the multipotent adult stem cells into specific pathways such as bone, connective tissues, fat etc.

In certain embodiments, the bioelectromagnetic methods according to the present disclosure may be applied for treatment of osteoarthritis and associated pain and/or inflammation in peripheral structures, such as an inflamed knee joint. Neurochemical and metabolic changes in the area of the inflamed knee joint results in chronic pain.

In certain embodiments, the bioelectromagnetic methods according to the present disclosure may be applied for treatment of injured or diseased bone for promoting the growth and repair of bone tissue in vivo. The bone micro-environment is composed of intercellular calcified material, osteoblasts, osteocytes and osteoclasts while the extracellular matrix comprises an organic component of collagens, proteoglycans, hyaluronan and other proteins, phospholipids and growth factors. The mineralized inorganic component is predominantly crystallized calcium and phosphorus in the form of hydroxyapatite. The method described herein has an effect in vivo to release BMP-2, BMP-7, for proliferation and differentiation of osteoblasts to increase the number of osteoblasts for mineralization, to enhance the mineralization step and ossification of new bone tissue, to modulate the activity of calcium/calmodulin-mediated actions as well as G protein coupled receptors and mechanoreceptors, and increase bone density. This enhances the generation of sufficient tissue for proper tissue healing in vivo.

The bioelectromagnetic therapy described herein is suitable for accelerating healing of bone fractures including, but not limited to accidental occurrences or deliberate surgical intervention, to promote fusion of vertebrae after spinal fusion surgery and treat osteopenia and osteonecrosis.

More specifically, bone fractures are categorized as simple and compound fractures and further subdivided as: simple fracture (closed fractures) that occur when a bone suffers breakage but does not pierce through the epidermis; compound fracture, opposite to simple fracture and is also known as an open fracture involving luxation of the bone that pierces through the epidermis and thus susceptible to infection; oblique fracture where the fissure runs diagonal to the axis of the bone; transverse fracture that is perpendicular to the axis of the bone; spiral fracture involving a fracture line that twists around the bone; comminuted fracture where the bone will be broken into several fragments; liner Fracture where the break is parallel to the long axis of the bone; greenstick fracture, partial fracture with one side of the bone unharmed; impacted fracture where the bone splits into two fragments; Complete and Incomplete Fractures; Compression Fracture where at least two bones are forced against one another; Avulsion Fracture, a closed fracture that occurs when the bone breaks due to a forceful contraction of a muscle; Stress Fracture (hairline fracture) due to overuse; Displaced Fracture where the bone breaks into two parts in a way that the bone loses its alignment; Non-Displaced Fracture where the bone snaps into two pieces but stays aligned; Fatigue Fracture where the bone becomes traumatized because of mundane stressors which cause weakness over a period of time; and Pathological Fracture as a result of an underlying health condition, such as osteoporosis or if cancer cells spread to the bones.

In aspects, the method has use to accelerate the healing of damaged or torn cartilage associated with injured bone

In aspects, the method has use for the treatment of bone diseases such as but not limited to osteoporosis, metabolic bone disease, bone cancer, and scoliosis.

In aspects, the method has use for the treatment of joint diseases such as but not limited to osteoarthritis, rheumatoid arthritis, spondyloarthritis, juvenile idiopathic arthritis, lupus, gout, and bursitis.

In certain embodiments, the bioelectromagnetic methods according to the present disclosure may be applied for treatment of cancer in conjunction with surgery, radiation therapy and chemotherapy. Examples of cancer include, but are not limited to, breast cancer, skin cancer, bone cancer, prostate cancer, liver cancer, lung cancer, brain tumor (glioma), head and neck cancers, colon cancer, osteosarcoma, small cell lung tumor, smooth muscle tumor, osteosarcoma and other sarcomas.

In the embodiment of a brain tumor (e.g., glioma) the personalized bioelectromagnetic method herein described may help reduce uncontrolled cell division and, after cancer tumor eradication, help to regenerate tissue/organs to health and function which includes for example stem cell homing, controlled proliferation, differentiation and blood vessel sprouting, growth and maturation expression proteins.

In certain embodiments, the personalized bioelectromagnetic methods according to the present disclosure may be applied for treatment of a pain-related disorder and the therapeutic response includes a reduction or elimination of pain experienced by the patient. Examples of pain-related disorders include, for example, pain response elicited during tissue injury (e.g., inflammation, infection, and ischemia), and pain associated with musculoskeletal disorders (e.g., joint pain such as that associated with arthritis, toothache, and headaches).

In some implementations, the personalized bioelectromagnetic methods according to the present disclosure may be applied for the reduction or elimination of pain associated with an injury or disease and may include but not be limited to adhesive capsulitis, tennis elbow, osteoarthritis, back pain, multiple sclerosis, tendon inflammation, and carpal tunnel syndrome.

In some implementations, the personalized bioelectromagnetic methods according to the present disclosure may be applied for the treatment of a patient with a bone, joint, soft-tissue, or connective tissue disorder and the method reduces or eliminates inflammation in a bone, joint, soft-tissue, or connective tissue of the patient and thus leads to a reduction or elimination of pain associated with the disorder.

In some implementations, the personalized bioelectromagnetic methods according to the present disclosure may be applied for the treatment of a dental condition, and the therapeutic response includes a reduction or elimination of pain associated with the dental condition.

In some implementations, the personalized bioelectromagnetic methods according to the present disclosure may be applied for the treatment of a patient with post-traumatic and post-operative pain and edema in soft tissues, wound healing, burn treatment, and nerve regeneration. In aspects this is by reducing inflammatory responses associated with the painful conditions. The personalized bioelectromagnetic therapy described herein may enhance the production of nitric oxide via modulation of Calcium (“Ca²⁺”) binding to calmodulin (“CaM”). This in turn can inhibit inflammatory leukotrienes that reduce the inflammatory process.

### Emf Device Configurations

An electromagnetic field device is used to provide the personalized self-adaptive bioresponsive bioelectromagnetic therapy as herein described and comprises components for generating a personalized microenvironment stimulation target and executing a personalized treatment protocol for a patient. The device can be configured in a variety of manners depending on the injury or disease and as prescribed by a healthcare practitioner for a patient. In any configuration, the device is programmable to execute the personalized microenvironment stimulation target and transmit the personalized treatment protocol for a patient. One or more processor(s)/control module(s), integral and/or external to the device, are configured to receive the operating signals for the device via the healthcare provider's electronic computing device (e.g., smartphone, laptop, tablet, etc.). In one non-limiting example a Bluetooth chip can be provided in the device and in a transceiver unit of the wearable device, and is thus able to transmit treatment and sensor data from the device to the electronic computing device, and/or operating commands from the electronic computing device to the EMF device. Data can be wirelessly transmitted from the computer implemented platform as described herein to a cloud storage, and vice versa. Some or all of the components of a therapeutic electromagnetic field delivery device may be integrated into a control circuit chip to miniaturize the device for various deployment configurations. Timing circuitry can be provided in the device or remote microcontroller, the timing circuitry configured to automatically repeat delivery of the electromagnetic waves and periods of off-time.

In an embodiment, conductive contact of the device with the anatomical area is not required to induce the electrical current in the tissue. As a non-invasive device a patient may be more psychologically prepared to experience and comply with a method incorporating its use resulting in a better outcome. Further, non-invasive methods may avoid possible negative effects to biological tissues, are generally painless and may be performed without any of the risks involved with surgery and without the need for local anesthesia. Less training may be required for use of non-invasive procedures by medical professionals utilizing the device described herein and thus suitable for use by the patient or family members at home or by first responders at home or at a workplace.

In a further embodiment, the device is configured to securably place the conductive coils directly adjacent the area targeted for treatment.

Still in further embodiments, for some applications the device or components thereof can be configured for implantation in the patient.

The device can be stationary (i.e., fixed), portable, disposable, and/or implantable. The device can be configured as a stand-alone device of any size to be used for example at home, at a clinic, hospital, treatment center and/or outdoors. The device may be suitable for prolonged or intermittent use. In some implementations the device may be placed directly over/juxtaposed with/substantially adjacent an anatomical area of a patient to provide bioelectromagnetic therapy to the injury or disease microenvironment located at that area.

A stationary configuration may include for example, but not be limited to, incorporation with furniture (e.g., bed) with respect to a mattress providing full body bioelectromagnetic treatment of a patient during periods of rest and/or sleep. The mattress may include a plurality of interconnected current carrying coils arranged in a desired pattern and operationally connected to the EMF source. Other configurations may include integration with a mattress pad, cushion, sheet, pillow, blanket, wheelchair, chair, body support for a car, exercise device and with other therapeutic and health maintenance devices as understood by one of skill in the art.

Alternatively, the device may be configured as a wearable device providing ergonomic fit to a specific anatomical area of the patient's body (e.g., head, neck, chest, shoulders, knee, foot, ankle, back, wrist, and elbow) that has an injury or disease for application of the personalized treatment protocol to a target microenvironment. A wearable device may be unisex, configured to be of any shape and size to fit any patient, lightweight, hands-free (once positioned on the patient's body) and portable due to incorporating, attaching or embedding a bioelectromagnetic EMF circuit comprising in aspects: a rechargeable and replaceable battery (alternatively, wireless operation configuration); a central processing unit; a wireless transceiver; optional display for input or for monitoring status; a power switch; one or more sensors and one or more coils. As a wearable device, the components may be miniaturized as required.

A wearable device may include but not be limited to an anatomical wrap, anatomical support, apparel, chest support (e.g., bra), hat/cap/helmet, foot ware (e.g., sneakers, boots), fashion accessory (e.g., bracelet), dressing, bandage, compression bandage and compression dressing. In an embodiment of an anatomical wrap device, such a device is shaped for encircling the particular area of the patient's body requiring treatment, such as for example an arm, leg, head, neck or hand.

A wearable wrap device comprises a means for fastening securely to the anatomical site, e.g., treatment target of the patient's body, with for example reversible fasteners such as Velcro™-like straps, hooks, snaps, combinations thereof and the like.

A wearable device may be manufactured to comprise a variety of materials that may be soft, flexible, provide stretch, body-compatible, natural or synthetic, for example, cotton, wool, polyester, rayon, Gore-Tex®, rubber, neoprene, resin or other fibers or materials known to a person skilled in the art as non-irritating and in aspects breathable (i.e., for a garment). The material may be a smart material that can sense the environment and respond to changes in strain, temperature, moisture, and pH. The material may be selected depending on the treatment target area, for example a snug fit may be desired for a wrap placed around the patient treatment target. Configurations of a wearable device may provide some structural support and may also function as an orthopedic support brace. A wearable device can be layered. A flexible plastic that can be molded about a body part is also suitable for use. Alternatively, the device can be configured within a non-flexible material such as a plaster cast. The wrap device may also include other semi-stiff components such as, for example, bendable plastic found in orthopedic applications.

A wearable device as herein described is prescribed for a patient in a personalized configuration such that once in place on the patient juxtaposed at the treatment target, the coils or electrodes are strategically positioned to effectively provide the personalized treatment protocol.

It is understood by one of skill in the art that a wearable device as herein described can be provided as a kit comprising: a bioelectromagnetic EMF circuit comprising in aspects, a rechargeable and replaceable battery (alternatively, wireless operation configuration), a central processing unit, a wireless transceiver, optional display for input or for monitoring status, a power switch, one or more sensors and one or more coils; an anatomical wrap or support, apparel, chest support (e.g., bra), hat/cap/helmet, foot ware (e.g., insoles for a pair of sneakers, boots), fashion accessory (e.g., bracelet), dressing, bandage, compression bandage and compression dressing; and instructions for use.

### Treatment Systems

The inventions described herein may be implemented as a system, it is understood that such systems may include and/or involve a variety of general-purpose computer components such as but not limited to software modules, general-purpose central processing unit (CPU) and main memory (RAM).

The inventions described herein may be implemented with disparate or different software, hardware and/or firmware components, beyond that set forth above, for example, with general purpose or special purpose computing systems or configurations not limited to: software or other components within or embodied on personal computers, servers or server computing devices such as routing/connectivity components, hand-held or laptop devices, multiprocessor systems, microprocessor-based systems, set top boxes, consumer electronic devices, network PCs, other existing computer platforms, distributed computing environments that include one or more of the above systems or devices.

The inventions described herein may in some instances be achieved via or performed by for example logic and/or logic instructions including program modules, executed in association with such components or circuitry. In general, program modules may include routines, programs, objects, components, data structures, etc. that performs particular tasks or implement particular instructions herein. The inventions may also be practiced in the context of distributed software, computer, or circuit settings where circuitry is connected via communication buses, circuitry or links. In distributed settings, control/instructions may occur from both local and remote computer storage media including memory storage devices.

Innovative software, circuitry and components herein may also include and/or utilize one or more type of computer readable media resident on, associable with, or can be accessed by such circuits and/or computing components, for example, computer readable media may comprise computer storage media and communication media. Computer storage media includes volatile and non-volatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical storage, magnetic tape, magnetic disk storage or other magnetic storage devices, or any other medium which can be used to store the desired information and can accessed by computing component. Communication media may comprise computer readable instructions, data structures, program modules and/or other components. Communication media may include wired media but not transitory media.

Aspects of the method, device and system as described herein with respect to the logic, can be functionality programmed into any of a variety of circuitry, including programmable logic devices (“PLDs”), such as field programmable gate arrays (“FPGAs”), programmable array logic (“PAL”) devices, electrically programmable logic and memory devices and standard cell-based devices, as well as application specific integrated circuits. Other aspects of the method, device and system as described herein can implement memory devices, microcontrollers with memory (such as EEPROM), embedded microprocessors, firmware, software and the like. Aspects may be embodied in microprocessors having software-based circuit emulation, discrete logic (sequential and combinatorial), custom devices, fuzzy (neural) logic, quantum devices, and hybrids of any of the above device types.

Logic and/or functions disclosed herein may be enabled using any number of combinations of hardware, firmware, and/or as data embodied in various machine-readable or computer-readable media, in terms of their behavioral, register transfer, logic component, and/or other characteristics.

The need for the present invention will be further illustrated in the following examples. However, it is to be understood that these examples are for illustrative purposes only and should not be used to limit the scope of the present invention in any manner.

### Experimental Examples Demonstrating Proof of Concept

These examples are provided for purposes of illustration only and are not intended to be limiting unless otherwise specified. Thus, the invention should in no way be construed as being limited to the following examples, but rather, should be construed to encompass any and all variations which become evident as a result of the teaching provided herein.

To support the assertion that electromagnetic therapy should be personalized to a specific patient and the biology of the injury/disease to be treated, the effects of EMF stimulation on biological effects at the cellular level between different donors was examined (Examples 1-3).

The results demonstrated that the baseline level of biological activity at the cellular level for different donors was fundamentally different, as would be expected. Furthermore, when the same EMF exposure was delivered to cells from different donors (inter-donor variability) the biological effects at the cellular level were different. Also, when cells from the same donor were exposed to different electromagnetic fields (intra-donor variability), the resulting biological effects were different. Inter- and intra-patient differences support the need for personalization and optimization of treatment for a patient.

EMF stimulation profiles demonstrated differential gene expression in donor mesenchymal stem cells (Example 1). An identical EMF exposure was demonstrated to differentially up-regulate or down-regulate genes for each of five donors. As quantified by gene expression, each donor cell group had a different response. In addition, one donor was subjected to different stimulation profiles but only one (1 mT, 75 Hz pulse administered for 10 minutes per day) produced marked increases in osteogenic and chondrogenic gene expression. This same exposure generated different responses in each of the donors.

EMF exposure profiles also demonstrated intra-donor variability in the breast cancer cell line MDA-MB-231 with respect to cell growth and gene expression involving genes belonging to apoptosis, cellular senescence and angiogenesis pathways (Example 2). EMF was demonstrated to enhance the effect of the cisplatin (a chemotherapeutic agent) supporting the use of the personalized bioelectromagnetic therapy described herein as an adjunct to traditional chemotherapy. EMF exposure profiles also demonstrated inter- and intra-donor variability with respect to astrocyte cell donors (Example 3) where gene expression levels along inflammation signalling pathways were altered.

The experimental results also support the use of gene expression as a useful tool to monitor and characterize the effects of EMF treatment on the cells of the injury/disease microenvironment and may further provide guidance for identifying the optimal initial treatment protocol, amending the treatment protocol during treatment, or for the use of an adjunct therapy. A baseline gene expression profile can be obtained from patient cells prior to the start of treatment to determine EMF gene targeting effects

This clinical data would be supplemental data to include in the “Patient Profile” as one of the variable biological parameters fed to MiCE to compute a personalized microenvironment stimulation target for the patient. Any appropriate cell type may be extracted/harvested from a patient from for example a blood draw, a mouth swab, or invasively from the microenvironment. Gene expression and control levels can be assessed at different time points throughout the treatment protocol to allow progressive therapy optimization. The selection of genes may be dependent on the cell/tissue type as well as on the type of injury and disease. Non-limiting examples of genes for expression array assessment are listed in Appendix I-III.

The following examples further illustrate the need for and the implementation of the present invention. However, it is to be understood that these examples are for illustrative purposes only and should not be used to limit the scope of the present invention in any manner.

### Example 1: EMF Induced Gene Expression Variability—Inter-Donor and Intra-Donor Bone Marrow-Derived Mesenchymal Stem Cells

This in vitro experimental study demonstrates variability in the response of cells from a specific donor to different EMF stimulation (intra-donor differences) and that, for equivalent EMF stimulation, there is donor-to-donor (i.e., inter-donor) differences. The response of different donor cells to various harmless and undetectable extremely-low frequency magnetic fields is compared using a PCR array. Outcomes relevant to osteogenesis and bone repair are quantified and compared using relative gene expression levels.

The data collected from the mesenchymal stem cell-specific PCR array provide evidence for variable gene expression depending on the donor and the EMF stimulation. The same exposure was shown to up-regulate or down-regulate genes differently for each donor and the response of a donor cell group was specific to the EMF field parameters. Donor PC-1 was exposed to six different stimulation profiles and observed marked increases in osteogenic and chondrogenic gene expression when subjected to a 1 mT, 75 Hz pulse for only 10 minutes per day. This same exposure did not generate a similar response from four other donors. While beneficial to PC-1, this exposure has not been optimized but demonstrates, at a cellular level, the need for donor-to-donor personalization and optimization for development of personalized treatment protocols as described herein.

The technology described herein utilizes and may ‘learn’ from the identified baseline gene expression differences inherent in the patient profile, and further influenced by the biological challenge, to compute a personalized microenvironment stimulation target and then actively optimize the treatment during the fracture's exposure to EMF.

### Materials and Methods

### Cell Donors—Mesenchymal Stem Cells

Bone marrow-derived mesenchymal stem cells (BM-MSC) were procured for a wide range of donors, each possessing a unique patient profile (Table 2). MSCs from donors with no known pre-existing medical issues (Donor LZ-1) were obtained from Lonza (Lonza Walkersville, Walkersville, MD, USA). Cells sourced from PromoCell (PromoCell GmbH, Heidelberg, Germany) offer an expanded donor profile that includes age, sex, ethnicity, smoking status, body-mass index, and whether they suffer from osteoarthritis (Table 2). MSCs from four different donors, obtained from PromoCell, provide a diverse set of characteristics (Donors PC-1 through PC-4). Comparing baseline gene expression levels between donors (inter-donor) prior to exposure reveals significant differences that logically are likely to affect response to EMF stimulation.

TABLE 2
List of mesenchymal stem cell donors procured for EMF treatment

| | | | | | | | Presumed |
|---|---|---|---|---|---|---|---|
| Donor ID | Age | Sex | Ethnicity | [symbol] | [symbol] | Smoker status | Health Status |
| LZ-1 | <45 | — | — | — | — | — | Young, |
| | | | | | | | Healthy |
| PC-1 | 68 | Male | Caucasian | 26.1 | No | Non-smoker | Older, |
| | | | | | | | Over-weight |
| PC-2 | 72 | Female | Caucasian | 37.2 | Yes | Smoker | Older, |
| | | | | | | | Unhealthy |
| PC-3 | 44 | Female | Caucasian | 27.9 | Yes | Never- | Middle-aged, |
| | | | | | | smoker | Over-weight |
| PC-4 | 92 | Female | Caucasian | 20 | Yes | Never- | Elderly, |
| | | | | | | smoker | Healthy |

[symbol]

indicates data missing or illegible when filed

### Cell Culture

Bone Marrow derived Mesenchymal stem cells (BM-MSCs) were sourced from different donors (PromoCell: C-12974/Lonza: PT-2501). Cells were thawed from frozen (−150° C.) and plated into a T75 flask containing DMEM supplemented with 1% GlutaMAX™ and 5% human platelet lysate. Cells were placed in an incubator set to 37° C. and 5.0% CO2. Media was exchanged 1 day after thawing to remove the DMSO. Media was exchanged every 2-3 days thereafter. Cells proliferated until 70-90% confluency. Upon reaching this threshold, cells were washed with DPBS and detached using TrypLE Select. The detached cells were neutralized, centrifuged, and seeded in culture vessels at a concentration of 5000 cells/cm² for passaging.

For PCR analysis, the dissociated cells (approximately 500,000 cells) were neutralized using an equal volume of complete media and centrifuged for 2 minutes at 4000 RPM. Following centrifugation, the supernatant was aspirated, and the cell pellets were immediately frozen at −80° C. for future RNA extraction.

### Electromagnetic Field Stimulation

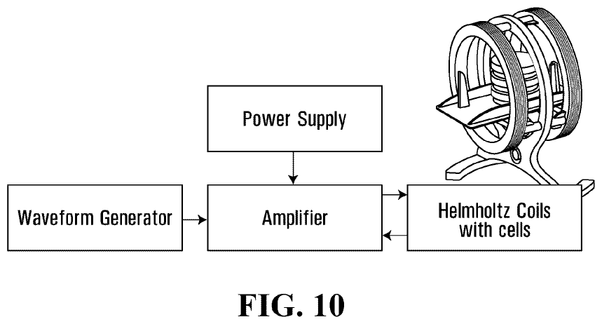

FIG. 10 schematically depicts set up for application of electromagnetic fields to cultured cells. Cells were exposed to a spatially uniform, time-varying magnetic field using Helmholtz coils which is the configuration of two copper magnet wire coils of equal number of turns and diameter that are axially aligned and separated by a distance equal to their radii. The space within the Helmholtz coils creates a large volume wherein the induced magnetic field is uniform (within 5%). Experiments were performed in pairs of internally prepared coils with diameter 10 cm, or 30 cm Helmholtz coils purchased from Serviciencia (Serviciencia, S.L.U., Spain). The useable region of uniform magnetic field can be approximated as a cylinder of diameter 4.2 cm and length 4.96 cm for the small coils and 25.4 cm by 29.3 cm long for the larger coils, accommodating chamber slides and 6-well plates, respectively. Culture dishes stacked within the uniform region all experience an identical alternating magnetic field. Several fields were investigated by changing one or more parameter from waveform, frequency, magnetic flux density and exposure timing. The Helmholtz coils are vertically oriented such that the magnetic field lines are horizontal-perpendicular to the axis of the wells on a 6-well plate. The coils and Exposed cells (those receiving EMF stimulation) were placed directly inside an incubator at 37° C. and local temperatures were closely monitored. Control cells for each donor were cultured for the same length of time, and at the same temperature and CO₂ settings, but in a separate incubator isolated from the stimulating fields.

Stimulation in vitro profiles were designed consisting of biphasic alternating current (AC) magnetic fields whose waveforms were sinusoidal or pulsed. The driving waveforms (signals) varied not only in shape, but also frequency and amplitude. Table 3 lists details of each exposure evaluated, as well as the daily stimulation time and time spent in culture. The pulsed waveforms are driven using a biphasic rectangular wave with a 10% duty cycle and each of Experiment 1-3 has different daily exposure times. Sine wave inputs (Experiments 4-6) oscillate about zero (i.e., zero DC offset) and vary only in frequency. Stimulation profiles were varied for the same donor to demonstrate intra-donor variability (PC-1). Inter-donor comparisons are made by exposing cells from each of the five donors to the same electromagnetic field therapy (Experiments 1 and 5 for pulsed and sinusoidal stimulation, respectively).

TABLE 3
Waveform parameters and exposure timing for each experiment

| | | | | | Daily | Exposure |
|---|---|---|---|---|---|---|
| Experi- | | | B | f | exposure | length |
| ment | Donors | Waveform | (mT) | (Hz) | (minutes) | (days) |
| 1 | ALL | Pulse | 4 | 75 | 10 | 4-7 |
| 2 | PC-1 only | Pulse | 4 | 75 | 240 | 5 |
| 3 | PC-1 only | Pulse | 4 | 75 | Continuous | 5 |
| 4 | PC-1 only | Sine | 1 | 15 | 240 | 4 |
| 5 | ALL | Sine | 1 | 50 | 240 | 4-7 |
| 6 | PC-1 only | Sine | 1 | 250 | 720 | 4 |

-

Current through the coils was generated by a DG2052 waveform generator (Rigol Technologies, China) and then amplified by a BOP 100-4DL power supply (KEPCO, INC., USA). The waveform generator can generate sine waves up to 50 MHz (square wave at 15 MHz), however, stimulation frequencies were kept to the extremely low frequency range (<300 Hz). Low intensity, AC magnetic fields in this frequency range produce no heat or sound and have been shown to be harmless. The magnetic flux density of the induced field is at any instance directly proportional to the current in the coils (monitored using RP1001C current probe (Rigol Technologies, China)) and was measured using a 5180 gaussmeter and SAD18-1904 axial probe (F.W. Bell, USA). The maximum peak magnetic flux density was 4 mT. At high current, dissipated power in the coils resulted in elevated coil temperatures, however, the heating effect was negligible at the centre of the coils where the cells are situated. Exposure timing was controlled via remote commands sent to the waveform generator via LabVIEW (National Instruments, USA).

### Imaging

Cells were imaged prior to each feed and on the day of termination using a Zeiss Axio Vert A1 Inverted Microscope at 50× magnification.

### PCR Array

RNA was extracted from frozen cell pellets using a RNeasy kit (Qiagen: 74004) and subsequent cDNA was reverse transcribed (Qiagen: 330404). Reverse transcribed templates ware analyzed by a Mesenchymal Stem Cell PCR Array (MSC RT² Profiler Array (Qiagen: 330231; PAHS-082ZD)) on a BioRad CFX96 Real Time PCR Detection System. Each experimental point was performed in triplicate. Gene expression of the 84 genes of interest on each PCR array was normalized to reference genes (ΔCₜ) and then averaged within an experimental group. ΔΔCₜ for each gene was then calculated between groups before fold change was calculated according to 2^{(-ΔΔCt)}. In the results below, some data is presented in the form of heatmaps, where the fold change is displayed at log base 2.

### EMF Exposure Does Not Affect Cell Morphology

Control and Exposed donor cell culture groups were imaged before each feed and before termination to monitor morphology. There were no morphological changes observed with or without EMF stimulation of any intensity or duration.

### PCR Array Analysis Reveals Inter-Donor Variability In Absence Of Electromagnetic Field

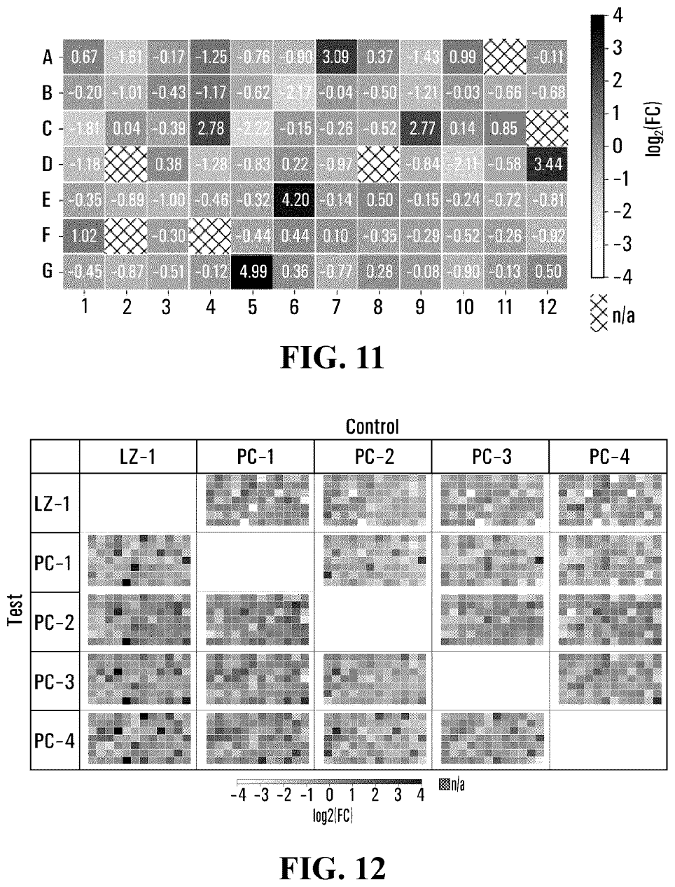

A PCR array designed specifically for mesenchymal stem cells (Qiagen) measures the expression levels of 84 different genes, A1 to G12, listed in Appendix I. Genes targeted in the array include stemness markers, differentiation markers, and other known mesenchymal stem cell specific genes. RNA isolated from Control cells belonging to each donor were analyzed using the PCR array to elucidate unstimulated baseline expression differences between donors (note that Controls are also used to quantify the effect of EMF exposure). FIG. 11 shows a PCR array heatmap for the relative difference between the expression levels of PC-1 and LZ-1 (where LZ-1 is the chosen “calibrator”). The colour-bar corresponds to log-base 2 of the fold change (FC) between the two donors such that red (positive values) corresponds to up-regulation of a gene and blue (negative values) a down-regulation. The example in FIG. 11 shows five genes (A7: BGLAP, C4: GDF6, C9: HGF, E6: NGFR, and G5: THY1) that are strongly up-regulated by PC-1 compared to LZ-1.

FIG. 12 is an array of gene expression heatmaps comparing each donor's baseline expression level to that of the other donors. When cultured without any form of external stimulation, qualitatively, there are distinct differences between the gene expression of each donor. The differences observed in FIG. 12 cell groups as well as the Controls response to EMF treatment may be used as a basis for developing personalized treatment specificity.

### Inter-Donor Variability In Response To EMF Exposure

### Pulse Wave Stimulation—Experiment 1

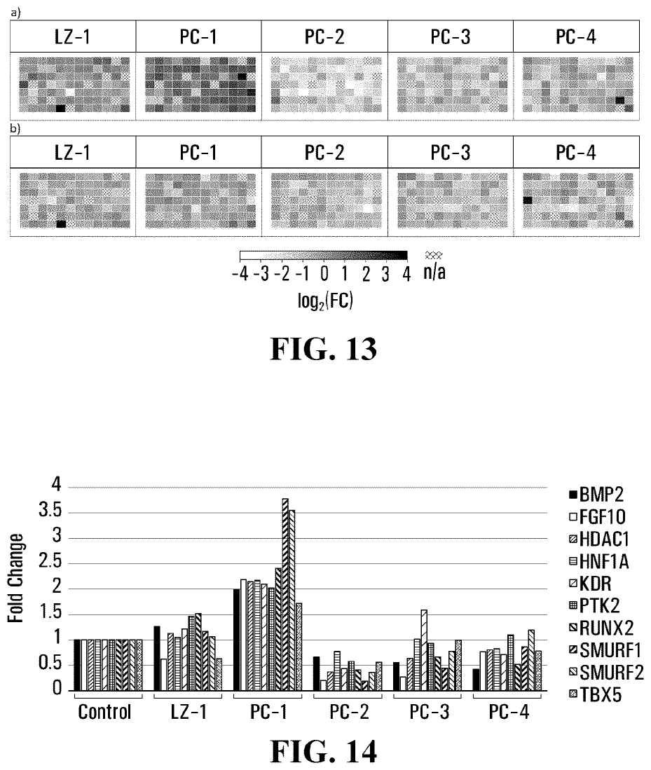

All five donor cell groups were exposed to a pulsed magnetic field following cell seeding. The field parameters were set according to Experiment 1 in Table 2 where the input pulse width was 1.3 ms (duty cycle=10%). Cells were exposed for 10 minutes per day until confluent. Cells were not exposed on the day the cells were terminated. FIG. 13 compares the gene expression of each set of donor cells, relative to its own unexposed control, when exposed to the pulsed field. FIG. 13(a) alternating magnetic fields 75 Hz, 4 mT pulse waveform; and FIG. 13(b) alternating magnetic fields 50 Hz, 1 mT sine wave. On average, gene expression is slightly up-regulated in Donor LZ-1 relative to the others, but the effect is much stronger for PC-1 where most of the genes are up-regulated. Expression in PC-2, PC-3, and PC-4, by comparison, are all down-regulated-PC-2 strongly so. These MSCs exhibit inter-donor specificity to the stimulation profile.

By selecting specific osteogenic genes from the PCR arrays shown in FIG. 13, it is possible to focus on and emphasize the differences in the heatmaps in terms of specific responses attributed to a target biological process. FIG. 14 plots the expression levels of ten osteogenic genes as part of Experiment 1. The pathways associated with each of these ten genes promote osteogenesis and, hence, processes associated with osteogenesis would benefit from up-regulated gene expression. There is significant up-regulation in PC-1 compared to the other donors: expression levels have a fold change of at least 2 for all ten genes in the PC-1 test, whereas PC-2, PC-3, and PC-4 have little to no up-regulation with the same exposure protocol. Some of the highly-expressed genes in PC-1 that are crucial for fracture repair include: Bone morphogenic protein-2 (BMP2) is a growth factor of the transforming growth factor-beta (TGF-β) superfamily that plays an essential role in osteogenesis and takes part in inducing cartilage and bone formation; RUNX2 is heavily expressed in bone marrow and activates the differentiation of MSCs into immature osteoblasts; TBX5 promotes bone growth and maturation; and FGF10 and SMURF1 are both involved at different points in the BMP pathway.

### Sinusoidal Stimulation—Experiment 5

Exposing cells from the five donors to an oscillating, sinusoidal magnetic field (Experiment 5 in Table 2) yields markedly different results. The sine wave (apart from being lower intensity and frequency than Experiment 1 has more gradual gradients than a pulse wave and will result in weaker induced electric currents. Figure C(b) shows gene expression heatmaps post-exposure to a sine wave for 6 hours a day. The sine wave produced much less extremes than the pulsed signal and there are fewer distinguishing factors from donor to donor. This result (particularly the lack of a significant response from PC-1) suggests that some EMF treatments influence biological responses at the cellular level in different ways on a patient-by-patient basis.

### Intra-Donor Variability Demonstrated By Varying EMF Treatment Parameters

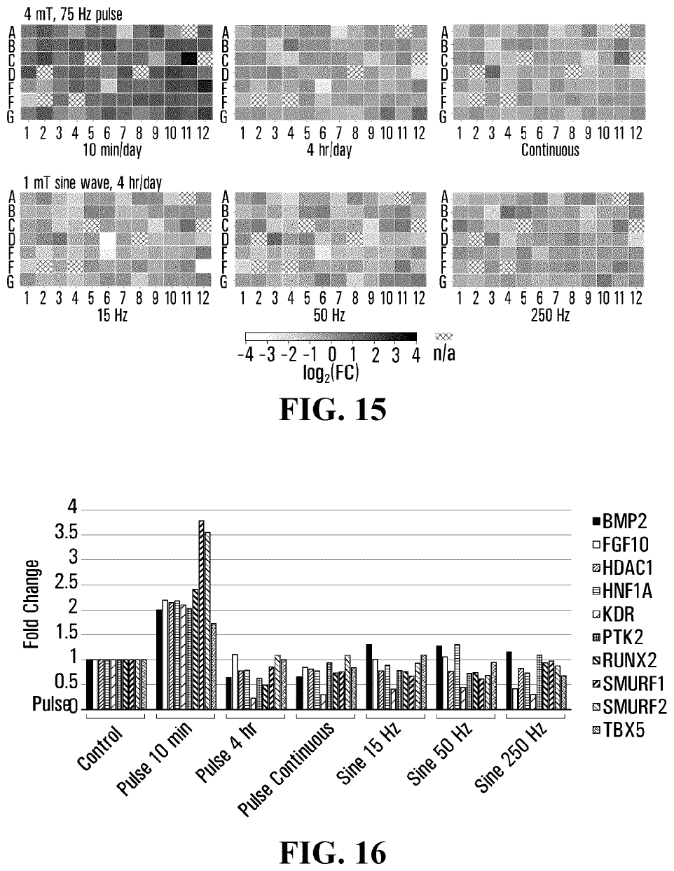

The field parameters defined in Experiments 1 to 6 (Table 3) were each used to stimulate PC-1 during growth. After normalizing to an unexposed control, the heatmaps in FIG. 15 reveal the intra-donor variability when exposing cells to different waveforms at different intensities, frequencies, and exposure times. Gene expression is most up-regulated by short daily exposures to pulses. Longer exposure results in more strongly down-regulated genes, suggesting that optimization is possible via exposure timing. Additionally, changing to a sine wave produced substantially less change from controls than each of the pulsed waveforms. The magnitude of the fold change values (either positive or negative) is greatest at low frequency for the sinusoidal magnetic field exposures. These results imply that EMF treatment can be fine-tuned at the cellular level to a given donor, thereby generating a personalized therapeutic regime specifically optimized for a microenvironment.

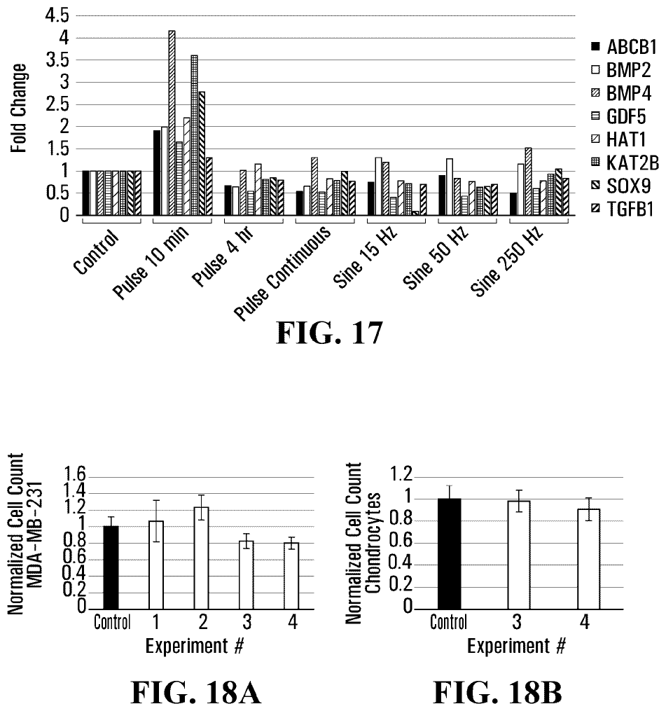

The different responses to EMF treatments are highlighted by selecting and focusing on ten osteogenic genes (FIG. 16) and ten chondrogenic genes (FIG. 17). A 10-minute daily exposure to the 75 Hz, 4 mT pulse strongly up-regulates genes related to osteogenesis and chondrogenesis. The continuous exposure has some up-regulated genes but, similarly, others that are strongly down-regulated. Each of the genes up-regulated by the 10-minute exposure, including BMP2, RUNX2, etc. (described in detail above), are involved in signaling pathways that promote osteogenesis and chondrogenesis.

### Example 2: EMF Therapy Inhibits Cell Growth of MDA-MB-231 Breast Cancer Cells and Enhances the Effects of Chemotherapy Drug, Cisplatin

A breast cancer cell line (MDA-MB-231) was cultured in the presence of various time-varying electromagnetic fields capable of inducing electric currents in the culture media and across the cells themselves. Proliferation cultures with four different exposure profiles yielded a different response from each experiment. Two exposures caused a statistically significant decrease in cell growth (a positive outcome for inhibiting cancer cells). The two exposure profiles were (i) a 432 Hz sine wave administered continuous from seeding to termination and (ii) a series of low frequency triangular waves with increasing separation for 3 hours per day. These two waveforms were investigated further using PCR arrays configured for cancer-related pathways. Despite the two exposures having a very similar effect on cell count, intra-donor variability was observed via the PCR arrays. Genes belonging to apoptosis, cellular senescence and angiogenesis pathways, to name a few, were all up-regulated in favour of inhibited cell growth. However, some indicators of increased proliferation and apoptosis inhibitors were also up-regulated. Additionally, the benefit of bioelectromagnetic therapy as an adjunct to chemotherapy was demonstrated using cisplatin. When the two modalities are combined, the EMF acts to enhance the effect of the cisplatin and cell count are significantly decreased.

These results support personalized treatment optimization using EMF based on the type of cancer for slowing tumour growth demonstrating bioelectromagnetic therapy as an attractive alternative (or adjunct) to traditional chemotherapy and/or radiotherapy.

### Materials and Methods

### Cell Culture

MDA-MB-231 cells, a human triple-negative breast cancer cell line, were obtained at passage 40 (Sigma: 92020424). The culture media used was high glucose DMEM (Gibco: 31053-028) supplemented with 1% GlutaMAX™ (Gibco: 35050-061) and 10% fetal bovine serum (Gibco).

Cells were thawed from frozen (−150° C.), placed in a tube with warm media and spun down for 5 minutes at 240×g. After pelleting the cells, the media was removed, and the cells resuspended and seeded into a T225 flask with 45 mL of media. Cells were placed in an incubator set to 37° C. and 5.0% CO₂. Media was exchanged every 2-3 days. Cells proliferated until they reached 80-90% confluency. Upon reaching this threshold. cells were passaged. Cells were washed with DPBS and detached using TrypLE Express (Gibco). The detached cells were neutralized, centrifuged, and seeded in culture vessels at a density of 10,000 cells/cm².

For experimental harvests cells were detached as when passaging and neutralized with media. A 100 μL aliquot was taken and used for cell counts. Cells were counted on a NC-200 Nucleocounter (ChemoMetec). The remaining dissociated cells were centrifuged for 2 minutes at 4000 RPM. Following centrifugation, the supernatant was aspirated, and the cell pellets were immediately frozen at −80° C. for future RNA extraction.

Cells were imaged prior to each feed and on the day of termination using a Zeiss Axio Vert A1 Inverted Microscope at 50× magnification.

### Cisplatin Preparation

Cisplatin is a platinum-based chemotherapy drug that inhibits DNA synthesis. Cisplatin is used in the treatment of many cancers, including breast cancer. Cisplatin (Millipore-Sigma: 232120) was dissolved in DPBS with 140 mM NaCl at a concentration of 1 mg/mL and stored at room temperature, protected from light. The solution was further diluted in culture media to obtain the desired concentration for experiments. DPBS with 140 mM NaCl was used as a vehicle control. Cisplatin at 20 μM was used as a positive control as this concentration reduced the cell number by more than 90%. Lower concentrations of 2 μM and 0.667 μM (one tenth and one thirtieth the concentration of the positive control, respectively) were used in combination with PEMF stimulation.

### PCR Array

RNA was extracted from frozen cell pellets using a RNeasy kit (Qiagen: 74004) and subsequent cDNA was reverse transcribed (Qiagen: 330404). Reverse transcribed templates ware analyzed by a Human Cancer PathwayFinder™ PCR Array (Human Cancer PathwayFinder™ RT² Profiler Array (Qiagen: 330231; PAHS-033Z)) on a BioRad CFX96 Real Time PCR Detection System. Each experimental point was performed in triplicate. Gene expression of the 84 genes of interest (Appendix II) on each PCR array was normalized to reference genes (ΔCₜ) and then averaged within an experimental group. ΔΔCₜ for each gene was then calculated between groups before fold change was calculated according to 2^{(-ΔΔCt)}. In the results below, some data is presented in the form of heatmaps, where the fold change is displayed at log base 2.

### Electromagnetic Field Stimulation

Cells were exposed, as previously described for mesenchymal stem cells, to a spatially uniform, time-varying magnetic field using Helmholtz coils. A description of the exposure system (coils, signal generator, incubators, etc.) is described in Example One.

Demonstrated are effects of four low-frequency magnetic fields (are non-invasive and cause no pain) on the growth and gene expression on a breast cancer cell line. Extremely low frequency magnetic fields on cancerous cells have demonstrated inhibition of proliferation (Bergandi L, Lucia U, et al., BBA—Mol Cell Res. 2019; 1866:1389-1397) and/or increased apoptosis (Giladi M, Schneiderman R S, et al., Sci Rep. 2015; 5:18046). Table 3 contains the parameters describing four different fields covering a wide range of shapes, intensities and frequencies. The two sinusoids are simple sine waves with no offset. The pulse waveform has a 10% duty cycle, creating sudden, large changes in the field's magnetic flux density (B). Finally, Experiment 4 consists of pairs of triangular waves with increasing separation equivalent to a decreasing frequency from 36 Hz to 10 Hz. The waveform consists of 15 pairs and has a period of approximately 900 ms. Positive results with Experiments 3 and 4 (described below) ledto repeating the exposures with the addition of a chemotherapy drug cisplatin). Additionally, Experiments 3 and 4 were also performed with “healthy” chondrocytes.

TABLE 4
Experiments using different EMF stimulation profiles

| | | | | Daily | Exposure |
|---|---|---|---|---|---|
| | | Bₚₑₐₖ | f | exposure | length |
| Experiment | Waveform | (mT) | (Hz) | (minutes) | (days) |
| 1 | Sinusoid | 1.6 | 8 | 720 | 4 |
| 2 | Pulse | 2.5 | 70 | 180 | 4 |
| 3 | Sinusoid | 0.5 | 432 | Continuous | 5 |
| 4 | Triangular pairs | 0.2 | 10-36 | 60 | 5 |

### EMF Stimulation Inhibits Growth Of MDA-MB-231 Cell Line

Breast cancer cells were exposed to one of the four experimental stimulation profiles described in Table 4 during the entirety of the culture (4-5 days). The desired outcome is decreased proliferation to inhibit the uncontrolled division of cancer cells. FIG. 18(A) shows the normalized cell counts using each of the stimulation profiles compared to an unexposed control. When the breast cancer cells were treated with the exposures in Experiment 1 and 2 there was no effect and increased proliferation, respectively. However, when the same cell line is exposed to Experiment 3 or 4, the effect is reversed and cell growth is inhibited (statistically significant, Student's t-test, p<0.01). To ensure EMF exposure was not harmful to healthy, non-cancerous cells that will inevitably be subjected to the same magnetic field, Experiments 3 and 4 were repeated with human chondrocytes. The normalized cell counts in FIG. 18(B) show there is no statistically significant differences between the growth of chondrocytes with or without the same exposures that inhibit breast cancer cell growth.

### Exposure To A Time-Varying EMF Alters The Gene Expression Of Breast Cancer Cells

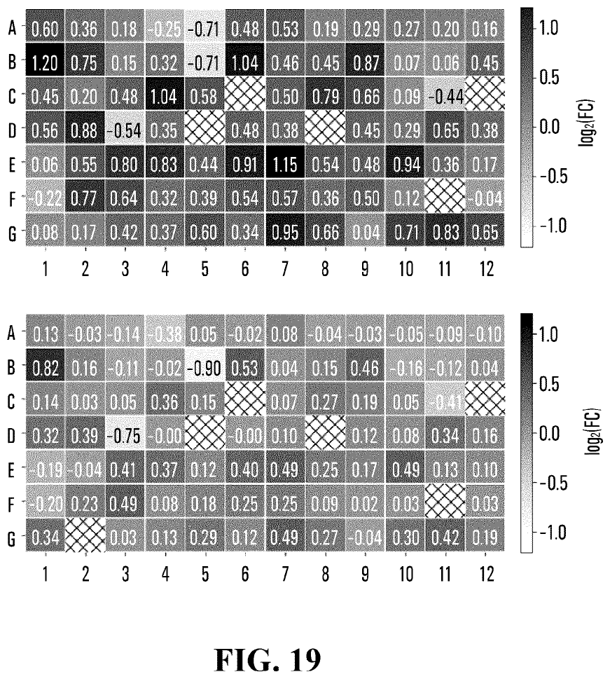

The effective stimulation profiles (3 and 4) were analyzed further using a cancer pathway finder PCR array designed specifically for cancer cells. The array contains primers for 84 genes related to, for example, one or more of apoptosis, cellular senescence, and angiogenesis. A list of the genes is included in Appendix II. The heatmaps in FIG. 19 show the relative differences between the gene expression of the two exposed samples and the control cells. The 432 Hz sinusoid in Experiment 3 appears to have a stronger effect than the triangular pulses and more genes are up-regulated relative to the unexposed control. However, the genes that are most strongly expressed (both up-regulated and down-regulated) are common to both exposures.

The biological mechanism of cancer cell growth inhibition with EMF exposure is poorly understood and on-going research continues to investigate the effects of an incredibly wide range of EMF parameters and delivery methods, within the extremely low frequency range and beyond. The gene expression levels for the two seemingly effective stimulations in Figure I highlight genes involved in affected pathways. The decreased cell counts post exposure can be related to increases in the expression of pro-apoptotic genes APAF1 (heatmap position: A6) and CASP2 (B2), however, apoptosis inhibitors NOL3 (E8) and XIAP (G12) are also up-regulated. Further, cellular senescence is generally promoted causing cell proliferation arrest: IGFBP3 (D7) and IGFBP7 (D9), MAP2K3 (E4) and MAPK14 (E5) all have increased expression. Markers of DNA damage and repair (DDB2 (C1), PPP1R15A (F2) and GADD45G (D3)) changed in favour of apoptosis and tumour suppression. To the contrary, many of the genes related to cell cycle pathways (e.g., MCM2 (E6) and MKI67 (E7)) are typically high in cancer cells and have been further expressed post-exposure, which would be in favour of increased proliferation. Finally, ANGPT1 (A4) and CCL2 (B5), both genes involved in angiogenesis that recruit blood supply for growing tumours, are down-regulated by the EMF. The genes discussed are some of the more strongly regulated genes by either stimulation.

### EMF Exposure Enhances The Effects Of Cisplatin

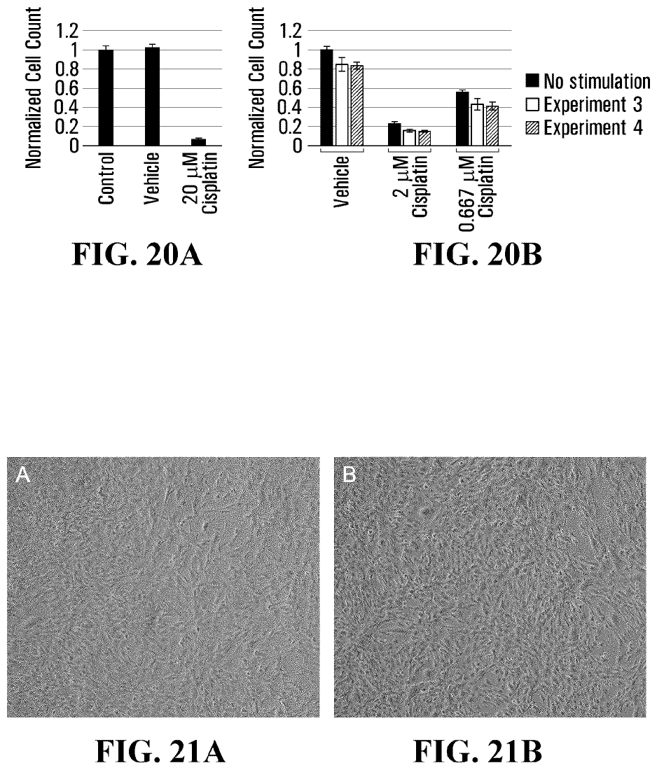

Chemotherapy drugs, including cisplatin, are a commonplace and proven method of treating cancer tumours despite a plethora of side effects. Large doses of these drugs can be a significant monetary and health concern for many patients so any method of reducing the dosage with an adjunct therapy is welcome. To test the combined effect of cisplatin and EMF exposure, two low concentrations (2 μM and 0.667 μM) of cisplatin were added to the culture media of MDA-MB-231 cells subjected to Experiment 3 and 4 exposures. Additionally, cells were cultured with 20 μM of cisplatin and no exposure as a “positive” control. FIG. 20 shows the combinatory effect of cisplatin and EMF exposure on cell count after 5 days of exposure. Without stimulation, the vehicle media is seen to have no effect on proliferation relative to control, whereas a large dose of cisplatin (20 μM) has a drastic impact on the cell count. The exposed cell groups are plotted relative to the unstimulated vehicle sample and when cultured with the vehicle show the same decrease in cell count as in Figure H (without vehicle). The cellular response to cisplatin is apparent and there is a non-linear negative relationship between concentration and cell count. When EMF exposure is added at the two lower cisplatin concentrations the cells are further inhibited. The difference between low concentrations of cisplatin alone and cisplatin with either EMF exposure is statistically significant. The effect of cisplatin and EMF exposure do not appear to be additive but instead the EMF stimulation enhances the inhibition of cell growth induced by the cisplatin. At 10% of the positive control concentration of cisplatin, the combined treatment reduces cell counts by more than 80% (relative to negative control) compared to the 90% reduction seen with the 20 μM cisplatin dose.

### Example 3: Bioelectromagnetic Therapy Alters the Reactive State of Astrocytes In Vitro

The effect of EMF stimulation on pain-related gene expression in normal human astrocytes sourced from three different donors was tested. The findings demonstrate inter-donor and intra-donor variability to different EMF stimulation profiles. This supports that patient-to-patient differences will affect their response to EMF treatment at a cellular level and that a personalized parameter set will be required for each patient to achieve a desired outcome.

Astrocytes are an abundant cell type in the central nervous system and are included in many essential processes required to maintain a healthy system. Astrocytes were chosen for this in vitro study because they are involved along pain perception and modulation pathways where they are responsible for producing and regulating pro-inflammatory and anti-inflammatory substances. In the case of chronic pain, reactive astrocytes may accentuate pain perception and inflammatory responses long after the pain-inducing injury occurred. It is presently demonstrated that the expression of inflammatory genes in reactive astrocytes can be down-regulated using EMF stimulation. Stimulation was not universally beneficial and changing the waveform, frequency, or intensity of the field affected gene expression levels. Additionally, different cell donors had variable responses to the same stimulation profile. This finding supports patient-by-patient optimization, whereby a personalized microenvironment stimulation target is generated based on a patient profile and then actively modified and optimized subject to pain level feedback from the patient.

### Materials and Methods

### Astrocytes as a Model for Pain

The sensation of pain is a complex biophysical process which involves many neuroanatomic and neurochemical systems. Primarily, the nociceptive pathway is used to transmit and process information to and from the brain upon noxious stimulation of tissue. On a macroscopic level, this involves the transmission of signals from the area of noxious stimuli using the afferent pathway to the dorsal root ganglion which transfers the information to the brain.

Within the nociceptive pathway, there is a known interaction between neuronal cells and neuroglial cells which contribute to the perception of pain. As the most abundant cell type in the CNS, astrocytes have been identified as an active contributor to the sensation of pain through the process of reactive astrogliosis. In the presence of noxious stimuli, astrocytes undergo a phenotypic and functional change to become reactive which involves inflammatory and neurotoxic responses contributing to the sensation of pain. In particular, cortical reactive astrocytes have demonstrated the ability to create a chemical imbalance of glutamate and gamma aminobutyric acid (GABA) which leads to synaptic remodelling and chronic pain. Thus, the induction of astrocytes into a reactive state may be considered a reasonable model of pain by examining the ability of EMF to revert reactive astrocytes into a naïve or non-inflammatory state.

### Cell Donors—Normal Human Astrocytes

Normal human astrocytes (NHA), isolated from brain tissue (cerebral cortex), from three different donors were purchased. The first donor, NHA1, was procured from Lonza (Lonza Walkersville, Walkersville, MD, USA), while NHA2 and NHA3 were purchased from ScienCell (ScienCell Research Laboratories, Inc., Carlsbad, CA, USA). Information pertaining to the donors themselves was not provided, but baseline gene expression levels (see below) reveal inter-donor differences prior to any EMF treatment.

### Cell Culture

Astrocytes were sourced from different donors (Lonza: CC-2565/ScienCell: 1800). Cells were thawed from frozen (−150° C.) and plated into a poly-D-lysine coated T75 flask containing astrocyte culture medium (Lonza: CC-3186/ScienCell: 1801-prf). Cells were placed in an incubator set to 37° C. and 5.0% CO2. Media was exchanged 1 day after thawing to remove the DMSO. Media was exchanged every 2-3 days thereafter. Cells proliferated until 70-90% confluency. Upon reaching this threshold, cells were washed with DPBS and detached using 0.05% Trypsin supplemented with neutral proteases. The detached cells were neutralized, centrifuged, and seeded in poly-D-lysine coated culture vessels at a concentration of 5000 cells/cm² for passaging.

For PCR analysis, the dissociated cells (approximately 500,000 cells) were neutralized using an equal volume of complete media and centrifuged for 2 minutes at 4000 RPM. Following centrifugation, the supernatant was aspirated, and the cell pellets were immediately frozen at −80° C. for future RNA extraction.

### Reactive State Growth Factors

To induce astrocytes into an inflammatory reactive state, cytokines TNFα (R&D Systems: 210-TA-005/CF) and IL-1B (201-LB-005/CF) were added to cultures at a concentration of 10 ng/mL following the method described by Hyvärinen et al., 2019 (Hyvärinen T, Hagman S, et al., Sci Rep. 2019; 9:16944). Media was exchanged every 2-3 days.

### Electromagnetic Field Stimulation

Cells were exposed, as previously described for mesenchymal stem cells, to a spatially uniform, time-varying magnetic field using Helmholtz coils. The exposure system (coils, signal generator, incubators, etc.) is as described in Example 1 (bone healing).

Previous pain management systems have implemented a diverse set of stimulation profiles using a variety of deployment methods. There is no consensus on the most effective EMF parameters but there is a trend towards low intensity and very low frequency stimulation that results in an imperceptible electromagnetic field. Table 5 contains the exposure parameters used in each of three experiments designed to show the inter-donor and intra-donor variability of astrocytes exposed to electromagnetic stimulation. Experiment 1 tested the response of all three donors to a 15 Hz sinusoid for 10 min/day, while Experiments 2 and 3 only included a single donor (NHA2). Relative to the sinusoid, the ramp (Experiment 2) and pulse (Experiment 3) functions induce stronger electric currents in the media due to sharp changes in the input signal, and do so at significantly higher and lower frequencies, respectively.

TABLE 5
Exposure parameters for normal human astrocyte experiments

| | | | | | Daily | Exposure |
|---|---|---|---|---|---|---|
| | | | B | f | exposure | length |
| Experiment | Donors | Waveform | (mT) | (Hz) | (minutes) | (days) |
| 1 | ALL | Sine | 1 | 15 | 10 | 2 |
| 2 | NHA2 | Ramp | 1 | 150 | 240 | 2 |
| | only | | | | | |
| 3 | NHA2 | Pulse | 2 | 2 | 60 | 4 |
| | only | | | | | |

### Imaging

Cells were imaged prior to each feed and on the day of termination using a Zeiss Axio Vert A1 Inverted Microscope at 50× magnification.

qPCR

RNA was extracted from frozen cell pellets using a RNeasy kit (Qiagen: 74004) and subsequent cDNA was reverse transcribed (Qiagen: 330404). The expression levels of glial fibrillary acidic protein (GFAP), interleukin 6 (IL6), interleukin 1β (IL-1β), tumor necrosis factor-α (TNFα), complement component 3 (C3), transforming growth factor Beta 1 (TGFβ1), signal transducer and activator of transcription (STAT3), interleukin 8 (IL8), SRY-box transcription factor 9 (SOX9) were determined relative to the reference gene glyceraldehyde 3-phosphate dehydrogenase (GAPDH) by real-time RT-PCR using the SYBR Green detection system. Each sample was diluted 1:10 and analyzed in duplicate using the iTaq Universal SYBR Green Supermix (BioRad: 1725122) with the optimized concentration of forward and reverse primer (0.6 μM) on a CFX96 Touch™ real-time RT-PCR machine (Bio Rad). The program used to run all samples included an enzyme activation step at 95° C. for 30 sec followed by 40 cycles with 95° C. for 3 sec and 60, 61, 62, or 63° C. (depending on target gene) for 30 sec. After the amplification phase, a dissociation curve was established to ensure the presence of a single amplicon. Reaction efficiencies were 100±10% with an R 2>0.990 and calculated by the CFX Manager Software (Bio Rad, Mississauga, ON, Canada). In each assay, a standard curve created with gBlocks (specifically designed for each gene amplicon), a no template control, and a no reverse transcription control (to ensure the absence of genomic DNA in the samples) were run with the samples. The standard curve was generated by serial dilution of the gBlocks. The standard curve was then used to interpolate and calculate the mRNA level of target and reference gene in each sample. The mRNA level of each target gene was calculated relative to the reference gene GAPDH.

### PCR Array

RNA was extracted from frozen cell pellets using a RNeasy kit (Qiagen: 74004) and subsequent cDNA was reverse transcribed (Qiagen: 330404). Reverse transcribed templates ware analyzed by a Human Pain: Neuropathic and Inflammatory PCR Array (Human Pain: Neuropathic and Inflammatory RT² Profiler Array (Qiagen: 330231; PAHS-162Z)) on a BioRad CFX96 Real Time PCR Detection System. Each experimental point was performed in triplicate. Gene expression of the 84 genes of interest on each PCR array was normalized to reference genes (ΔCₜ) and then averaged within an experimental group. ΔΔCₜ for each gene was then calculated between groups before fold change was calculated according to 2^{(-ΔΔCt)}. In the results below, some data is presented in the form of heatmaps, where the fold change is displayed at log base 2.

### Results

### Morphology of Astrocyte Phenotypes

Control and Exposed donor cell culture groups were imaged before each feed and before termination to monitor morphology. For groups receiving reactive state cytokines (IL-1β and TNFα), there is an expected morphological change as the astrocytes alter their phenotype. FIG. 21 demonstrates this difference in morphology whereby non-reactive cells (A) are more filamentous while reactive cells (B) adopt a more polygonal morphology.

### Placing Astrocytes Into A Reactive State

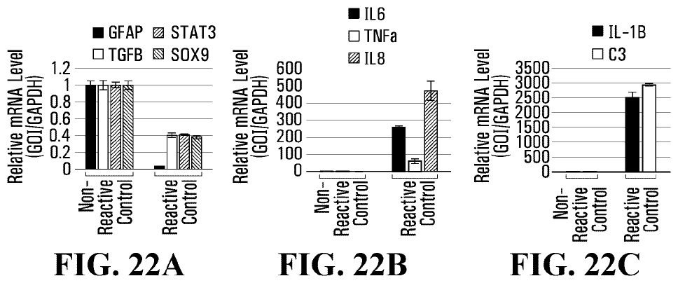

The normal human astrocytes were placed into their reactive state by culturing with an additional set of growth factors, including IL-1β and TNFα. The state of the astrocytes was verified 72 hours after seeding by terminating the cells, isolating RNA and detecting for specific marker genes using qPCR. Nine genes were quantified as markers of the reactive state, including four that should be down-regulated (GFAP, TGFβ, STAT3 and SOX9) and five that are expected to have increase expression levels (IL6, TNFα, IL8, IL-1β and C3). FIG. 22 plots the expression level of each gene for a Donor NHA2 cell group. The expected trends were observed, and the reactive state of the astrocytes is confirmed. The reactive (or pain) state exhibits low levels of GFAP, TGFB, STAT3 and SOX9, and elevated expression of IL6, TNFα, IL8, IL-1β and C3, compared to non-reactive control levels.

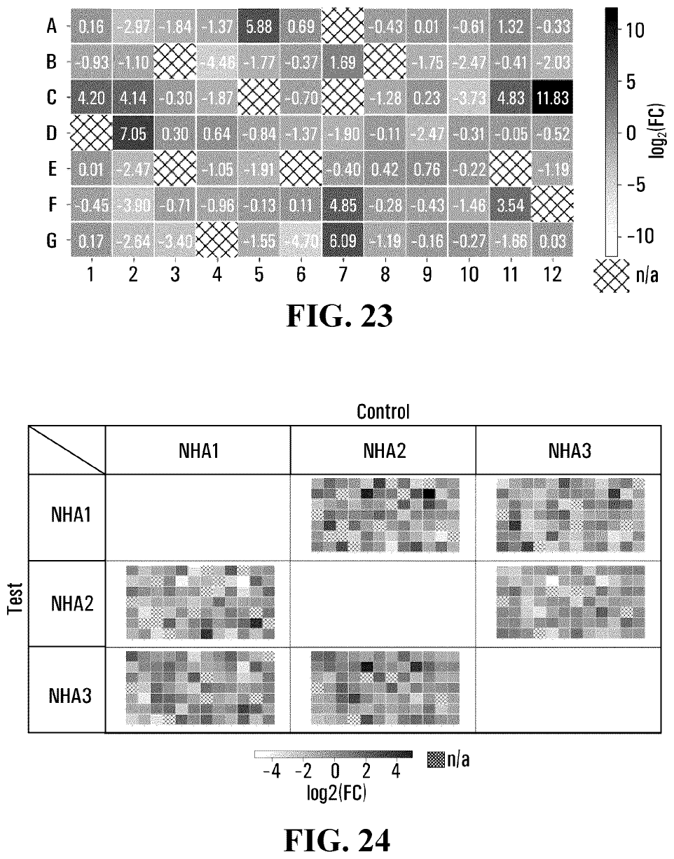

The PCR array heatmap in FIG. 23 shows the expression levels of a selection of genes, curated for pain and neuroinflammation, in reactive astrocytes (compared to non-reactive astrocytes). The gene array contains 84 genes (Appendix III). Inflammatory cytokines IL-1β (position C12) and IL6 (D2) are both highly expressed when the cells are reactive and is indicative of a neuroinflammatory state. The goal of EMF exposure is to counter the elevated expression of genes belonging to inflammatory pain signalling pathways and, hence, bring expression levels back towards those of the non-reactive state.

### Baseline Expression Of Reactive Astrocytes

The pain PCR array was utilized to compare the baseline gene expression levels of each donor cell group to the others while in a reactive state. FIG. 24 shows the heatmaps, where one donor is used as a test subject and the other a calibrator. Up-regulation or down-regulation between donors indicates higher or lower baseline expression, respectively. These differences derive from, and are incorporated into, the patient profile and the transition from non-reactive to reactive state and may affect cellular response to EMF stimulation. In comparing any two donors, there are strong differences (up or down) from one to another, but there are no biases.

### Inter-Donor Differences In Response To EMF Exposure

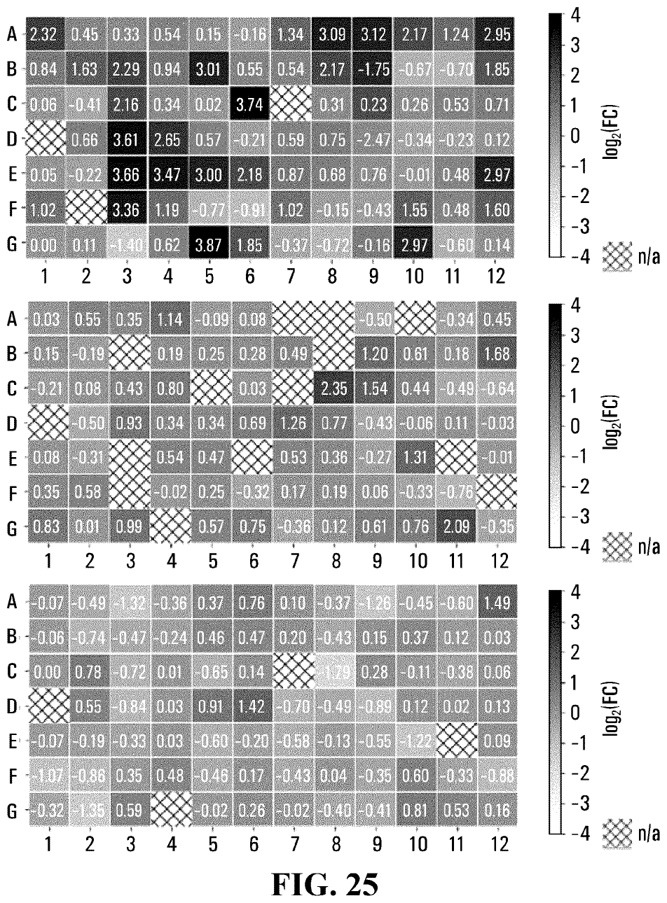

The stimulation profile tested in Experiment 1 (Table 4) was applied to each astrocyte donor cell group to demonstrate inter-donor variability due to EMF exposure. The short daily exposure produces quantifiable changes in gene expression levels and notable changes from donor to donor (relative to their own controls). FIG. 25 contains heatmaps corresponding to each donor that demonstrate significantly different responses to the same exposure. NHA1 responded with increased gene expression in a majority of the array, including many inflammatory markers that were already highly up-regulated by the change to a reactive state. To the contrary, many of the same genes that are up-regulated in NHA1 are down-regulated in NHA3. It is apparent that a single stimulation profile is unlikely to suit every donor and personalization will be required.

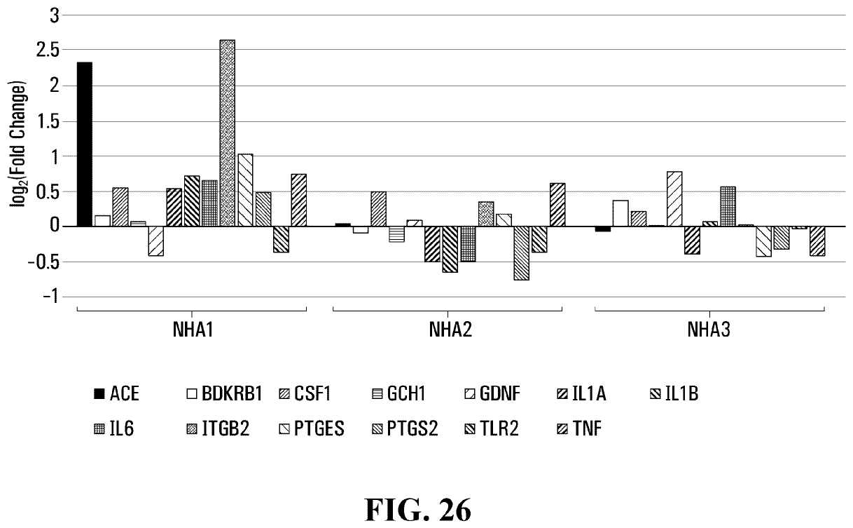

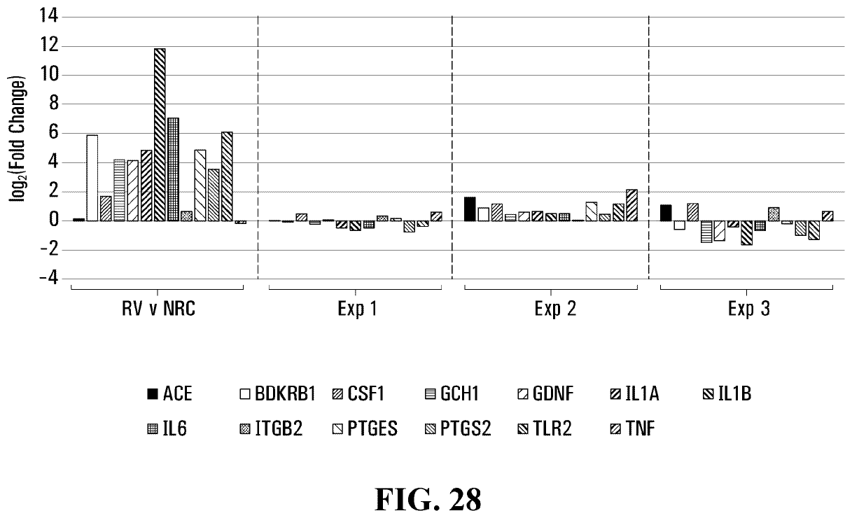

In FIG. 26, fold change values for select genes are plotted for each donor relative to unexposed reactive cells from the same donor group. All these genes are involved in pain response modulation and are, specifically, along inflammatory response pathways. The thirteen genes highlighted in FIG. 28 are described in Appendix III. Astrocytes are active participants in these pathways when in a reactive state and gene expression is increased in this pain condition. The objective of EMF treatment is to counter the changes made by the (reactive) growth factors and have expression levels trend back towards non-reactive measurements. The exposure caused up-regulated expression of almost all genes in NHA1 and suggests that the stimulation was pro-inflammatory in that donor. The exposure had a more favourable response from NHA3 but inflammatory markers IL-1β and IL6 are up-regulated—indicative of a reactive astrocyte. The stimulation was best suited to NHA2 which responded with mostly down-regulated gene expression (relative to the unstimulated reactive cells), particularly in the important inflammatory markers: IL-1β and IL6.

### Intra-Donor Variability Demonstrated By Varying EMF Treatment Parameters

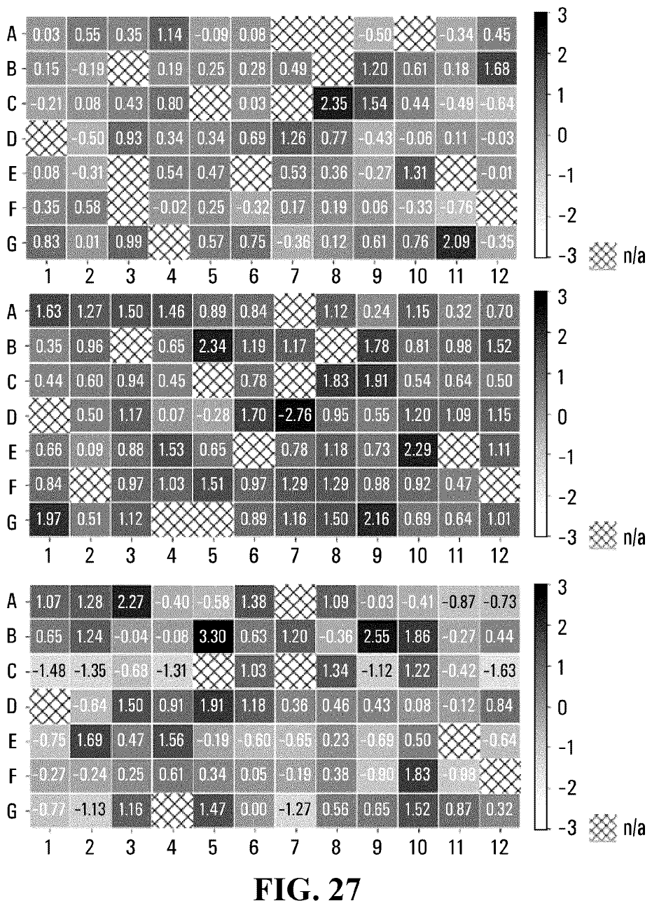

Donor NHA2 was exposed to three different EMF stimulation profiles covering a broad range of frequencies, exposure times, and signal shapes (Table 4). The three experiments each resulted in altered gene expression (relative to reactive state quantities) and demonstrate the varied effects treatment with EMF can elicit. Heatmaps in FIG. 27 show gene expression changes for 84 unique genes as a result of the three different stimulation profiles. It is immediately apparent that the higher frequency signal used in Experiment 2 increased gene expression for almost every gene, which would further exacerbate the (pain) issue. Experiment 1 and 3 produced more desirable responses as many pain-associated genes are down-regulated and many others shift back towards the level of non-reactive astrocytes.

FIG. 28 shows 13 inflammatory genes affected by the induced electric currents of Experiments 1-3. Gene expression levels are modulated relative to a reactive control. For reference, the reactive versus non-reactive controls comparison is also included. In Experiments 1 and 3 most of the genes that were strongly up-regulated by the change in reactivity are now exhibiting decreased expression after only two to four short exposures. IL-1β and IL6, in particular, are markers of the change back towards a non-reactive state, and both are down-regulated in Experiment 1 and Experiment 3 (strongest effect). These two experiments, despite utilizing significantly different EMF parameters, affect the same genes in the same directions, but Experiment 2 increases expression of all the genes. It is apparent that positive and negative outcomes can be identified for the same donor with minor changes to the electromagnetic field (while different, each EMF is in the extremely low frequency range and is imperceptible to the patient) and that the therapy must be tailored to the patient to achieve the optimal outcome.

### Example Summary

The result of Examples 1-3 when taken together, demonstrate inter-donor differences at the cellular level supporting the need for donor-to-donor treatment personalization and optimization.

The results also support the use of gene expression as a useful tool to monitor and characterize the effects of EMF treatment on the cells of the microenvironment and may further provide guidance for either amending the treatment protocol or for the use of an adjunct therapy. A baseline gene expression profile can be obtained from patient cells prior to the start of treatment to determine EMF gene targeting effects. This clinical data would be supplemental data to include in the “Patient Profile” as one of the variable biological parameters fed to MiCE to compute a personalized microenvironment stimulation target for the patient. Any appropriate cell type may be extracted/harvested from a patient from for example a blood draw, a mouth swab, or invasively from the microenvironment. Gene expression and control levels can be assessed at different time points throughout the treatment protocol to allow progressive therapy optimization. The selection of genes may be dependent on the cell/tissue type as well as on the type of injury and disease. Non-limiting examples of genes for expression array assessment are listed in Appendices I-III.

### Appendices

APPENDIX I
Genes for Human Mesenchymal Stem Cell PRC array

| Array Code | Gene Label | Gene Name |
|---|---|---|
| A01 | ABCB1 | ATP-bindA4:A29ing cassette, sub-family B (MDR/TAP), member 1 |
| A02 | ACTA2 | Actin, alpha 2, smooth muscle, aorta |
| A03 | ALCAM | Activated leukocyte cell adhesion molecule |
| A04 | ANPEP | Alanyl (membrane) aminopeptidase |
| A05 | ANXA5 | Annexin A5 |
| A06 | BDNF | Brain-derived neurotrophic factor |
| A07 | BGLAP | Bone gamma-carboxyglutamate (gla) protein |
| A08 | BMP2 | Bone morphogenetic protein 2 |
| A09 | BMP4 | Bone morphogenetic protein 4 |
| A10 | BMP6 | Bone morphogenetic protein 6 |
| A11 | BMP7 | Bone morphogenetic protein 7 |
| A12 | CASP3 | Caspase 3, apoptosis-related cysteine peptidase |
| B01 | CD44 | CD44 molecule (Indian blood group) |
| B02 | COL1A1 | Collagen, type I, alpha 1 |
| B03 | CSF2 | Colony stimulating factor 2 (granulocyte-macrophage) |
| B04 | CSF3 | Colony stimulating factor 3 (granulocyte) |
| B05 | CTNNB1 | Catenin (cadherin-associated protein), beta 1, 88 kDa |
| B06 | EGF | Epidermal growth factor |
| B07 | ENG | Endoglin |
| B08 | ERBB2 | V-crb-b2 erythroblastic leukemia viral oncogene homolog 2, neuro/glioblastoma |
| | | derived oncogene homolog (avian) |
| B09 | FGF10 | Fibroblast growth factor 10 |
| B10 | FGF2 | Fibroblast growth factor 2 (basic) |
| B11 | FUT1 | Fucosyltransferase 1 (galactoside 2-alpha-L-fucosyltransferase, H blood group) |
| B12 | FUT4 | Fucosyltransferase 4 (alpha (1,3) fucosyltransferase, myeloid-specific) |
| C01 | FZD9 | Frizzled family receptor 9 |
| C02 | GDF15 | Growth differentiation factor 15 |
| C03 | GDF5 | Growth differentiation factor 5 |
| C04 | GDF6 | Growth differentiation factor 6 |
| C05 | GDF7 | Growth differentiation factor 7 |
| C06 | GTF3A | General transcription factor IIIA |
| C07 | HAT1 | Histone acetyltransferase 1 |
| C08 | HDAC1 | Histone deacetylase 1 |
| C09 | HGF | Hepatocyte growth factor (hepapoietin A; scatter factor) |
| C10 | HNF1A | HNF1 homeobox A |
| C11 | ICAM1 | Intercellular adhesion molecule |
| C12 | IFNG | Interferon, gamma |
| D01 | IGF1 | Insulin-like growth factor 1 (somatomedin C) |
| D02 | IL10 | Interleukin 10 |
| D03 | IL1B | Interleukin 1, beta |
| D04 | IL6 | Interleukin 6 (interferon, beta 2) |
| D05 | INS | Insulin |
| D06 | ITGA6 | Integrin, alpha 6 |
| D07 | ITGAV | Integrin, alpha V (vitronectin receptor, alpha polypeptide, antigen CD51) |
| D08 | ITGAX | Integrin, alpha X (complement component 3 receptor 4 subunit) |
| D09 | ITGB1 | Integrin, beta 1 (fibronectin receptor, beta polypeptide, antigen CD29 includes |
| | | MDF2, MSK12) |
| D10 | JAG1 | Jagged 1 |
| D11 | KAT2B | K(lysine) acetyltransferase 2B |
| D12 | KDR | Kinase insert domain receptor (a type III receptor tyrosine kinase) |
| E01 | KITLG | KIT ligand |
| E02 | LIF | Leukemia inhibitory factor (cholinergic differentiation factor) |
| E03 | MCAM | Melanoma cell adhesion molecule |
| E04 | MMP2 | Matrix metallopeptidase 2 (gelatinase A, 72 kDa gelatinase, 72 kDa type IV |
| | | collagenase) |
| E05 | NES | Nestin |
| E06 | NGFR | Nerve growth factor receptor |
| E07 | NOTCH1 | Notch 1 |
| E08 | NT5E | 5′-nucleotidase, ecto (CD73) |
| E09 | NUDT6 | Nudix (nucleoside diphosphate linked moiety X)-type motif 6 |
| E10 | PDGFRB | Platelet-derived growth factor receptor, beta polypeptide |
| E11 | PIGS | Phosphatidylinositol glycan anchor biosynthesis, class S |
| E12 | POU5F1 | POU class 5 homeobox 1 |
| F01 | PPARG | Peroxisome proliferator-activated receptor gamma |
| F02 | PROM1 | Prominin 1 |
| F03 | PTK2 | PTK2 protein tyrosine kinase 2 |
| F04 | PTPRC | Protein tyrosine phosphatase, receptor type, C |
| F05 | RHOA | Ras homolog gene family, member A |
| F06 | RUNX2 | Runt-related transcription factor 2 |
| F07 | SLC17A5 | Solute carrier family 17 (anion/sugar transporter), member 5 |
| F08 | SMAD4 | SMAD family member 4 |
| F09 | SMURF1 | SMAD specific E3 ubiquitin protein ligase 1 |
| F10 | SMURF2 | MAD specific E3 ubiquitin protein ligase 2 |
| F11 | SOX2 | SRY (sex determining region Y)-box 2 |
| F12 | SOX9 | SRY (sex determining region Y)-box 9 |
| G01 | TBX5 | T-box 5 |
| G02 | TERT | Telomerase reverse transcriptase |
| G03 | TGFB1 | Transforming growth factor, beta 1 |
| G04 | TGFB3 | Transforming growth factor, beta 3 |
| G05 | THY1 | Thy-1 cell surface antigen |
| G06 | TNF | Tumor necrosis factor |
| G07 | VCAM1 | Vascular cell adhesion molecule 1 |
| G08 | VEGFA | Vascular endothelial growth factor A |
| G09 | VIM | Vimentin |
| G10 | VWF | Von Willebrand factor |
| G11 | WNT3A | Wingless-type MMTV integration site family, member 3A |
| G12 | ZFP42 | Zinc finger protein 42 homolog (mouse) |

Osteogenic genes selected from the MSC PCR array

| Array | Gene | |
|---|---|---|
| Code | Label | Description (osteogenic genes) |
| A08 | BMP2 | BMP2 plays an important role in bone and cartilage formation via the hedgehog pathway |
| B09 | FGF10 | FGF signalling plays essential roles in bone formation and is vital for bone homeostasis |
| C08 | HDAC1 | HDAC1 is abundant in bone and articular cartilage |
| C10 | HNF1A | No known function in bone healing |
| D12 | KDR | Indirectly promotes bone formation by complementing and potentiating the osteogenic effect of |
| | | other growth factors. |
| F03 | PTK2 | PTK2 Plays a role in osteogenesis and differentiation of osteoblasts |
| F06 | RUNX2 | RUNX2 activates differentiation of multipotent mesenchymal cells into immature osteoblasts. |
| | | RUNX2 simultaneously inhibits the formation of mature bone |
| F09 | SMURF1 | SMURF1 mediates protein degradation of the osteoblast-specific transcription factor |
| | | RUNX2/CBFA1 |
| F10 | SMURF2 | SMURF2 is a regulator in osteoblast-dependent osteoclast activity, and maintains bone |
| | | homeostasis |
| G01 | TBX5 | TBX5 promotes bone growth and maturation by controlling via Cx40 the expression of SOX9 |

Chondrogenic genes selected from the MSC PCR array

| Array | Gene | |
|---|---|---|
| Code | Label | Description (chondrogenic genes) |
| A01 | ABCB1 | P-gp expression has been shown to enhance osteogenesis |
| A08 | BMP2 | BMP2 plays an important role in bone and cartilage formation via the hedgehog pathway |
| A09 | BMP4 | Regulates cellular lineage commitment, morphogenesis, differentiation, proliferation, and |
| | | apoptosis. |
| C03 | GDF5 | GDF5 plays an integral role in bone and cartilage |
| C07 | HAT1 | Higher expression of HAT1 is associated with better quantity and quality of bone tissue |
| D11 | KAT2B | KAT2B is necessary for the growth and differentiation of craniofacial cartilage and bone |
| F12 | SOX9 | SOX9 is essential for chondrocyte differentiation and cartilage formation |
| G03 | TGFB1 | TGF-β1 plays a major role in development and maintenance of skeletal tissue, balancing bone |
| | | resorption, and bone formation |

APPENDIX II
Genes for Cancer PathwayFinder PCR array

| Array | Gene | |
|---|---|---|
| Code | Label | Gene Name |
| A01 | ACLY | ATP citrate lyase |
| A02 | ACSL4 | Acyl-CoA synthetase long-chain family member 4 |
| A03 | ADM | Adrenomedullin |
| A04 | ANGPT1 | Angiopoietin 1 |
| A05 | ANGPT2 | Angiopoietin 2 |
| A06 | APAF1 | Apoptotic peptidase activating factor 1 |
| A07 | ARNT | Aryl hydrocarbon receptor nuclear translocator |
| A08 | ATP5F1A | ATP synthase F1 subunit alpha |
| A09 | AURKA | Aurora kinase A |
| A10 | BCL2L11 | BCL2-like 11 (apoptosis facilitator) |
| A11 | BIRC3 | Baculoviral IAP repeat containing 3 |
| A12 | BMI1 | BMI1 polycomb ring finger oncogene |
| B01 | CA9 | Carbonic anhydrase 1X |
| B02 | CASP2 | Caspase 2, apoptosis-related cysteine peptidase |
| B03 | CASP7 | Caspase 7, apoptosis-related cysteine peptidase |
| B04 | CASP9 | Caspase 9, apoptosis-related cysteine peptidase |
| B05 | CCL2 | Chemokine (C-C motif) ligand 2 |
| B06 | CCND2 | Cyclin D2 |
| B07 | CCND3 | Cyclin D3 |
| B08 | CDC20 | Cell division cycle 20 homolog (S. cerevisiae) |
| B09 | CDH2 | Cadherin 2, type 1, N-cadherin (neuronal) |
| B10 | CFLAR | CASP8 and FADD-like apoptosis regulator |
| B11 | COX5A | Cytochrome c oxidase subunit Va |
| B12 | CPT2 | Carnitine palmitoyltransferase 2 |
| C01 | DDB2 | Damage-specific DNA binding protein 2, 48 kDa |
| C02 | DDIT3 | DNA-damage-inducible transcript 3 |
| C03 | DKC1 | Dyskeratosis congenita 1, dyskerin |
| C04 | DSP | Desmoplakin |
| C05 | E2F4 | E2F transcription factor 4, p107/p130-binding |
| C06 | EPO | Erythropoietin |
| C07 | ERCC3 | Excision repair cross-complementing rodent repair deficiency, complementation group 3 (xeroderma |
| | | pigmentosum group B complementing) |
| C08 | ERCC5 | Excision repair cross-complementing rodent repair deficiency, complementation group 5 |
| C09 | ETS2 | V-Ets erythroblastosis virus E26 oncogene homolog 2 (avian) |
| C10 | FASLG | Fas ligand (TNF superfamily, member 6) |
| C11 | FGF2 | Fibroblast growth factor 2 (basic) |
| C12 | FLT1 | Fms-related tyrosine kinase 1 (vascular endothelial growth factor/vascular permeability factor receptor) |
| D01 | FOXC2 | Forkhead box C2 (MFH-1, mesenchyme forkhead 1) |
| D02 | G6PD | Glucose-6-phosphate dehydrogenase |
| D03 | GADD45G | Growth arrest and DNA-damage-inducible, gamma |
| D04 | GPD2 | Glycerol-3-phosphate dehydrogenase 2 (mitochondrial) |
| D05 | GSC | Goosecoid homeobox |
| D06 | HMOX1 | Heme oxygenase (decycling) 1 |
| D07 | IGFBP3 | Insulin-like growth factor binding protein 3 |
| D08 | IGFBP5 | Insulin-like growth factor binding protein 5 |
| D09 | IGFBP7 | Insulin-like growth factor binding protein 7 |
| D10 | KDR | Kinase insert domain receptor (a type III receptor tyrosine kinase) |
| D11 | KRT14 | Keratin 14 |
| D12 | LDHA | Lactate dehydrogenase A |
| E01 | LIG4 | Ligase IV, DNA, ATP-dependent |
| E02 | LPL | Lipoprotein lipase |
| E03 | MAP2K1 | Mitogen-activated protein kinase kinase 1 |
| E04 | MAP2K3 | Mitogen-activated protein kinase kinase 3 |
| E05 | MAPK14 | Mitogen-activated protein kinase 14 |
| E06 | MCM2 | Minichromosome maintenance complex component 2 |
| E07 | MKI67 | Antigen identified by monoclonal antibody Ki-67 |
| E08 | NOL3 | Nucleolar protein 3 (apoptosis repressor with CARD domain) |
| E09 | OCLN | Occludin |
| E10 | PFKL | Phosphofructokinase, liver |
| E11 | PGF | Placental growth factor |
| E12 | PINX1 | PIN2/TERF1 interacting, telomerase inhibitor 1 |
| F01 | POLB | Polymerase (DNA directed), beta |
| F02 | PPP1R15A | Protein phosphatase 1, regulatory (inhibitor) subunit 15A |
| F03 | SERPINB2 | Serpin peptidase inhibitor, clade B (ovalbumin), member 2 |
| F04 | SERPINF1 | Serpin peptidase inhibitor, clade F (alpha-2 antiplasmin, pigment epithelium derived factor), member 1 |
| F05 | SKP2 | S-phase kinase-associated protein 2 (p45) |
| F06 | SLC2A1 | Solute carrier family 2 (facilitated glucose transporter), member 1 |
| F07 | SNAI1 | Snail homolog 1 (Drosophila) |
| F08 | SNAI2 | Snail homolog 2 (Drosophila) |
| F09 | SNAI3 | Snail homolog 3 (Drosophila) |
| F10 | SOD1 | Superoxide dismutase 1, soluble |
| F11 | SOX10 | SRY (sex determining region Y)-box 10 |
| F12 | STMN1 | Stathmin 1 |
| G01 | TBX2 | T-box 2 |
| G02 | TEK | TEK tyrosine kinase, endothelial |
| G03 | TEP1 | Telomerase-associated protein 1 |
| G04 | TERF1 | Telomeric repeat binding factor (NIMA-interacting) 1 |
| G05 | TERF2IP | Telomeric repeat binding factor 2, interacting protein |
| G06 | TINF2 | TERF1 (TRF1)-interacting nuclear factor 2 |
| G07 | TNKS | Tankyrase, TRF1-interacting ankyrin-related ADP-ribose polymerase |
| G08 | TNKS2 | Tankyrase, TRF1-interacting ankyrin-related ADP-ribose polymerase 2 |
| G09 | UQCRFS1 | Ubiquinol-cytochrome c reductase, Rieske iron-sulfur polypeptide 1 |
| G10 | VEGFC | Vascular endothelial growth factor C |
| G11 | WEE1 | WEE1 homolog (S. pombe) |
| G12 | XIAP | X-linked inhibitor of apoptosis |

List of functions of key genes in cancer-relevant pathways.

| Array | Gene | |
|---|---|---|
| Code | Label | Description (role in cancer pathways) |
| A04 | ANGPT1 | Involved in both angiogenesis and maintenance of vascular stability. Promotes endothelial cell survival through |
| | | activation of Tie2. |
| A06 | APAF1 | Part of the intrinsic apoptotic pathway. Forms the apoptosome with cytochrome c and dATP. The apoptosome |
| | | activates caspase 9 resulting in a caspase cascade and cell death. Might also play a role in chemosensitivity. |
| B02 | CASP2 | Part of the apoptosis pathway. Caspase 2 is an initiator caspase and activates effector caspases as part of the |
| | | caspase cascade leading to cell death. |
| B05 | CCL2 | A member of the CC chemokine family that binds the CCR2 receptor and acts as a chemoattractant for a variety of |
| | | immune cells and is involved in the inflammatory response and angiogenesis. Increased expression is associated |
| | | with tumor progression and metastasis in numerous cancers. |
| C01 | DDB2 | Recognizes and binds to damaged DNA and, along with DDB1, forms a UV-damaged DNA binding protein |
| | | complex (UV-DDB). This complex mediates recruitment of proteins for nucleotide excision repair. |
| D03 | GADD45G | GADD45G is one of three GADD45 isoforms, all of which are induced by cellular stress. GADD45G is involved in |
| | | the regulation of apoptosis and growth arrest including through activation of the p38/JNK pathway via |
| | | MTK1/MEKK4 kinase. Is often down regulated in cancer by NF-κB to inhibit apoptosis. |
| D07 | IGFBP3 | Binds IGF (Insulin-like growth factor) as it circulates in the blood and regulates bioavailability. Independent of |
| | | IGF, IGFBP3 has many interaction partners and is involved in cell cycle arrest and apoptosis, as well as DNA |
| | | repair and cell survival. |
| D09 | IGFBP7 | IGFBP7 is a member of a subfamily of low-affinity IGFBPs (bind IFGs with lower affinity). Can function as a |
| | | tumor suppresser by reducing cell growth including through interaction with members of the TGF-β superfamily. |
| E04 | MAP2K3 | Part of the p38 MAPK cascade, which induces cell cycle arrest. Under conditions of cell stress, MAP2K3 is |
| | | activated by an upstream MAP3K enabling it to phosphorylate and activate downstream MAPKs including |
| | | MAPK14/p38α. |
| E05 | MAPK14 | Also known as p38α. Part of the p38 MAPK cascade, which induces cell cycle arrest. During conditions of cell |
| | | stress MAPK14 is phosphorylated and activated by MAP2K3. |
| E06 | MCM2 | MCM2 forms a heterohexametric protein ring with MCM3-7 that is required for initiation and elongation of DNA |
| | | replication. |
| E07 | MKI67 | Binds DNA and helps maintain individual chromosomes during mitosis after the nuclear envelope has been |
| | | dissolved. Used as a marker of proliferation in tumor cells as it is only expressed in the nucleus |
| | | of proliferating cells. |
| E08 | NOL3 | Also called Apoptosis Repressor with CARD Domain (ARC). An apoptosis repressor. Can prevent apoptosis |
| | | through both the intrinsic and extrinsic pathways. Has many interactors including caspase 8. |
| F02 | PPP1R15A | Also known as Growth arrest and DNA damage-inducible protein 34 (GADD34). Expression is increased as part of |
| | | the integrated stress response. PPP1R15A recruits protein phosphatase 1 to dephosphorylate eIF2α, removing the |
| | | inhibition of protein translation. Overexpression can mediate apoptosis in response to DNA damage |
| | | or cellular stress. |
| G12 | XIAP | Represses apoptosis through both the intrinsic and extrinsic pathways, including through interaction |
| | | with several caspases. |

APPENDIX III
Genes for Pain Neuropathic and Inflammatory PCR array

| Array | Gene | |
|---|---|---|
| Code | Label | Gene Name |
| A01 | ACE | Angiotensin I converting enzyme (peptidyl-dipeptidase A) 1 |
| A02 | ADORA1 | Adenosine A1 receptor |
| A03 | ADRB2 | Adrenergic, beta-2-, receptor, surface |
| A04 | ALOX5 | Arachidonate 5-lipoxygenase |
| A05 | BDKRB1 | Bradykinin receptor B1 |
| A06 | BDNF | Brain-derived neurotrophic factor |
| A07 | CACNA1B | Calcium channel, voltage-dependent, N type, alpha 1B subunit |
| A08 | CALCA | Calcitonin-related polypeptide alpha |
| A09 | CCK | Cholecystokinin |
| A10 | CCKBR | Cholecystokinin B receptor |
| A11 | CCL2 | Chemokine (C-C motif) ligand 2 |
| A12 | CCR2 | Chemokine (C-C motif) receptor 2 |
| B01 | CD200 | CD200 molecule |
| B02 | CD4 | CD4 molecule |
| B03 | CHRNA4 | Cholinergic receptor, nicotinic, alpha 4 |
| B04 | CNR1 | Cannabinoid receptor 1 (brain) |
| B05 | CNR2 | Cannabinoid receptor 2 (macrophage) |
| B06 | COMT | Catechol-O-methyltransferase |
| B07 | CSF1 | Colony stimulating factor 1 (macrophage) |
| B08 | CX3CR1 | Chemokine (C-X3-C motif) receptor 1 |
| B09 | DBH | Dopamine beta-hydroxylase (dopamine beta-monooxygenase) |
| B10 | EDN1 | Endothelin 1 |
| B11 | EDNRA | Endothelin receptor type A |
| B12 | FAAH | Fatty acid amide hydrolase |
| C01 | GCH1 | GTP cyclohydrolase 1 |
| C02 | GDNF | Glial cell derived neurotrophic factor |
| C03 | GRIN1 | Glutamate receptor, ionotropic, N-methyl D-aspartate 1 |
| C04 | GRIN2B | Glutamate receptor, ionotropic, N-methyl D-aspartate 2B |
| C05 | GRM1 | Glutamate receptor, metabotropic 1 |
| C06 | GRM5 | Glutamate receptor, metabotropic 5 |
| C07 | HTR1A | 5-hydroxytryptamine (serotonin) receptor 1A |
| C08 | HTR2A | 5-hydroxytryptamine (serotonin) receptor 2A |
| C09 | IL10 | Interleukin 10 |
| C10 | IL18 | Interleukin 18 (interferon-gamma-inducing factor) |
| C11 | IL1A | Interleukin 1, alpha |
| C12 | IL1B | Interleukin 1, beta |
| D01 | IL2 | Interleukin 2 |
| D02 | IL6 | Interleukin 6 (interferon, beta 2) |
| D03 | ITGAM | Integrin, alpha M (complement component 3 receptor 3 subunit) |
| D04 | ITGB2 | Integrin, beta 2 (complement component 3 receptor 3 and 4 subunit) |
| D05 | KCNIP3 | Kv channel interacting protein 3, calsenilin |
| D06 | KCNJ6 | Potassium inwardly-rectifying channel, subfamily J, member 6 |
| D07 | KCNQ2 | Potassium voltage-gated channel, KQT-like subfamily, member 2 |
| D08 | KCNQ3 | Potassium voltage-gated channel, KQT-like subfamily, member 3 |
| D09 | MAOB | Monoamine oxidase B |
| D10 | MAPK1 | Mitogen-activated protein kinase 1 |
| D11 | MAPK14 | Mitogen-activated protein kinase 14 |
| D12 | MAPK3 | Mitogen-activated protein kinase 3 |
| E01 | MAPK8 | Mitogen-activated protein kinase 8 |
| E02 | NGF | Nerve growth factor (beta polypeptide) |
| E03 | NTRK1 | Neurotrophic tyrosine kinase, receptor, type 1 |
| E04 | OPRD1 | Opioid receptor, delta 1 |
| E05 | OPRK1 | Opioid receptor, kappa 1 |
| E06 | OPRM1 | Opioid receptor, mu 1 |
| E07 | P2RX3 | Purinergic receptor P2X, ligand-gated ion channel, 3 |
| E08 | P2RX4 | Purinergic receptor P2X, ligand-gated ion channel, 4 |
| E09 | P2RX7 | Purinergic receptor P2X, ligand-gated ion channel, 7 |
| E10 | P2RY1 | Purinergic receptor P2Y, G-protein coupled, 1 |
| E11 | PDYN | Prodynorphin |
| E12 | PENK | Proenkephalin |
| F01 | PLA2G1B | Phospholipase A2, group IB (pancreas) |
| F02 | PNOC | Prepronociceptin |
| F03 | PROK2 | Prokineticin 2 |
| F04 | PTGER1 | Prostaglandin E receptor 1 (subtype EP1), 42 kDa |
| F05 | PTGER3 | Prostaglandin E receptor 3 (subtype EP3) |
| F06 | PTGER4 | Prostaglandin E receptor 4 (subtype EP4) |
| F07 | PTGES | Prostaglandin E synthase |
| F08 | PTGES2 | Prostaglandin E synthase 2 |
| F09 | PTGES3 | Prostaglandin E synthase 3 (cytosolic) |
| F10 | PTGS1 | Prostaglandin-endoperoxide synthase 1 (prostaglandin G/H synthase and cyclooxygenase) |
| F11 | PTGS2 | Prostaglandin-endoperoxide synthase 2 (prostaglandin G/H synthase and cyclooxygenase) |
| F12 | SCN10A | Sodium channel, voltage-gated, type X, alpha subunit |
| G01 | SCN11A | Sodium channel, voltage-gated, type XI, alpha subunit |
| G02 | SCN3A | Sodium channel, voltage-gated, type III, alpha subunit |
| G03 | SCN9A | Sodium channel, voltage-gated, type IX, alpha subunit |
| G04 | SLC6A2 | Solute carrier family 6 (neurotransmitter transporter, noradrenalin), member 2 |
| G05 | TAC1 | Tachykinin, precursor 1 |
| G06 | TACR1 | Tachykinin receptor 1 |
| G07 | TLR2 | Toll-like receptor 2 |
| G08 | TLR4 | Toll-like receptor 4 |
| G09 | TNF | Tumor necrosis factor |
| G10 | TRPA1 | Transient receptor potential cation channel, subfamily A, member 1 |
| G11 | TRPV1 | Transient receptor potential cation channel, subfamily V, member 1 |
| G12 | TRPV3 | Transient receptor potential cation channel, subfamily V, member 3 |

Description of 13 inflammation genes discussed in the Pain Management experimental study.

| Array | Gene | |
|---|---|---|
| Code | Label | Description (role in inflammation) |
| A01 | ACE | ACE is a key enzyme of the brain rennin-angiotensin system for blood pressure regulation, water homeostasis and |
| | | secretion of hormones. ACE has also been shown to play a role in neutrophil immune response to bacterial |
| | | infection. Upregulation of ACE would suggest an inflammatory state requiring the recruitment of neutrophils. |
| A05 | BDKRB1 | Part of the kallikrein-kinin system (KKS), upregulation of BDKRB1 is strongly induced during inflammation |
| | | events. |
| B07 | CSF1 | A growth factor that selectively promotes the proliferation, survival, and differentiation of mononuclear phagocytic |
| | | cells. CSF-1 is upregulated by both IL-1β and TNFα resulting in inflammation |
| C01 | GCH1 | GCH1 protein functions as the rate-limiting enzyme of a metabolic pathway synthesizing tetrahydrobiopterin |
| | | (BH4). Pro-inflammatory factors including IL-1β, TNFα and IFNγ all upregulate the GHC1 gene causing an |
| | | increase of BH4. BH4 binds with high affinity to nitric oxide synthase (NOS)-2 to activate the enzyme and create |
| | | cytotoxic peroxynitrite radical (ONOO—) which is harmful to neurons as a part of the inflammatory response. |
| C02 | GDNF | GDNF protein in healthy tissues promotes the survival of many types of neurons. However, in a neuroinflammatory |
| | | state GDNF is upregulated, a detriment to neuronal activity and survival. |
| C11 | IL1A | IL-1α is expressed under homeostatic conditions but is upregulated many different inflammatory stimuli. After |
| | | injury or insult, IL-1α expression increases which signals IL-1R to induce downstream inflammatory responses. |
| | | This IL-1R cascade can activate downstream inflammatory factors, including COX-2, IL-6, and TNF, and further |
| | | upregulate IL-1α and IL-1β. |
| C12 | IL1B | In the central nervous system, microglia activated microglia release IL-1β after neural insults causing a feedback |
| | | cycle between astrocytes, microglia, and neurons to further release inflammatory mediators. Upregulation of IL-1β |
| | | in astrocytes would model an inflammatory and pain state. |
| D02 | IL6 | Functions as a mediator for notification of an emergent event. In the event of an injury, infection, inflammation or |
| | | disease, IL-6 levels increase significantly. This upregulation may be mediated in part by IL-1β, TNFα and IFNγ |
| | | when in an inflammatory state. |
| D04 | ITGB2 | Encodes the beta unit CD18 which forms heterodimers which function in cellular adhesion and cell surface |
| | | signalling, particularly in immune responses. In an inflammatory state, expression of ITGB2 would increase to |
| | | strongly recruit immune cells to affected areas. |
| F07 | PTGES | PTGES functionally works to synthesize the prostaglandin PGE2. |
| | | Proinflammatory cytokines such as IL-1β and TNFα activate transcription factor NF-κB which in turn regulates |
| | | PTGES. |
| | | Upon inflammatory insult, PTGES is upregulated to produce PGE2 which is known to induce the sensitization of |
| | | nociceptive neurons. |
| F11 | PTGS2 | PTGS2 (or COX-2) is functionally activated to produce the prostaglandin PGE2. Proinflammatory cytokines such |
| | | as IL-1β and TNFα activate transcription factor NF-κB which in turn regulates PTGS2. Upon inflammatory insult, |
| | | PTGS2 is upregulated to produce PGE2 which is known to induce the sensitization of nociceptive neurons. |
| G07 | TLR2 | Part of a family of pattern recognition receptors that recognize pathogen-associated molecular patterns. |
| | | TLR2 has been shown to be activated by TNFα via NF-κB signalling pathway which is upregulated due to |
| | | inflammatory insults or pathogen infiltration. |
| G09 | TNF | TNFα is an important mediator of inflammatory responses. TNFα is upregulated upon inflammatory insult, or via |
| | | activation other pro-inflammatory cytokines such as IL-1β and IFN-γ. Upregulation of TNFα in astrocytes also |
| | | triggers the production of IL-6. |

### Examples of Non-Limiting Aspects of the Disclosure

Aspects, including embodiments, of the present subject matter described herein may be beneficial alone or in combination, with one or more other aspects or embodiments. Without limiting the foregoing description, certain non-limiting aspects of the disclosure numbered 1-66 are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:
1. A method for treatment of an injury or disease in a patient using electromagnetic fields, the method comprising:
   determining an ideal personalized microenvironment stimulation target (PMST) to elicit a desired biological response at the microenvironment of the injury or disease; and
   generating a personalized treatment protocol (PTP) for the patient based on a prescribed electromagnetic field modality, the PTP configured to achieve the PMST required for the ideal personalized electromagnetic field (EMF) stimulation of the microenvironment of the injury or disease,
   wherein the PMST is calculated from patient-centric data and clinical meta data, and
   wherein the PTP is calculated to achieve the PMST given the properties of the EMF modality-centric data.
2. The method of claim 1, wherein the PMST is calculated by a Microenvironment Computational Engine (MiCE) configured with one or more physics-based computational algorithms for integrating and processing the patient-centric data and clinical meta data
3. The method of claim 2, wherein the MiCE utilizes organized indexed collections of said patient-centric data representing multidimensional parameters comprising: biology of the patient; biological challenge of the patient's injury or disease microenvironment targeted for treatment; and clinical metadata.
4. The method of claim 3, wherein biology of the patient comprises data relating to one or more of:
   overall patient characteristics and conditions that influence the microenvironment;
   how the patient characteristics and conditions influence the microenvironment;
   demographics comprising age, sex, height and weight; and
   comorbidities, smoking status, current medications, and existing medical conditions.
5. The method of claim 3 or 4, wherein biological challenge comprises data related to one or more of the unique detail of the patient's injury or disease, the microenvironment of the injury or disease, the severity of the injury or disease, the current state of recovery of the injury or disease, and the success or failure of prior treatments of the injury or disease.
6. The method of any one of claims 3 to 5, wherein the clinical data comprises any available data pertaining to the biology of similar patient and similar microenvironment.
7. The method of any one of claims 1 to 6, wherein the PTP is generated by a Macrotranslation Computational Engine (MaCE) configured with one or more physics-based computational algorithms for integrating and processing EMF-centric data.
8. The method of claim 7, wherein the EMF-centric data comprises organized indexed collections of data representing multidimensional parameters of the prescribed electromagnetic field modality to calculate the PTP required to achieve the PMST.
9. The method of claim 7 or 8, wherein the PTP defines an EMF that achieves the PMST required by the microenvironment of the injury or disease of the patient to influence mediators of inflammation and/or biological factors present at said microenvironment.
10. The method of claim 8 or 9, wherein data representing multidimensional parameters of the prescribed electromagnetic field modality comprises:
   transmission pathway data representing the signal pathway separating the microenvironment and EMF source that is influenced by material properties and physical dimensions of tissues and materials along the signal pathway; and
   deployment specifics data representing the modality of electromagnetic field generation and physical construction of EMF signal generator device as configured to the patient and the injury or disease.
11. The method of any one of claims 2 to 10, wherein the method further comprises an optimization extension comprising one or more computational engines that gather information from sensors at the microenvironment and external to the microenvironment to provide feedback to optimize the MiCE and the MaCE.
12. The method of claim 11, wherein said one or more optimization engines is a Feedback Computational Engine that acquires input data from EMF sensors at or near the microenvironment and configured to determine and correct for differences between the target EMF defined by the MaCE and actual measured EMF, as a result of any inaccuracies in the MaCE, and send any corrections as feedback to the parameters influencing the generation of the PTP.
13. The method of claim 11 or 12, wherein said one or more optimization engines is a Learn Computational Engine configured for optimization during active treatment via a self-contained feedback loop acquiring input data from follow-up and/or microenvironment sensor data compensate for inaccuracies in the MICE.
14. The method of claim 13, wherein the compensation for inaccuracies in the MICE comprises:
   (a) incrementally adjusting individual parameters of the PTP including an increase and decrease of frequency or intensity of stimulation;
   (b) monitoring effect(s) of any change(s) due to the adjusting in (a);
   (c) collecting sensor data and mapping a patient-specific response profile for each parameter; and
   (d) selecting an optimal combination of settings for the patient's recovery and output an optimized PTP.
15. The method of any one of claims 1 to 14, wherein newly emerging data comprising interim data and final treatment data are acquired, the interim data is fed back into the MiCE for improving the effectiveness of the PMST calculation and the final treatment data are integrated into the clinical metadata for calculating a PMST for a future patient.
16. The method of any one of claims 1 to 15, further comprising collecting progress data throughout the method, said progress data comprising clinical follow-up data and clinical sensor(s) data from clinical sensors located at the microenvironment.
17. The method of claim 16, wherein the progress data comprises one or more of radiographic data, functional assessment data, biomarker assay data, real-time biosensor(s) data, compliance metric(s) data, or direct patient input data.
18. The method of any one of claims 1 to 17, wherein said PTP influences biological processes at the microenvironment involved in for example: stabilizing, reversing and/or improving state of the injury or disease; improving/restoring function of the injury or tissue/organ affected by the disease; decrease spread/growth of the disease; stabilize the injury or disease; and manage/decrease pain associated with the injury or disease.
19. The method of any one of claims 1 to 18, wherein application of said PTP induces bioelectric effects at the cellular level within the microenvironment.
20. The method of any one of claims 1 to 19, wherein achieving said PMST more precisely targets biochemical and biophysical pathways of cells and associated structures in the microenvironment of the injury or disease encouraging cellular growth, tissue growth, repair, and maintenance.
21. The method of any one of claims 1 to 20, wherein achieving said PMST stimulates/modulates biochemical markers at the microenvironment including cytokines, growth factors, tumor markers, inflammatory markers, endocrine markers and metabolic markers.
22. The method of claim 21, wherein said stimulation/modulation of the biochemical markers enhances blood flow for accelerating repair of cells, organs and tissues, and modulating angiogenesis and neovascularization.
23. The method of any one of claims 1 to 22, wherein achieving said PMST:
   (a) stimulates/modulates physiologically relevant pathways of the microenvironment, including general transmembrane potential changes involved in stabilizing, reversing, healing of injury or disease; and/or
   (b) stimulate physiological induced changes including variations in cell membrane, enzymatic activity, cell apoptosis, nerve conduction, collagen synthesis, vasodilation, vasoconstriction, viscosity of body fluids/blood, pain signaling, production of endorphins, tissue metabolism, blood flow, inflammation, supply of oxygen & nutrients, tissue/muscle repair or healing, fibroblast activity, collagen fibril density, protein synthesis, and tissue regeneration.
24. The method of any one of claims 1 to 22, wherein the PTP defines an effective treatment duration to effect healing of the injury or disease.
25. The method of any one of claims 1 to 24, wherein the injury or disease involves one of more of cancer, cardiovascular disease, inflammatory disease, autoimmune disease, neurological disease, musculoskeletal pain management, wound repair, bone repair, osteoporosis, tissue repair, rehabilitation of traumatic injury, a sports injury and surgical rehabilitation.
26. The method of claim 25, wherein the injury is a bone fracture including, but not limited to accidental occurrences or deliberate surgical intervention, simple fracture (closed fractures); compound fracture; oblique fracture; transverse fracture; spiral fracture; comminuted fracture; liner fracture; greenstick fracture, partial fracture; impacted fracture; complete and incomplete fractures; compression fracture; avulsion fracture; stress fracture; hairline fracture; displaced fracture; non-displaced fracture; fatigue fracture; pathological fracture; and non-union.
27. The method of claim 25, wherein the injury is torn cartilage and the method is for accelerating healing of damaged or torn cartilage.
28. The method of claim 25, wherein the disease is a bone disease selected from osteoporosis, metabolic bone disease, bone cancer, and scoliosis.
29. The method of claim 25, wherein the disease is osteoarthritis, rheumatoid arthritis, spondyloarthritis, juvenile idiopathic arthritis, lupus, gout, and bursitis.
30. The method of claim 25, wherein the disease is cancer and said method is optionally in conjunction with surgery, radiation therapy and chemotherapy.
31. The method of claim 30, wherein said cancer is breast cancer, skin cancer, bone cancer, prostate cancer, liver cancer, lung cancer, brain tumor (glioma), head and neck cancers, colon cancer, osteosarcoma, small cell lung tumor, smooth muscle tumor, osteosarcoma and other sarcomas.
32. The method of claim 31, wherein said method reduces uncontrolled cell division and, after cancer tumor eradication, helps to regenerate tissue/organs to health and function which includes stem cell homing, controlled proliferation, differentiation and blood vessel sprouting, growth and maturation of protein expression.
33. The method of any one of claims 1 to 25, wherein said method is for treatment of pain.
34. The method of any one of claims 1 to 33, further comprising transmitting PTP and optionally optimization engine programmes to an EMF generator device.
35. An EMF signal generator device for delivering the PTP that achieves the PMST in the patient injury or disease microenvironment in accordance with any one of claims 1 to 34.
36. The EMF signal generator device of claim 35, comprising:
   a software-based controller;
   an EMF signal generator capable of running time-based sequences defined by the software-based controller;
   at least one EMF source in operable communication with the EMF signal generator, wherein the EMF source achieves the PMST in the microenvironment of the injury or disease.
37. A system comprising the EMF signal generator device of claim 35 or 36.
38. A device for providing an electromagnetic field (EMF) personalized to an injury or disease microenvironment in a patient, the device comprising:
   one or more software-based controller(s) comprising a MiCE, MaCE and optionally optimization engines;
   an EMF signal generator capable of running sequences defined by the software-based controller(s); and
   at least one EMF source in operable communication with the EMF signal generator, wherein the at least one EMF source is configured to achieve the PMST in the microenvironment of the injury or disease.
39. The device of claim 38, wherein the PMST is computed by a Microenvironment Computational Engine (MiCE).
40. The device of claim 39, wherein MiCE comprises:
   one or more physics-based computational algorithms;
   organized indexed collections of data representing multidimensional parameters of:
      a patient,
      the biology of the patient's microenvironment targeted for treatment,
      and clinical metadata pertaining to the biology of a similar target microenvironment;
   wherein the MiCE calculates the patient-specific and theoretically ideal personalized microenvironment stimulation target required to elicit the required biological response in the microenvironment of the injury or disease.
41. The device of any one of claims 38 to 40, wherein the personalized treatment protocol is computed by a Macrotranslation Computational Engine (MaCE).
42. The device of claim 41, wherein the Macrotranslation Computational Engine (MaCE) comprises:
   one or more physics-based computational algorithms;
   organized indexed collections of data representing multidimensional parameters of an electromagnetic field modality prescribed for the patient;
   and the properties of the transmission pathway separating the electromagnetic field source from the location of the personalized microenvironment stimulation target.
   wherein, the MaCE calculates the precise personalized electromagnetic field output from an EMF source to provide an exact stimulus required at a target microenvironment of the patient to stimulate healing of the patient's injury or disease.
43. The device of any one of the claim 41 or 42, wherein the MiCE computes the ideal stimulation at the microenvironment of a patient injury or disease and the MaCE computes the ideal operation of an EMF source to generate the ideal stimulation at the microenvironment.
44. The device of any one of claims 38 to 43, wherein the personalized treatment protocol comprises a PEMF signal.
45. The device of any one of claims 38 to 44, wherein the EMF source is configured for inductive coupling.
46. The device of any one of claims 38 to 44, wherein the EMF source is configured for capacitive coupling using electrodes for electrochemical contact with a surface of the injury or disease.
47. The device of any one of claims 38 to 45, wherein the EMF source comprises one or more wire coils with a plurality of coplanar loops.
48. The device of claim 47, wherein the signal applicator comprises one or more shaped coil.
49. The device of any one of claims 38 to 48, wherein the device is configured as a wearable device.
50. The device of claim 49, wherein the wearable device is selected from an anatomical wrap, anatomical support, apparel, chest support, hat, cap, helmet, foot ware, dressing, bandage, compression bandage and compression dressing.
51. The device of any one of claims 38 to 50, wherein the device and/or components thereof are configured to be re-useable.
52. The device of any one of claims 38 to 51, wherein the device further comprises a replaceable or rechargeable power source.
53. The device of any one of claims 38 to 52, wherein the device and/or components thereof are configured to be disposable, recyclable and/or replaceable.
54. The device of any one of claims 38 to 53, wherein the device and/or components thereof are configured to be incorporated into device in routine close proximity to a patient, including a mattress, mattress pad, linen, furniture, exercise equipment, automobile or support device.
55. A wearable electromagnetic field therapy system, the system comprising:
   an EMF signal generator;
   a microcontroller;
   one or more wire coil EMF sources operationally coupled to the EMF signal generator; and
   a material configured to secure the EMF sources adjacent an area of injury or disease;
   wherein the microcontroller is configured to generate a personalized treatment protocol (PTP) that achieves an ideal personalized microenvironment stimulation target (PMST) to elicit the required biological response in the microenvironment of the injury or disease.
56. A computer implemented EMF treatment system comprising:
   a signal generator comprising a memory or chip storing:
   a microenvironment computational engine for calculating a personalized microenvironment stimulation target (PMST) for a patient prescribed bioelectromagnetic therapy for treatment of an injury or disease; and
   a macrotranslation computational engine for generation of a personalized treatment protocol (PTP); and
   one or more EMF sources coupled to the signal generator for delivery of the PTP to achieve the PMST.
57. A bioelectromagnetic therapy system comprising:
   a microenvironment computational engine configured to calculate the theoretically ideal personalized microenvironment stimulation target (PMST) specific to a treatment of a patient;
   a macrotranslation computational engine configured to generate a personalized treatment protocol (PTP) to achieve the theoretically ideal personalized microenvironment stimulation target; and
   a signal generator device configured to implement the personalized treatment protocol.
58. A software product comprising machine-executable code for execution of a Microenvironment Computational Engine (MiCE), comprising:
   a series of physics-based computational algorithms;
   organized indexed collections of data representing multidimensional parameters of:
      a patient,
      the biology of the patient's microenvironment targeted for treatment, and
      clinical metadata pertaining to the biology of a similar target microenvironment,
   wherein, MiCE calculates a patient-specific and theoretically ideal Personalized Microenvironment Stimulation Target (PMST) to elicit the required biological response in the microenvironment of the injury or disease.
59. A software product comprising machine-executable code for execution of a Macrotranslation Computational Engine (MaCE), comprising:
   a series of physics-based computational algorithms; transmission pathway data representing the signal pathway separating the microenvironment and EMF source that is influenced by material properties and physical dimensions of tissues and materials along the signal pathway; and
   deployment specifics data representing the modality of electromagnetic field generation and physical construction of EMF signal generator device as configured to the patient and the injury or disease;
   wherein, the MaCE calculates the precise personalized electromagnetic field output from an EMF source to provide an exact stimulus required at a target microenvironment of the patient to stimulate healing of the patient's injury or disease.
60. A therapeutic garment configured for applying personalized electromagnetic field (EMF) stimulation for treatment of an injury or disease in a patient, the therapeutic garment comprising:
   a plurality of wire coil EMF sources integrated into the garment; and
   an EMF signal generator integrated into garment and connected to the plurality of wire coil EMF sources, wherein the EMF signal generator is configured to deliver a personalized treatment protocol (PTP) to achieve the personalized microenvironment stimulation target (PMST).
61. A wearable device, comprising:
   a mount for mounting the wearable device to a surface of a patient's body proximate to a microenvironment of an injury or disease in the patient;
   an EMF signal generator operationally connected to one or more EMF sources;
   a communication interface;
   a processor;
   a non-transitory computer-readable medium; and
   computational engines stored in the non-transitory computer-readable medium each executable by the processor to cause the wearable device to perform functions comprising: calculating a personalized microenvironment stimulation target (PMST) signal, and setting a personalized treatment protocol (PTP) for the patient based on the device modality, the PTP configured to achieve the PMST at the microenvironment of the injury or disease, and
   optionally transmitting data representative of the microenvironment of the injury or disease via the communication interface.
62. The wearable device of claim 61, wherein said wearable device is selected from the group consisting of an anatomical wrap, anatomical support, apparel, chest support, hat, cap, helmet, foot ware fashion accessory, dressing, bandage, compression bandage and compression dressing.
63. A method of treating an injury or disease of a patient's body, the method comprising:
   placing an electromagnetic treatment device substantially adjacent a region of the patient's body to be treated;
   activating the electromagnetic treatment device to generate a personalized microenvironment stimulation target (PMST) applied by a capacitively coupled EMF source, the PMST calculated to provide the ideal electromagnetic stimulation required for healing of the injury or disease of the patient.
64. A system, comprising:
   a bioelectromagnetic therapy device comprising:
   an EMF signal generator configured to deliver a personalized treatment protocol (PTP) to achieve the personalized microenvironment stimulation target (PMST) ideal for stimulating a microenvironment of an injury or disease in a patient;
   at least one sensor;
   a processor operationally coupled to the EMF signal generator and the at least one sensor; and
   a memory communicatively coupled to the processor and comprising instructions configured to be executed by the processor, wherein the processor is configured to receive the instructions from the memory and to execute the instructions to perform operations comprising calculation of the PMST and determination of the PTP for the patient;
   delivering the PTP using at least one EMF source operationally coupled to the EMF signal generator to achieve the PMST in the microenvironment.
65. A system for providing a personalized electromagnetic signal targeted to a microenvironment of a patient, comprising:
   a microenvironment computational engine incorporating at least one of artificial intelligence, machine learning, computational and mathematical analysis; and
   a database of stored patient-centric information related to:
      physiological and/or biochemical data characterizing a biological challenge of the microenvironment,
      a patient profile comprising at least one of measured patient disease data and user data related to disease management, and
      clinical metadata,
   wherein the microenvironment computational engine comprises protocols using the at least one of artificial intelligence, machine learning, computational and mathematical analysis to integrate and process the database of stored patient-centric information and compute the personalized electromagnetic signal uniquely targeted to the microenvironment.
66. The system of claim 65, wherein the system further comprises:
   a macrotranslation computational engine incorporating at least one of artificial intelligence, machine learning, computational and mathematical analysis; and
   a database of stored EMF-modality centric information related to macrotranslation factors concerning deployment of the personalized electromagnetic signal,
   wherein said macrotranslation computational engine comprises protocols using the at least one of artificial intelligence, machine learning, computational and mathematical analysis to integrate the computed personalized electromagnetic signal and database of stored EMF-modality centric information to output a personalized treatment protocol′ (PTP) for the patient

The descriptions of the various embodiments and/or examples of the present invention have been presented for purposes of illustration but are not intended to be exhaustive or limited to the embodiments and/or examples disclosed. Many modifications and variations will be apparent to those of ordinary skill in the art without departing from the scope and spirit of the described embodiments. The terminology used herein was chosen to best explain the principles of the embodiments, the practical application, or to enable further understanding of the embodiments disclosed herein.

1. A method for treatment of an injury or disease in a patient using electromagnetic fields, the method comprising:
determining an ideal personalized microenvironment stimulation target (PMST) to elicit a desired biological response at the microenvironment of the injury or disease; and
generating a personalized treatment protocol (PTP) for the patient based on a prescribed electromagnetic field modality, the PTP configured to achieve the PMST required for the ideal personalized electromagnetic field (EMF) stimulation of the microenvironment of the injury or disease,
wherein the PMST is calculated from patient-centric data and clinical meta data, and
wherein the PTP is calculated to achieve the PMST given the properties of the EMF modality-centric data.

2-66. (canceled)
